(19) United States
(12) Patent Application Publication
(10) Pub. No.: US 2024/0002467 A1
(43) Pub. Date: Jan. 4, 2024

(54) COMPOSITIONS AND METHODS OF INHIBITING THE BINDING OF PLASMA IGG AUTOANTIBODIES TO SEROTONIN 2A RECEPTOR

(71) Applicant: UNITED STATES GOVERNMENT AS REPRESENTED BY THE DEPARTMENT OF VETERANS AFFAIRS, Washington, DC (US)

(72) Inventor: Mark B. Zimering, Lyons, NJ (US)

(21) Appl. No.: 18/038,588

(22) PCT Filed: Nov. 22, 2021
(86) PCT No.: PCT/US2021/060306

§ 371 Date: May 24, 2023

Related U.S. Application Data
(60) Provisional application No. 63/117,491, filed on Nov. 24, 2020.
(60) Provisional application No. 63/246,691, filed on Sep. 21, 2021.

(51) Int. Cl. C07K 14/705; A61K 47/54; G01N 33/564; A61P 3/06; A61P 3/04; A61P 9/12; A61K 31/506; A61K 31/445

(52) U.S. Cl. CPC C07K 14/70571; A61K 47/542; G01N 33/564; A61P 3/06; A61P 3/04; A61P 9/12; A61K 31/506; A61K 31/445; G01N 2333/70571; G01N 2333/91188

(57) ABSTRACT

Disclosed herein, are decoy peptides or polypeptides capable of inhibiting binding of 5-HT2A autoantibodies to a second extracellular loop region of the 5-HT2A receptor, and a pharmaceutical composition containing the decoy peptides or polypeptides and methods of use.

### CROSS REFERENCE TO RELATED APPLICATIONS

This application claims the benefit of U.S. Provisional Application Nos. 63/117,491, filed Nov. 24, 2020; and 63/246,691, filed Sep. 21, 2021. The content of these earlier filed applications is hereby incorporated by reference herein in their entirety.

### STATEMENT REGARDING FEDERALLY SPONSORED RESEARCH

This invention was made with government support under Grant Number TTP-001-awarded by the United States Department of Veterans Affairs. The government has certain rights in this invention.

### INCORPORATION OF THE SEQUENCE LISTING

The present application contains a sequence listing that is submitted via EFS-Web concurrent with the filing of this application, containing the file name “37759_0355P1_SL.txt” which is 28,672 bytes in size, created on Nov. 22, 2021, and is herein incorporated by reference in its entirety.

### BACKGROUND

Obesity and obese type 2 diabetes mellitus are associated with moderate hypertriglyceridemia, the latter is an independent risk factor for premature atherosclerosis, atherosclerotic heart disease stroke occurrence and fatty liver disease. Yet few if any current FDA-approved medications for the treatment of moderate hypertriglyceridemia achieves a plasma triglyceride-lowering effect greater than 18-20%. On the other hand, sustained significant weight loss can result in substantially greater triglyceride-lowering effect. In most patients, however, lifestyle modification needed to realize sustained significant weight loss is difficult to attain. Thus, new pharmacologic compositions which promote significant weight loss resulting in substantial plasma triglyceride-lowering effect that is capable of lowering the risks of cardiovascular disease occurrence and/or liver dysfunction due to fatty infiltration is needed.

### SUMMARY

Disclosed herein are methods of lowering plasma triglycerides in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of lowering alanine transaminase (ALT) in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating a subject having or at risk of developing hypertriglyceridemia, wherein the subject has type 2 diabetes, the methods comprising: administering to the subject a therapeutically effective amount of a 5-HT2A receptor antagonist, wherein the subject has been identified to have or to be at risk of developing hypertriglyceridemia by identifying elevated levels of triglycerides in a sample obtained from the subject.

Disclosed herein are methods of treating a subject having or at risk of developing hypertriglyceridemia, wherein the subject is obese, the methods comprising: administering to the subject a therapeutically effective amount of a 5-HT2A receptor antagonist, wherein the subject has been identified to have or be at risk of developing hypertriglyceridemia by identifying elevated levels of triglycerides in a sample obtained from the subject.

Disclosed herein are methods of treating hypertriglyceridemia in a subject, the methods comprising: a) detecting elevated levels of triglycerides in a sample obtained from the subject; b) detecting the presence of autoantibodies to 5-HT2A in the sample obtained from the subject; c) administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating obesity in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating diabetic dyslipidemia in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating premature atherosclerosis in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating atherosclerotic heart disease in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are method of treating a subject at risk for having a stroke or preventing or ameliorating a symptom of stroke in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are method of treating hypertension in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of treating fatty liver disease in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods reducing the risk of developing liver cirrhosis or liver failure in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of preventing cardiac steatosis in a subject, the methods comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

Disclosed herein are methods of reducing or preventing cardiac hypertrophy in a subject, the method comprising: administering to the subject a therapeutically effective amount of a decoy peptide, wherein the decoy peptide comprises or consists of the sequence QDDSKVFKEGSCLLADDN (SEQ ID NO: 1), or a fragment thereof.

### BRIEF DESCRIPTION OF THE DRAWINGS

FIGS. 1A-B show that chronic decoy peptide treatment is associated with changes in ALT levels but not AST levels. FIG. 1A shows that chronic treatment with decoy peptide 2 is not associated with significant increase in AST in 16-week old ZDF rats. FIG. 1B shows that chronic treatment with decoy peptide 2 is associated with lower hepatic enzyme (ALT) in 16 week old ZDF rats.

FIG. 2 shows the triglyceride-lowering effects of the decoy peptide-2 compared to saline-treated 16 week old Zucker diabetic fatty rats.

FIGS. 3A-C show body weight changes in ZDF rats treated with chronic decoy peptide 2 treatment or saline. FIG. 3A shows changes in body weight in ZDF rats during saline treatment. Each line represents one ZDF rat. FIG. 3B shows the body weight trajectory in untreated ZDF rats. Each point is the average and standard deviation of two rats. FIG. 3C shows changes in body weight in ZDF rats during chronic treatment with decoy peptide 2. Each line represents one ZDF rat.

FIG. 4 shows the mean change in body weight in ZDF rats treated with saline or decoy peptide 2 between 10 and 16 weeks.

FIGS. 5A-B show weight change and food consumption in ZDF rats during matching 1 week periods of OFF vs ON decoy peptide 2. FIG. 5A shows the matching 1-week periods of OFF and ON decoy peptide 2 treatment, and the effects on body weight. FIG. 5B shows that decoy peptide 2 treatment lowers food consumption.

FIG. 6 shows plasma triglyceride levels at 23 weeks of age in decoy peptide 2- vs saline-treated ZDF rats.

FIG. 7 shows the change in plasma triglyceride levels over 5-6 week time periods in ZDF rats. *saline vs decoy peptide 2-treated from week 16-22; \hat{ } untreated from week 14-20.

FIG. 8 shows that heart weight was significantly increased (mean 1.55 g vs. 1.2. g) in ZDF vs lean rats (from the study by Zhou et al, Proc Natl Acad Sci USA, Feb. 15, 2000:97, pp. 1784-1789) in saline vs. decoy peptide 2 treated rats as well as untreated rats.

FIG. 9 shows the systolic blood pressure-lowering effect of PEGylated decoy peptide 2 vs scrambled decoy peptide 2.

FIG. 10 shows the diastolic blood pressure-lowering effect of the pegylated decoy peptide 2 vs scrambled pegylated decoy peptide.

FIG. 11 shows that pegylated decoy peptide 2 (0.75 mg/kg, IP) causes long-lasting systolic blood pressure-lowering in ZDF rats compared to 2 mg/kg IP of a scrambled peptide sequence.

FIG. 12 shows that pegylated decoy peptide 2 (0.75 mg/kg IP) causes long-lasting diastolic blood pressure-lowering in ZDF rats compared to 2 mg/kg IP of a scrambled peptide sequence.

FIGS. 13A-B show that a single intraperitoneal dose of decoy peptide 2 (2 mg/kg) acutely lowers systolic (FIG. 13A) and diastolic blood pressure (FIG. 13B) in the Zucker diabetic fatty rat. Each point represents the mean+/SEM values in two-three, 25-week-old male ZDF rats.

FIGS. 14A-B show that decoy peptide 2 (2 mg/kg), single intraperitoneal injection, causes long-lasting systolic (FIG. 14A) and diastolic blood pressure-lowering (FIG. 14B) effects in 25-week-old male ZDF rats. Each point represents the mean+/SEM values.

FIGS. 15A-C show that pegylated decoy peptide 2 (1 mg/kg), single intraperitoneal injection, caused acute systolic blood pressure lowering in 8.5-week-old male ZDF rats (FIG. 15A), an effect that was not observed after (2 mg/kg) injection of scrambled decoy peptide 2 (FIG. 15B or sterile saline (vehicle) (FIG. 15C). Each point represents the mean+/SEM values.

FIGS. 16A-B show that single intraperitoneal dose of pegylated decoy peptide 2 (1 mg/kg) causes significantly greater systolic (FIG. 16A) and diastolic blood pressure-lowering (FIG. 16B) compared to scrambled decoy peptide peptide (2 mg/kg).

FIGS. 17A-B show that single intraperiotoneal dose of pegylated decoy peptide 2 (0.75 mg/kg) causes long-lasting significant systolic (FIG. 17A) and diastolic (FIG. 17B) blood pressure-lowering compared to scrambled decoy peptide 2 (2 mg/kg)/in ZDF rats.

FIG. 18 shows that chronic administration of alternate daily intraperitoneal decoy peptide 2 (2 mg/kg) was associated with significantly lower heart-to-body weight ratio than in baseline matched ZDF rats treated with IP scrambled decoy peptide 2 (2 mg/kg). Results are mean+/−SEM.

FIGS. 19A-B show the long-lasting blood pressure-lowering effects in two male ZDF rats after a single IP administration of 2.5 mg/kg of myristolated decoy peptide 2 (SEQ ID NO: 9). FIG. 19A shows persistant blood pressure lowering for 8 days after a single IP dose (2.5 mg/kg) of myristolated decoy peptide 2 (SEQ ID NO: 9) in two thirteen week old male ZDF rats. Results are mean+/SEM of 3-5 blood pressure readings at each time point. *P<significantly difference compared to day 0 (baseline) systolic or diastolic blood pressure. FIG. 19B shows normalization of baseline blood pressure level nine days after a single IP dose (2.5 mg/kg) of myristolated decoy peptide 2 (SEQ ID NO: 9) in two thirteen week old male ZDF rats. Results are mean+/SEM of 3-5 blood pressure readings at each time point. NS—no statistically significant difference between mean values at day 0 vs day 9 for systolic or diastolic blood pressure.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

The terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a compound” includes mixtures of compounds, reference to “a pharmaceutical carrier” includes mixtures of two or more such carriers, and the like.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment, such as, for example, prior to the administering step.

As used herein, the term “comprising” can include the aspects “consisting of” and “consisting essentially of.”

As used herein, the term “decoy peptide” or “decoy polypeptide” refers to a peptide or polypeptide designed to contain a partial peptide sequence in the second extracellular loop region of the 5-HT2A receptor, and the decoy peptide or polypeptide can block the action of receptor autoantibodies by binding to the 5-HT2A receptor autoantibodies. In some aspects, the 5-HT2A receptor autoantibodies can be IgG autoantibodies. For example, in some aspects, the decoy peptide or polypeptide can compete with the 5-HT2A receptor present, for instance, on cell surfaces of neurons and vascular cells, for binding to the soluble receptor autoantibodies or 5-HT2A receptor IgG autoantibodies.

The term “competitive inhibition” as used herein with reference to a decoy peptide or polypeptide can refer to an inhibition of the binding of 5-HT2A receptor autoantibodies to the receptor by binding to the 5-HT2A receptor autoantibodies. In some aspects, the decoy peptides or polypeptides disclosed herein can bind to the 5-HT2A receptor autoantibodies and as such that it competes with the second extracellular loop region of the 5-HT2A receptor. The 5-HT2A receptor is the targeting region for the 5-HT2A receptor autoantibodies present in the circulation.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; and Y, tyrosine.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the D- or L-configuration. If non-naturally occurring side chains are used, non-amino acid substituents may be used, for example to prevent or retard in vivo degradation.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

As used herein, “scrambled” “scrambled version”, or “scrambled peptide” is meant to mean that the composition of the amino acid sequence is the same as the unscrambled peptide, however, the sequence of the amino acids is altered thus rendering the peptide unable to inhibit autoantibodies from binding to the second extracellular loop region of the 5-hydroxytryptamine 2A (5-HT2A) receptor and activating the 5-HT2A receptor. For example, if one peptide has the amino acid sequence ABCDE, the scrambled version of the peptide could have the amino acid sequence DEABC.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Treatment” and “treating” refer to administration or application of a therapeutic agent (e.g., a decoy peptide or polypeptide described herein) to a subject or performance of a procedure or modality on a subject for the purpose of obtaining a therapeutic benefit of a disease or health-related condition. For example, a treatment may include administration of a pharmaceutically effective amount of a decoy peptide or polypeptide that inhibits the binding of 5-HT2A autoantibodies to a second extracellular loop region of the 5-HT2A receptor.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be neurologic disease or disorder or microvascular disease or disorder or a neurodegenerative disease or disorder. In some aspects, the subject has type 2 diabetes, a neurologic disease or disorder, a microvascular disease or disorder, hypertension, schizophrenia, retinitis pigmentosa, mild cognitive dysfunction, primary open angle glaucoma, diabetic dyslipidemia, fatty liver disease, type 2 hyperlipidemia, hypertriglyceridemia, microvascular angiopathy, diabetic kidney disease, Parkinson's disease, dementia, major depressive disorder, obesity, refractory hypertension, essential hypertension or has had a stroke or a traumatic brain injury, is overweight, or a combination thereof.

The term “fragment” can refer to a portion (e.g., at least 4, 5, 6, 7, 8, 9, 10, 11, 12, 13, 14, 15, 16, 17, etc. amino acids) of a peptide that is substantially identical to a reference peptide and retains the biological activity of the reference. In some aspects, the fragment or portion retains at least 50%, 75%, 80%, 85%, 90%, 95% or 99% of the biological activity of the reference peptide described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “prevent” or “preventing” refers to preventing in whole or in part, or ameliorating or controlling.

As used herein, the terms “PEG”, “polyethylene glycol”, or “poly(ethylene glycol)” as used herein refers to any water soluble poly(ethylene oxide), and includes molecules comprising the structure —(CH₂CH₂O)ₙ— where n is an integer from 2 to about 800. A commonly used PEG is end-capped PEG, wherein one end of the PEG is capped with a relatively inactive group such as an alkoxy while the other end is a hydroxyl group that may be further modified. An often-used capping group is methoxy and the corresponding end-capped PEG is often denoted mPEG. The notion PEG is often used instead of mPEG. Specific PEG forms of the invention are branched, linear, forked PEGs, and the like and the PEG groups are typically polydisperse, possessing a low polydispersity index of less than about 1.05. The PEG moieties of the invention will, for a given molecular weight, typically consist of a range of ethylene glycol (or ethyleneoxide) monomers. For example, a PEG moiety of molecular weight 2000 Da will typically consist of 43±10 monomers, the average being around 43 monomers. The term “PEGylated” refers to the covalent attachment of PEG to another molecule, such as any of the decoy peptides disclosed herein.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain; the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3); (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

The term “ω-amino-fatty acid” refers to fatty acids which feature an amino group at the distal carbon of the hydrocarbon chain thereof. The ω-amino-fatty acid moieties that are used in the context of the present invention can be saturated or unsaturated hydrocarbon chains. These moieties have a carboxylic group at one end of the hydrocarbon chain and an amine group at the other. The hydrocarbon chain connecting the carboxylic and amine groups in such an ω-amino-fatty acid moiety typically has from 3 to 32 carbon atoms.

Exemplary ω-amino-fatty acids include, without limitation, 4-amino-butyric acid, 6-amino-caproic acid, 8-amino-caprylic acid, 10-amino-capric acid (10-amino-decanoic acid), 12-amino-lauric acid (12-amino-dodecanoic acid), 14-amino-myristic acid (14-amino-tetradecanoic acid), 14-amino-myristoleic acid, 16-amino-palmitic acid (16-amino-hexadecanoic acid), 18-amino-stearic acid, 18-amino-oleic acid, 16-amino-palmitoleic acid, 18-amino-linoleic acid, 18-amino-linolenic acid and 20-amino-arachidonic acid.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Compositions and Methods

Diabetes is associated with a substantially increased risk of certain neurovascular and neurodegenerative complications, e.g., stroke, dementia, Parkinson's disease, major depressive disorder (Hu G, et al, Diabetes Care 2007; 30(4): 842-847), through complex and as yet poorly-defined mechanisms. Activation of 5-HT2A receptor IgG autoantibodies in plasma or serum from older adult diabetes suffering with major depressive disorder (Zimering M B, J Endocrinol Diabetes. 2018; 5(2)), Parkinson's disease or dementia (Zimering M B, J Endocrinol Diabetes. 2017; 4(4)), was identified. Acute neurite retraction and accelerated mouse neuroblastoma N2a cell death induced by the autoantibodies in cell culture was partially or completely prevented by co-incubation (of IgG autoantibodies) with selective antagonists of the 5-HT2A receptor (Zimering M B, J Endocrinol Diabetes. 2018; and Zimering M B, J Endocrinol Diabetes. 2017; 4(4)).

Zucker fatty rats have a mutated leptin receptor (Phillips et al., 1996) leading to hyperphagia (Kowalski et al., 1998) with obesity apparent from around 4 weeks of age (Wang et al., 2014). They are hyperinsulinemic (Trimble et al., 1986) and have poor glucose tolerance (Triscari et al., 1979) although are not overtly diabetic. The Zucker diabetic fatty (ZDF) rat is a substrain of the Zucker fatty rat, which was derived from hyperglycemic Zucker fatty rats to gain a model with diabetic features (Peterson et al., 1990). It is severely insulin resistant and males become overtly diabetic at 8-10 weeks, which is due to an inability of beta cells to compensate for insulin resistance, which is associated with changes in islet morphology (Tokuyama et al., 1995). Females do not tend to develop overt diabetes but diabetes can be induced by feeding a high-fat diet (Corsetti et al., 2000). As disclosed herein, the Zucker rat strain can be used to study the bioactivity of the 5-HT2AR autoantibodies. The 5-HT2A receptor is present in both Zucker diabetic fatty rats and in lean Zucker rats at early ages, however, the 5-HT2AR autoantibodies persists into mature adulthood (e.g., 25 weeks of age). The heterozygous lean (fa/+) Zucker rat has twice as high level of 5-HT2AR autoantibody as in ZDF rat. The presence of the 5-HT2AR autoantibody at some stage of development in the three Zucker subtypes: (fatty, diabetic), (lean heterozygous), and (lean lacking a known leptin receptor mutation) suggests 5-HT2AR autoantibody is a Zucker strain ‘trait.’ The Zucker lean rat described herein and used in the Examples described herein lack a known leptin receptor mutation. As the neurotoxic bioactivity of the 5-HT2AR autoantibody in ZDF rats have properties that resemble those of the agonist 5-HT2AR-targeting autoantibody in humans, the ZDF rat model can be used to study the effects of the compositions (e.g. decoy peptides) disclosed herein.

Morbid obesity is associated with the deposition of fat in visceral organs. In humans, when this occurs in the liver it causes non-alcoholic fatty liver disease (NAFLD) which can progress to non-alcoholic steatosis (NASH) leading in some patients to hepatic dysfunction and cirrhosis. In studies of human obesity, elevation in the hepatic enzyme aspartate aminotransferase (ALT) was correlated with visceral adipose tissue and with the inflammatory marker, high-sensitivity c-reactive protein [Verijjken et al., 2010]. The Zucker diabetic fatty rat is a well-studied model of obese type 2 diabetes mellitus. In the Zucker diabetic fatty rat, morbid obesity and prolonged severe hyperglycemia are associated with fatty infiltration into hepatocytes leading to ballooning degeneration and eventually hepatic deficiency at around 7-8 months of life [Sui et al., 2019]. White adipose tissue is a source of circulating non-esterified, free fatty acids which together with glucose are taken up in the liver and stored as triglyceride. Under conditions of relative insulin deficiency as occurs in poorly-controlled diabetes, hepatic triglyceride is packaged into very low-density lipoprotein particles which are secreted from the liver into the bloodstream. Normal desirable level of circulating plasma triglyceride in humans ranges between 50-150 mg/dL. Significantly higher level is associated with increased risk of coronary and cardiovascular disease occurrence in humans. The Zucker diabetic fatty (ZDF) rat manifests extreme hypertriglyceridemia due to morbid obesity, with circulating plasma triglyceride levels in excess of 500 mg/dL. In the ZDF rat, excess triglyceride is deposited in heart tissue causing ‘myocardial steatosis’ which was associated with reduced cardiac contractility [Zhou et al., 2000]. Excess circulating triglyceride stored in the liver causes hepatic steatosis which can lead to hepatic dysfunction and cirrhosis in humans. For example, in a published study of Zucker diabetic fatty rats who were treated for 6 months either with the glucose-lowering medication metformin or vehicle, the serum ALT was more than 3-times higher than the upper limit of normal (˜142 IU/L; normal 10-45 IU/L) in both groups of rats [Sui et al., 2019], suggesting that improved glucose level alone is not sufficient to prevent hepatic steatosis which is a marker for liver dysfunction.

The second extracellular loop region of several different G-protein coupled receptors lies adjacent to the receptors' orthosteric binding pocket (Wacker D, et al. Cell. 2017; 168(3):377-389). For example, the second extracellular loop region of the 5-HT2A receptor is located near the orthosteric binding pocket. Autoantibody binding in this region of the second extracellular loop may induce a conformational change causing receptor activation. In subsets of human dilated cardiomyopathy (Holthoff H P, et al. Circ Res. 2012; 111(6):675-84) or in eclampsia (Dechend R, et al., Hypertension. 2005; 45(4):742-6), spontaneously-occurring autoantibodies which targeted the second extracellular loop region of the beta-1-adrenergic or the angiotensin 2, type 1 receptor, respectively, caused G-protein coupled receptor activation. Described herein are the results of a study that tested whether IgG autoantibodies in older adult diabetics having angiopathic and/or neurodegenerative complications causes 5-HT2A receptor activation via binding to the second extracellular loop region of the 5-HT2A receptor.

The 5-HT2A receptor is highly expressed in specific brain regions underlying cognition, perception and mood regulation (Xu T, et al., Brain Res Bull. 2000; 51(6):499-505). It is also expressed on vascular smooth muscle cells where it plays a role in the regulation of arterial vascular tone (Alsip N, L, et al., J Vasc Res 1991; 28:537-541). Autoantibodies capable of targeting the 5-HT2A receptor in the central nervous system and the peripheral vasculature could serve as a biomarker for many disease complications associated with refractory hypertension (e.g. stroke, chronic kidney disease) and/or neurodegeneration (dementia, Parkinson's disease, macular degeneration, retinal degeneration).

Compositions

Disclosed herein are compositions, including pharmaceutical compositions comprising one or more of the decoy peptides or polypeptides described herein. As described herein, the decoy peptides or polypeptides are capable of inhibiting autoantibodies from binding to the second extracellular loop region of the 5-hydroxytryptamine 2A (5-HT2A) receptor and activating the 5-HT2A receptor.

Disclosed herein are decoy peptides or polypeptides that can comprise or consist of the amino acid sequence of QDDSKVFKEGSCLLADDN (SEQ ID NO: 1), or a fragment thereof. In some aspects, the decoy peptide can comprise a fragment of QDDSKVFKEGSCLLADDN (SEQ ID NO: 1). In some aspects, the fragment of QDDSKVFKEGSCLLADDN (SEQ ID NO: 1) can be SCLLADDN (SEQ ID NO: 2), QDDSKVF (SEQ ID NO: 3), or VFKEGSC (SEQ ID NO: 4). In some aspects, the fragment of SEQ ID NO: 1 can be between 4 and 9 amino acids in length. Disclosed herein are decoy peptides or polypeptides that can comprise or consist of the amino acid sequence of QDDSKVFKEGSCLLADDN (SEQ ID NO: 1), or a variant thereof. In some aspects, the decoy peptide or polypeptide comprises or consists of the amino acid sequence of SCLLADDN (SEQ ID NO: 2), QDDSKVF (SEQ ID NO: 3), or VFKEGSC (SEQ ID NO: 4). In some aspects, the decoy peptide or polypeptide comprises or consists of the amino acid sequence of SCLLADDN (SEQ ID NO: 2), QDDSKVF (SEQ ID NO: 3), VFKEGSC (SEQ ID NO: 4) or variants thereof. In some aspects, the decoy peptide or polypeptide can inhibit the binding of 5-HT2A receptor autoantibodies to a second extracellular loop region of the 5-HT2A receptor. In some aspects, the decoy peptide or polypeptide can inhibit the binding of receptor autoantibodies to a second extracellular loop region of the 5-HT2A receptor by competitive inhibition. In some aspects, the decoy peptide or polypeptide can inhibit the binding of 5-HT2A IgG receptor autoantibodies to a second extracellular loop region of the 5-HT2A receptor. In some aspects, the decoy peptide or polypeptide can inhibit the binding of a 5-HT2A IgG receptor autoantibodies to a second extracellular loop region of the 5-HT2A receptor by competitive inhibition. In some aspects, the decoy peptide or polypeptide can bind to the 5-HT2A receptor autoantibody. In some aspects, the decoy peptide or polypeptide can bind to the 5-HT2A receptor IgG autoantibody,

Disclosed herein are decoy peptides or polypeptides that can comprise fragments of QDDSKVFKEGSCLLADDN (SEQ ID NO: 1). In some aspects, the fragments thereof can comprise a sequence having at least 20%, 30%, 40%, 50%, 60%, 70%, 80%, 90%, 95%, 98%, 99% identity to SEQ ID NO: 1. In some aspects, the fragment or portion retains at least 50%, 75%, 80%, 85%, 90%, 95% or 99% of the biological activity of the reference protein described herein.

In some aspects, the decoy peptides, polypeptides or fragments thereof disclosed herein can comprise a fatty acid moiety. For example, disclosed herein are decoy peptide comprising or consisting of the sequence QDDSKVFKEGSCLLADDN (SEQ ID NO: 1), or a fragment thereof, wherein the decoy peptide further comprises a fatty acid. For example, disclosed herein are decoy peptide comprising or consisting of the sequence QDDSKVFKEGSCLLADDN (SEQ ID NO: 1), or a fragment thereof, wherein the decoy peptide further comprises a fatty acid, wherein the decoy peptide can be:

(SEQ ID NO: 10)
butanoyl-QDDSKVFKEGSCLLADDN;
(SEQ ID NO: 11)
hexanoyl-QDDSKVFKEGSCLLADDN;
(SEQ ID NO: 12)
octanoyl-QDDSKVFKEGSCLLADDN;
(SEQ ID NO: 13)
decanoyl-QDDSKVFKEGSCLLADDN;
(SEQ ID NO: 14)
lauroyl-QDDSKVFKEGSCLLADDN;
(SEQ ID NO: 15)
myristoyl-QDDSKVFKEGSCLLADDN;
(SEQ ID NO: 16)
palmitoyl-QDDSKVFKEGSCLLADDN;
(SEQ ID NO: 17)
stearoyl-QDDSKVFKEGSCLLADDN;
(SEQ ID NO: 18)
palmitoleoyl-QDDSKVFKEGSCLLADDN;
(SEQ ID NO: 19)
arachidoyl-QDDSKVFKEGSCLLADDN;
(SEQ ID NO: 20)
behenoyl-QDDSKVFKEGSCLLADDN;
(SEQ ID NO: 21)
oleoyl-QDDSKVFKEGSCLLADDN;
(SEQ ID NO: 22)
ricinoleoyl-QDDSKVFKEGSCLLADDN;
(SEQ ID NO: 23)
linolenoyl-QDDSKVFKEGSCLLADDN;
(SEQ ID NO: 24)
vacceoyl-QDDSKVFKEGSCLLADDN;
(SEQ ID NO: 25)
gadoleoyl-QDDSKVFKEGSCLLADDN;
(SEQ ID NO: 26)
erucoyl-QDDSKVFKEGSCLLADDN;
(SEQ ID NO: 27)
cetoleoyl-QDDSKVFKEGSCLLADDN;
(SEQ ID NO: 28)
nervonoyl-QDDSKVFKEGSCLLADDN;
(SEQ ID NO: 29)
adrenoyl-QDDSKVFKEGSCLLADDN;
(SEQ ID NO: 30)
α-linolenoyl-QDDSKVFKEGSCLLADDN;
(SEQ ID NO: 31)
γ-linolenoyl-QDDSKVFKEGSCLLADDN;
(SEQ ID NO: 32)
EPA-QDDSKVFKEGSCLLADDN;
(SEQ ID NO: 33)
DHA-QDDSKVFKEGSCLLADDN;
(SEQ ID NO: 34)
butanoyl-SCLLADDN;
(SEQ ID NO: 35)
hexanoyl-SCLLADDN;
(SEQ ID NO: 36)
octanoyl-SCLLADDN;
(SEQ ID NO: 37)
decanoyl-SCLLADDN;
(SEQ ID NO: 38)
lauroyl-SCLLADDN;
(SEQ ID NO: 39)
myristoyl-SCLLADDN;
(SEQ ID NO: 40)
palmitoyl-SCLLADDN;
(SEQ ID NO: 41)
stearoyl-SCLLADDN;
(SEQ ID NO: 42)
palmitoleoyl-SCLLADDN;
(SEQ ID NO: 43)
arachidoyl-SCLLADDN;
(SEQ ID NO: 44)
behenoyl-SCLLADDN;
(SEQ ID NO: 45)
oleoyl-SCLLADDN;
(SEQ ID NO: 46)
ricinoleoyl-SCLLADDN;
(SEQ ID NO: 47)
linolenoyl-SCLLADDN;
(SEQ ID NO: 48)
vacceoyl-SCLLADDN;
(SEQ ID NO: 49)
gadoleoyl-SCLLADDN;
(SEQ ID NO: 50)
erucoyl-SCLLADDN;
(SEQ ID NO: 51)
cetoleoyl-SCLLADDN;
(SEQ ID NO: 52)
nervonoyl-SCLLADDN;
(SEQ ID NO: 53)
adrenoyl-SCLLADDN;
(SEQ ID NO: 54)
α-linolenoyl-SCLLADDN;
(SEQ ID NO: 55)
γ-linolenoyl-SCLLADDN;
(SEQ ID NO: 56)
EPA-SCLLADDN;
(SEQ ID NO: 57)
DHA-SCLLADDN;
(SEQ ID NO: 58)
butanoyl-QDDSKVF;
(SEQ ID NO: 59)
hexanoyl-QDDSKVF;
(SEQ ID NO: 60)
octanoyl-QDDSKVF;
(SEQ ID NO: 61)
decanoyl-QDDSKVF;
(SEQ ID NO: 62)
lauroyl-QDDSKVF;
(SEQ ID NO: 63)
myristoyl-QDDSKVF;
(SEQ ID NO: 64)
palmitoyl-QDDSKVF;
(SEQ ID NO: 65)
stearoyl-QDDSKVF;
(SEQ ID NO: 66)
palmitoleoyl-QDDSKVF;
(SEQ ID NO: 67)
arachidoyl-QDDSKVF;
(SEQ ID NO: 68)
behenoyl-QDDSKVF;
(SEQ ID NO: 69)
oleoyl-QDDSKVF;
(SEQ ID NO: 70)
ricinoleoyl-QDDSKVF;
(SEQ ID NO: 71)
linolenoyl-QDDSKVF;
(SEQ ID NO: 72)
vacceoyl-QDDSKVF;
(SEQ ID NO: 73)
gadoleoyl-QDDSKVF;
(SEQ ID NO: 74)
erucoy1-QDDSKVF;
(SEQ ID NO: 75)
cetoleoyl-QDDSKVF;
(SEQ ID NO: 76)
nervonoyl-QDDSKVF;
(SEQ ID NO: 77)
adrenoyl-QDDSKVF;
(SEQ ID NO: 78)
α-linolenoyl-QDDSKVF;
(SEQ ID NO: 79)
γ-linolenoyl-QDDSKVF;
(SEQ ID NO: 80)
EPA-QDDSKVF;
(SEQ ID NO: 81)
DHA-QDDSKVF;
(SEQ ID NO: 82)
butanoyl-VFKEGSC;
(SEQ ID NO: 83)
hexanoyl-VFKEGSC;
(SEQ ID NO: 84)
octanoyl-VFKEGSC;
(SEQ ID NO: 85)
decanoyl-VFKEGSC;
(SEQ ID NO: 86)
lauroyl-VFKEGSC;
(SEQ ID NO: 87)
myristoyl-VFKEGSC;
(SEQ ID NO: 88)
palmitoyl-VFKEGSC;
(SEQ ID NO: 89)
stearoyl-VFKEGSC;
(SEQ ID NO: 90)
palmitoleoyl-VFKEGSC;
(SEQ ID NO: 91)
arachidoyl-VFKEGSC;
(SEQ ID NO: 92)
behenoyl-VFKEGSC;
(SEQ ID NO: 93)
oleoyl-VFKEGSC;
(SEQ ID NO: 94)
ricinoleoyl-VFKEGSC;
(SEQ ID NO: 95)
linolenoyl-VFKEGSC;
(SEQ ID NO: 96)
vacceoyl-VFKEGSC;
(SEQ ID NO: 97)
gadoleoyl-VFKEGSC;
(SEQ ID NO: 98)
erucoyl-VFKEGSC;
(SEQ ID NO: 99)
cetoleoyl-VFKEGSC;
(SEQ ID NO: 100)
nervonoyl-VFKEGSC;
(SEQ ID NO: 101)
adrenoyl-VFKEGSC;
(SEQ ID NO: 102)
α-linolenoyl-VFKEGSC;
(SEQ ID NO: 103)
γ-linolenoyl-VFKEGSC;
(SEQ ID NO: 104)
EPA-VFKEGSC;
or
(SEQ ID NO: 105)
DHA-VFKEGSC.

In the foregoing, the fatty acid moiety is shown at the left side and is linked to the peptide QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4). “EPA” indicates a moiety derived from 5,8,11,14,17-eicosapentaenoic acid; and “DHA” indicates a moiety derived from 4,7,10,13,16,19-docosahexaenoic acid.

In some instances, the decoy peptide, polypeptide or fragment thereof can be any of the disclosed decoy peptides, polypeptidse or fragments thereof comprising an acetylated fatty acid.

Exemplary fatty acids from which a fatty acid moiety is derived include, without limitation, butyric acid, caproic acid, caprylic acid, capric acid, decanoic acid, lauric acid, myristic acid, palmitic acid, pentadecanoic acid, stearic acid, arachidic acid, behenic acid, erucic acid, lignoceric acid, margaric acid, myristoleic acid, palmitoleic acid, oleic acid, gadoleic acid, ricinoleic acid, vaccenic acid, linoleic acid, linolenic acid, alpha-linolenic acid, gamma-linolenic acid, licanic acid, margaroleic acid, arachidic acid, gadoleic acid, nervonic acid, arachidonic acid, docosapentaenoic (DPA), eicosapentaenoic acid (EPA), docosahexaenoic acid (DHA), and the like.

Described herein is an unexpected effect using a myrsitolated decoy peptide. It was observed that a single IP injection of Myristolated Peptide 2 (SEQ ID NO: 9) significantly lowered blood pressure for an unexpectedly long duration. For instance, seven and/or eight days following a single IP injection of Myristolated Peptide 2 (SEQ ID NO: 9) a persistent and significant systolic and diastolic blood pressure-lowering effect was observed compared to the respective baseline levels

In some instances, the decoy peptide can be any of the disclosed peptides comprising a saturated fatty acid. Exemplary saturated fatty acids include, but are not limited to, propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, and hexatriacontanoic acid.

In some instances, the decoy peptide can be any of the disclosed peptides comprising a unsaturated fatty acid. Exemplary unsaturated fatty acids include, but are not limited to, myristoleic acid, palmitoleic acid, sapienic acid, oleic acid, linoleic acid, α-linolenic acid, arachidonic acid, eicosapentaenoic acid (EPA), erucic acid, docosahexaenoic acid (DHA), and docosapentaenoic acid.

The decoy peptides and polypeptides disclosed herein can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

In some aspects, any of SCLLADDN (SEQ ID NO: 2), QDDSKVF (SEQ ID NO: 3), or VFKEGSC (SEQ ID NO: 4) can be conjugated to a fatty acid. In some aspects, SCLLADDN (SEQ ID NO: 2), QDDSKVF (SEQ ID NO: 3), or VFKEGSC (SEQ ID NO: 4) can be conjugated to myristic acid. For example, myristic acid can be conjugated to SEQ ID NO: 2 to form myristoyl-SCLLADDN (SEQ ID NO: 9).

Disclosed herein are decoy peptides or polypeptides that can comprise variants of QDDSKVFKEGSCLLADDN (SEQ ID NO: 1). In some aspects, the variants can comprise a sequence having at least 20%, 30%, 40%, 50%, 60%, 70%, 80%, 90%, 95%, 98%, 99% identity to SEQ ID NO: 1. In some aspects, the variants retains at least 50%, 75%, 80%, 85%, 90%, 95% or 99% of the biological activity of the reference protein described herein.

Disclosed herein are decoy peptides or polypeptides that can comprise variants of SCLLADDN (SEQ ID NO: 2), QDDSKVF (SEQ ID NO: 3), or VFKEGSC (SEQ ID NO: 4). In some aspects, the variants can comprise a sequence having at least 20%, 30%, 40%, 50%, 60%, 70%, 80%, 90%, 95%, 98%, 99% identity to SEQ ID NO: 1, SEQ ID NO: 2, SEQ ID NO: 3 or SEQ ID NO: 4. In some aspects, the variants retains at least 50%, 75%, 80%, 85%, 90%, 95% or 99% of the biological activity of the reference protein described herein.

In some aspects, the decoy peptide or polypeptide can be of any length so long as the binding of the 5-HT2A receptor autoantibodies to the second extracellular loop region of the receptor is blocked or inhibited.

In some aspects, the decoy peptides, polypeptides or fragments thereof described herein can further comprise 1, 2, 3, 4, 5, 10, 15, 20, 25, 30, 35, 40, 45, 50 amino acid residues at the N-terminal end of the disclosed decoy peptides, polypeptides or fragments thereof. In some aspects, the decoy peptides, polypeptides or fragments thereof described herein can further comprise 1, 2, 3, 4, 5, 10, 15, 20, 25, 30, 35, 40, 45, 50 amino acid residues at the C-terminal end of the disclosed decoy peptides, polypeptides or fragments thereof disclosed herein. In some aspects, the amino acid residues that can be present at either the N-terminal end or the C-terminal end of any of the decoy peptides, polypeptides or fragments thereof disclosed herein can be unimportant for inhibiting the binding of the 5-HT2A receptor autoantibodies which bind to the second extracellular loop region of the 5-HT2A receptor. In some aspects, the amino acid residues added to the N-terminal end or the C-terminal end of the decoy peptides, polypeptides or fragments thereof disclosed herein may prevent ubiquitination, improve stability, help maintain the three dimensional structure of the peptide, or a combination thereof.

In some aspects, the decoy peptides, polypeptides or fragments thereof disclosed herein can further comprise a peptide or polypeptide having one or more amino acid residues with a modified side chain. In some aspects, one or more amino acids of any of the decoy peptides, polypeptides or fragments thereof disclosed here can have a modified side chain. Examples of side chain modifications include but are not limited to modifications of amino acid groups, such as reductive alkylation; amidination with methylacetimidate; acylation with acetic anhydride; carbamolyation of amino groups with cynate; trinitrobenzylation of amino acid with 2,4,6-trinitrobenzene sulfonic acid (TNBS); alkylation of amino groups with succinic anhydride; and pyridoxylation with pridoxal-5-phosphate followed by reduction with NaBH₄.

In some aspects, the guanidine group of the arginine residue may be modified by the formation of a heterocyclic condensate using a reagent, such as 2,3-butanedione, phenylglyoxal, and glyoxal. In some aspects, the carboxyl group may be modified by carbodiimide activation via O-acylisourea formation, followed by subsequent derivatization, for example, to a corresponding amide.

In some aspects, the sulfhydryl group may be modified by methods, such as carboxymethylation with iodoacetic acid or iodoacetamide; performic acid oxidation with cysteic acid; formation of mixed disulfides by other thiol compounds; a reaction by maleimide, maleic anhydride, or other substituted maleimide; formation of mercury derivatives using 4-chloromercuribenzoate, 4-chloromercuriphenylsulfonic acid, phenylmercury chloride, 2-chloromercuri-4-nitrophenol, and other mercurial agents; and carbamolyation with cyanate at alkaline pH. In addition, the sulfhydryl group of cysteine may be substituted with a selenium equivalent, whereby a diselenium bond may be formed instead of at least one disulfide bonding site in the peptide.

In some aspects, the tryptophan residue may be modified by, for example, oxidation with N-bromosuccinimide or alkylation of the indole ring by 2-hydroxy-5-nitrobenzyl bromide or sulfonyl halide. Meanwhile, the tyrosine residue may be modified by nitration using tetranitromethane to form a 3-nitrotyrosine derivative.

In some aspects, the modification of the imidazole ring of the histidine residue may be accomplished by alkylation with an iodoacetic acid derivative or N-carbethoxylation with diethylpyrocarbonate.

In some aspects, the proline residue may be modified by, for example, hydroxylation at the 4-position.

In some aspects, decoy peptides (including any fragments or variants thereof) disclosed herein can further comprise at least one polyethylene glycolated (PEGylated) group attached to the N-terminus. In some aspects, the pegylated decoy peptides have can a longer half-life of the non-pegylated decoy peptides. In some aspects, the pegylated decoy peptides can have a half-life of at least 30 hours. In some aspects, the pegylated decoy peptides can have a half-life of 30, 31, 32, 33, 34, 35, 36, 37, 38, 39, 40, 41, 42, 43, 44, 45, 46, 46, 47, 48 hours or longer.

Polyethylene Glycol (PEG) is a well-known polymer with good solubility in many aqueous and organic solvents, which exhibits low toxicity, lack of immunogenicity, and is clear, colorless, odorless, and stable. For these reasons and others, PEG has been selected as the preferred polymer for attachment, but it has been employed solely for purposes of illustration and not limitation. Similar products may be obtained with other water-soluble polymers, including without limitation; polyvinyl alcohol, other poly(alkylene oxides) such as poly(propylene glycol) and the like, poly(oxyethylated polyols) such as poly(oxyethylated glycerol) and the like, carboxymethylcellulose, dextran, polyvinyl alcohol, polyvinyl purrolidone, poly-1,3-dioxolane, poly-1,3,6-trioxane, ethylene/maleic anhydride, and polyaminoacids. One skilled in the art will be able to select the desired polymer based on the desired dosage, circulation time, resistance to proteolysis, and other considerations.

Representative polymeric reagents and methods for conjugating such polymers to an active moiety are described in Harris, J. M. and Zalipsky, S., Eds, Poly(ethylene glycol), Chemistry and Biological Applications, ACS, Washington, 1997; Veronese, F., and J. M. Harris, Eds., Peptide and Protein PEGylation, Advanced Drug Delivery Reviews, 54(4); 453-609 (2002); Zalipsky, S., et al., “Use of Functionalized Poly Ethylene Glycols) for Modification of Polypeptides” in Polyethylene Glycol Chemistry: Biotechnical and Biomedical Applications, J. M. Harris, ed., Plenus Press, New York (1992); Zalipsky (1995) Advanced Drug Reviews 16:157-182; and in Roberts et al., Adv. Drug Delivery Reviews, 54, 459-476 (2002).

A wide variety of PEG derivatives are both commercially available and suitable for use in the preparation of the PEG-conjugates of the invention. For example, NOF Corp.'s SUNBRIGHTO Series (peg-drug.com) provides numerous PEG derivatives, including methoxypolyethylene glycols and activated PEG derivatives such as succinimidyl ester, methoxy-PEG amines, maleimides, and carboxylic acids, for coupling by various methods to C-peptide and Nektar Therapeutics' Advanced PEGylation also offers diverse PEG-coupling technologies to improve the safety and efficacy of therapeutics. Additional PEGs for use in forming a C-peptide conjugate of the invention include those available from Polypure (Norway), from QuantaBioDesign LTD (Ohio) and Sunbio, Inc (South Korea). Further PEG reagents suitable for use in forming a conjugate of the invention, and methods of conjugation are described in the Pasut. G., et al., Expert Opin. Ther. Patents (2004), 14(6) 859-893.

The PEGylated decoy peptides described herein can have PEG moieties with a molecular weight varying within a range of about 4,000 Da to 80,000 Da. The molecular weight ranges will typically be from about 4000 Da to about 10,000 Da, from about 10,000 Da to about 20,000 Da, from about 20,000 Da to about 30,000 Da, from about 30,000 Da to about 40,000 Da, from about 40,000 Da to about 50,000 Da, from about 50,000 Da to about Da, from about 60,000 Da to about 70,000 Da, and from about 70,000 Da to about Da. Non-limiting examples of average molecular weights of the PEG moieties are about 10,000 Da, about 20,000 Da, about 30,000 Da, about 40,000 Da, about 50,000 Da, about 60,000 Da, about 70,000 Da, and about 80,000 Da.

Because virtually all PEG polymers exist as mixtures of diverse high molecular mass, PEG molecular weight (MW) is typically reported as number average (Mn), weight average (Mw), or z-average (Mz) molecular weights. The weight average is probably the most useful of the three, because it fairly accounts for the contributions of different sized chains to the overall behavior of the polymer, and correlates best with most of the physical properties of interest.

The PEG groups will for a given molecular weight typically consist of a range of ethylene glycol (or ethyleneoxide; OCH2CH2) monomers. For example, a PEG group of molecular weight 2000 Da will typically consist of 43±10 monomers, the average being around 43-44 monomers.

The PEG groups will typically comprise a number of subunits, e.g., each n, n1 or n2 or n3 in any of the claimed compounds may each independently be from about 1 to about 1000, from about 1 to about 800, from about 1 to about 600, from about 1 to about 400, from about 1 to about 300, from about 1 to about 200. Well-suited PEG groups are such wherein the number of subunits (i.e., n1, n2, and n3) are independently selected from the group consisting of from about 800 to about 1000; from about 800 to about 950; from about 600 to about 850; from about 400 to about 650; from about 200 to about 450, from about 180 to about 350; from about 100 to about 150; from about 35 to about 55; from about 42 to about 62; from about 12 to about 25 subunits, from about 1 to 10 subunits. In aspects, the PEGylated decoy peptides will have a molecular weight of about 40 kDa, and thus n1 and n2 for each PEG chain in the branch chain PEGs will be within the range of about 440 to about 550, or about 450 to about 520.

Branched versions of the PEG polymer (e.g., a branched 40,000 Da PEG polymer comprised of two or more 10,000 Da to 20,000 Da PEG polymers or the like) having a total molecular weight of any of the foregoing can also be used.

Representative branched polymers described therein include those having the following generalized structure: (PEG)y-[Core]-[Linker]; where “[Core]” is a central or core molecule from which extends 2 or more PEG arms, the variable “y” represents the number of PEG arms, and “[Linker]” represents an optional linking moiety (as further defined herein) that typically couples the [Core] to the decoy peptide. In some aspects of the branched chain PEGs, at least one polymer arm possesses a terminal functional group suitable (e.g. NHS moiety) for reaction with decoy peptide. Typically the branched chain polymers of the invention are coupled to the N-terminal amino group of the decoy peptide.

In some aspects, the linker moiety can represent either a hydrolytically stable, or alternatively, a degradable linker, meaning that the linkage can be hydrolyzed under physiological conditions, e.g., the linkage comprises an ester, hydrolysable carbamate, carbonate, or other such group. Hydrolytically degradable linkages, useful not only as a degradable linkage within a polymer backbone, but also, in the case of certain embodiments of the invention, for covalently attaching a water-soluble polymer to a decoy peptide, include: carbonate; imine resulting, for example, from reaction of an amine and an aldehyde (see, e.g., Ouchi et al. (1997) Polymer Preprints 38(1):582-3); phosphate ester, formed, for example, by reacting an alcohol with a phosphate group; hydrazone, e.g., formed by reaction of a hydrazide and an aldehyde; acetal, e.g., formed by reaction of an aldehyde and an alcohol; orthoester, formed, for example, by reaction between a formate and an alcohol; and esters, and certain urethane (carbamate) linkages. Illustrative PEG reagents for use in preparing a releasable decoy peptide conjugate in accordance with the invention are described in U.S. Pat. Nos. 6,348,558, 5,612,460, 5,840,900, 5,880,131, and 6,376,470. Typically releasable linkers may be attached to any residue in decoy peptide, and are not restricted to the N-terminal amino acid.

Branched PEGS such as those represented generally by the formula, (PEG)y-[Core]-[Linker], above can possess 2 polymer arms to about 8 polymer arms (i.e., “y” ranges from 2 to about 8). Preferably, such branched PEGs typically possess from 2 to about 4 polymer arms, Multi-armed polymers include those having 2, 3, 4, 5, 6, 7 or 8 PEG arms.

Core molecules in branched PEGs as described herein include polyols, which are then further functionalized. Such polyols include aliphatic polyols having from 1 to 10 carbon atoms and from 1 to 10 hydroxyl groups, including ethylene glycol, alkane diols, alkyl glycols, alkylidene alkyl diols, alkyl cycloalkane diols, 1,5-decalindiol, 4,8-bis(hydroxymethyl)tricyclodecane, cycloalkylidene diols, dihydroxyalkanes, trihydroxyalkanes, and the like. Cycloaliphatic polyols may also be employed, including straight chained or closed-ring sugars and sugar alcohols, such as mannitol, sorbitol, inositol, xylitol, quebrachitol, threitol, arabitol, erythritol, adonitol, ducitol, facose, ribose, arabinose, xylose, lyxose, rhamnose, galactose, glucose, fructose, sorbose, mannose, pyranose, altrose, talose, tagitose, pyranosides, sucrose, lactose, maltose, and the like. Additional aliphatic polyols include derivatives of glyceraldehyde, glucose, ribose, mannose, galactose, and related stereoisomers. Other core polyols that may be used include crown ether, cyclodextrins, dextrins and other carbohydrates such as starches and amylose. Typical polyols include glycerol, pentaerythritol, sorbitol, and trimethylolpropane. Other suitable cores include lysine, and other polyamines, and PEG moieties comprising multiple functional terminal end groups.

Illustrative multi-armed PEGs having 2 arms, 3 arms, 4 arms, and 8 arms are known in the art, and are available commercially and/or can be prepared following techniques known to those skilled in the art. (See generally Pasut et al., (2004) Protein, peptide and non-peptide drug PEGylation for therapeutic application Expert Opinin. Ther. Patents 14(6) 859-894). Additional branched-PEGs for use in forming a C-peptide conjugate of the present invention include those described in U.S. Patent Application Publication Nos. 20050009988, 20060194940, 20090234070, 20070031371, U.S. Pat. Nos. 6,664,331; 6,362,254; 6,437,025; 6,541,543; 6,664,331; 6,730,334; 6,774,180; 6,838,528; 7,030,278; 7,026,440; 7,053,150; 7,157,546; 7,223,803; 7,265,186; 7,419,600; 7,432,330; 7,432,331; 7,511,094; 7,528,202; 7,589,157; and PCT publication numbers WO2005000360, WO2005108463, WO2005107815, WO2005028539 and WO200605108463.

In some aspects, the decoy peptides, polypeptides, or fragments thereof can comprise PEG and a fatty acid. In some aspects, the PEG can be conjugated to the decoy peptide, polypeptide, or fragment thereof at the C-terminal end, and the fatty acid can be conjugated to the decoy peptide, polypeptide, or fragment thereof at the N-terminal end. In some aspects, the PEG can be conjugated to the decoy peptide, polypeptide, or fragment thereof at the N-terminal end, and the fatty acid can be conjugated to the decoy peptide, polypeptide, or fragment thereof at the C-terminal end.

In some aspects, the decoy peptides, polypeptides or fragments thereof described herein can be further modified to improve stability. In some aspects, any of the amino acid residues of the decoy peptides, polypeptides or fragments thereof described herein can be modified to improve stability. In some aspects, decoy peptide or polypeptide can have at least one amino acid residue that has an acetyl group, a fluorenylmethoxy carbonyl group, a formyl group, a palmitoyl group, a myristyl group, a stearyl group, or polyethylene glycol. In some aspects, an acetyl protective group can be bound to the decoy peptide or polypeptide described herein.

As used herein, the term “stability” refers to storage stability (e.g., room-temperature stability) as well as in vivo stability. The foregoing protective group can protect the decoy peptides, polypeptides or fragments thereof described herein from the attack of protein cleavage enzymes in vivo.

As used herein, the terms “decoy peptide or polypeptide” and “decoy peptides, polypeptides or fragments thereof” can also be used to include functional equivalents of the decoy peptides or polypeptides and the decoy peptides, polypeptides or fragments thereof described herein. As used herein, the term “functional equivalents” can refer to amino acid sequence variants having an amino acid substitution, addition, or deletion in some of the amino acid sequence of the decoy peptide or polypeptide while simultaneously having similar or improved biological activity, compared with the decoy peptide or polypeptide as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met). In some aspects, the amino acid deletion can be located in a region that is not directly involved in the activity of the decoy peptide and polypeptide disclosed herein.

In some aspects, the amino acid sequence of the decoy peptides, polypeptides or fragments thereof described herein can include a peptide sequence that has substantial identity to any of the sequences of the decoy peptides, polypeptides or fragments thereof disclosed herein. As used herein, the term “substantial identity” means that two amino acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the amino acid sequence of the decoy peptides, polypeptides or fragments thereof described herein can include a peptide sequence that has some degree of identity or homology to any of sequences of the decoy peptides, polypeptides or fragments thereof disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The decoy peptides or polypeptides described herein can have at least or about 25%, 50%, 65%, 75%, 80%, 85%, 90%, 95%, 96%, 97%, 98%, or 99% identity or homology to the decoy peptide or polypeptide, wherein the decoy peptide or polypeptide is one or more of SEQ ID NOs: 1-4.

In some aspects, the decoy peptides, polypeptides or fragments thereof described herein can be further conjugated to an Fc fragment of immunoglobulin G. In some aspects, the Fc fragment of immunoglobulin G can bind to (and remove) bound 5-HT2A receptor autoantibodies on cells.

In some aspects, the decoy peptides, polypeptides or fragments thereof described herein can be part of a scaffold protein. Disclosed herein are scaffold proteins comprising: a SH3 (src homolog3), SH2 (src homolog2), PDZ, or GTPase-binding domain (GBD) and a heterologous peptide. In some aspects, the heterologous peptide can be inserted or connected to one or more of a SH3 (src homolog3), SH2 (src homolog2), PDZ, or GTPase-binding domain (GBD). In some aspects, the heterologous peptide can be one or more of the decoy peptides, polypeptides or fragments thereof disclosed herein. In some aspects, the heterologous peptide can comprise or consist of one or more decoy peptides comprising the sequence of QDDSKVFKEGSCLLADDN (SEQ ID NO: 1), or a fragment thereof, SCLLADDN (SEQ ID NO: 2), QDDSKVF (SEQ ID NO: 3), or VFKEGSC (SEQ ID NO: 4).

In some aspects, the heterologous peptide can comprise or consist of one or more decoy peptides comprising a variant of the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4). In some aspects, the scaffold protein is capable of inhibiting the binding of 5-HT2A receptor autoantibodies to a second extracellular loop region of the 5-HT2A receptor. In some aspects, the scaffold protein can inhibit the binding of 5-HT2A receptor autoantibodies to a second extracellular loop region of the 5-HT2A receptor by competitive inhibition. In some aspects, the heterologous peptide of the scaffold can bind to PDZ via the carboxy terminal region of the decoy peptide or polypeptide. In some aspects, the heterologous peptide of the scaffold can be bound to SH3, SH2 or PDZ via a linker. In some aspects, the linker can be positioned between SH3, SH2 or PDZ and the N-terminus of the heterologous peptide. In some aspects, the linker can be designed to comprise the consensus motif of SH3, SH2 or PDZ. For example, PDZ targets the consensus motif Glu-(Ser/Thr)-Xaa-Val/Ile (SEQ ID NO: 8), wherein Xaa can represent any amino acid. SH3, SH2, PDZ or GBD can bind to the linker-heterologous peptide via the condense motif present in the linker.

Disclosed herein are compositions comprising one or more of the decoy peptides, polypeptides or fragments thereof described herein capable of lowering plasma triglycerides in a subject, lowering alanine transaminase (ALT) in a subject, identifying a subject at risk of developing hypertriglyceridemia, identifying a subject at risk of elevated levels of alanine aminotransferase (ALT), treating a subject at risk of developing hypertriglyceridemia, treating hypertriglyceridemia in a subject, treating obesity in a subject, treating diabetic dyslipidemia in a subject, treating premature atherosclerosis in a subject, treating atherosclerotic heart disease in a subject, treating a subject at risk for having a stroke or preventing or ameliorating a symptom of stroke in a subject, treating hypertension in a subject, treating fatty liver disease in a subject, reducing the risk of developing liver cirrhosis or liver failure in a subject, preventing cardiac steatosis in a subject, or a combination thereof. In some aspects, the subject has type 2 diabetes. In some aspects, the subject is obese. In some aspects, the subject the subject has type 2 diabetes, a neurologic disease or disorder, a microvascular disease or disorder, hypertension, schizophrenia, retinitis pigmentosa, mild cognitive dysfunction, primary open angle glaucoma, diabetic dyslipidemia, fatty liver disease, type 2 hyperlipidemia, hypertriglyceridemia, microvascular angiopathy, diabetic kidney disease, Parkinson's disease, dementia, major depressive disorder, obesity, refractory hypertension, essential hypertension or has had a stroke or a traumatic brain injury, is overweight, or a combination thereof. In some aspects, the subject the subject does not have or has not been diagnosed with type 2 diabetes, a neurologic disease or disorder, a microvascular disease or disorder, hypertension, schizophrenia, retinitis pigmentosa, mild cognitive dysfunction, primary open angle glaucoma, diabetic dyslipidemia, fatty liver disease, type 2 hyperlipidemia, hypertriglyceridemia, microvascular angiopathy, diabetic kidney disease, Parkinson's disease, dementia, major depressive disorder, obesity, refractory hypertension, essential hypertension or has had a stroke or a traumatic brain injury, is not overweight, or a combination thereof.

Disclosed herein are compositions comprising one or more of the decoy peptides, polypeptides or fragments thereof described herein capable of preventing or reducing the risk of a neurologic disease or a microvascular disease in a subject. In some aspects, the subject has type 2 diabetes, microvascular angiopathy, diabetic kidney disease, Parkinson's disease, dementia, major depressive disorder, obesity, refractory hypertension, essential hypertension or has had a stroke or a traumatic brain injury or a combination thereof. In some aspects, the subject has type 2 diabetes. In some aspects, the subject does not have or has not been diagnosed with type 2 diabetes, microvascular angiopathy, diabetic kidney disease, Parkinson's disease, dementia, major depressive disorder, obesity, refractory hypertension, essential hypertension or has had a stroke or a traumatic brain injury or a combination thereof. In some aspects, the compositions disclosed herein are capable of preventing or reducing cognitive decline (cognitive dysfunction including changes in memory), mood, or one or symptoms associated with depression in a subject. In some aspects, the subject has type 2 diabetes and a traumatic brain injury. In some aspects, the subject was diagnosed with type 2 diabetes before the traumatic brain injury. In some aspects, the subject has a traumatic brain injury. In some aspects, the subject does not have or has not been diagnosed with type 2 diabetes.

Disclosed herein are compositions comprising one or more of the decoy peptides, polypeptides or fragments thereof described herein capable of reducing or preventing or reducing cardiac hypertrophy in a subject. In some aspects, the subject has type 2 diabetes, microvascular angiopathy, diabetic kidney disease, Parkinson's disease, dementia, major depressive disorder, obesity, refractory hypertension, essential hypertension or has had a stroke or a traumatic brain injury or a combination thereof. In some aspects, the subject has type 2 diabetes. In some aspects, the hypertension can be moderate or severe hypertension. In some aspects, the subject does not have or has not been diagnosed with type 2 diabetes, microvascular angiopathy, diabetic kidney disease, Parkinson's disease, dementia, major depressive disorder, obesity, refractory hypertension, essential hypertension or has had a stroke or a traumatic brain injury or a combination thereof. In some aspects, the compositions disclosed herein are capable of preventing or reducing cognitive decline (cognitive dysfunction including changes in memory), mood, or one or symptoms associated with depression in a subject. In some aspects, the subject has type 2 diabetes and a traumatic brain injury. In some aspects, the subject was diagnosed with type 2 diabetes before the traumatic brain injury. In some aspects, the subject has a traumatic brain injury. In some aspects, the subject does not have or has not been diagnosed with type 2 diabetes. In some aspects, the subject has or was diagnosed with hypertension.

Pharmaceutical Compositions

As disclosed herein, are pharmaceutical compositions, comprising one or more of the decoy peptides, polypeptides or fragments thereof described herein. Also disclosed herein, are pharmaceutical compositions, comprising one or more of the decoy peptides, polypeptides or fragments thereof described herein and a pharmaceutical acceptable carrier. Further disclosed herein are pharmaceutical compositions comprising one or more of the decoy peptides, polypeptides or fragments thereof described herein for lowering plasma triglycerides in a subject, lowering alanine transaminase (ALT) in a subject, reducing or preventing cardiac hypertrophy in a subject, identifying a subject at risk of developing hypertriglyceridemia, identifying a subject at risk of elevated levels of alanine aminotransferase (ALT), treating a subject at risk of developing hypertriglyceridemia, treating hypertriglyceridemia in a subject, treating obesity in a subject, treating diabetic dyslipidemia in a subject, treating premature atherosclerosis in a subject, treating atherosclerotic heart disease in a subject, treating a subject at risk for having a stroke or preventing or ameliorating a symptom of stroke in a subject, treating hypertension in a subject, treating fatty liver disease in a subject, reducing the risk of developing liver cirrhosis or liver failure in a subject, preventing cardiac steatosis in a subject, or a combination thereof. Further disclosed herein are pharmaceutical compositions comprising one or more of the decoy peptides, polypeptides or fragments thereof described herein for reducing or preventing acute neurite retraction or preventing accelerated neuron loss. Also disclosed herein are pharmaceutical compositions comprising one or more of the decoy peptides, polypeptides or fragments thereof described herein for reducing the risk of a neurologic disease or disorder or microvascular disease or disorder or a neurodegenerative disease or disorder. In some aspects, the pharmaceutical compositions can comprise: a) a therapeutically effective amount of one or more of the decoy peptides, polypeptides or fragments thereof described herein; and b) a pharmaceutically acceptable carrier. For example, described herein are pharmaceutical compositions comprising: a) a therapeutically effective amount of a decoy peptide comprising or consisting of the sequence QDDSKVFKEGSCLLADDN (SEQ ID NO: 1), or a fragment thereof and b) a pharmaceutically acceptable carrier. In some aspects, the decoy peptides described herein can inhibit the binding of 5-HT2A receptor autoantibodies to a second extracellular loop region of the 5-HT2A receptor. In some aspects, the decoy peptides described herein can inhibit the binding of 5-HT2A receptor autoantibodies to a second extracellular loop region of the 5-HT2A receptor by competitive inhibition. In some aspects, the decoy peptide can be pegylated. In some aspects, the decoy peptide further comprises at least one polyethylene glycolated (PEGylated) group attached to the N-terminus.

The pharmaceutical compositions described above can be formulated to include a therapeutically effective amount of one or more of the decoy peptides of polypeptides described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of neurologic disease or disorder or microvascular disease or disorder, or to any of the diseases, disorders or conditions including but not limited to type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia or has had a stroke or a traumatic brain injury, being overweight, or a combination thereof.

The pharmaceutical compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with type 2 diabetes (or to a subject that is not diagnosed with type 2 diabetes or to a subject that does not have type 2 diabetes) in an amount sufficient to at least partially improve a sign or symptom or to inhibit the progression of (and preferably arrest) the symptoms of the condition, its complications, and consequences (e.g., developing a neurologic disease or disorder or microvascular disease or disorder or neurodegenerative disease or disorder). In some aspects, the compositions can be administered to a subject (e.g., a human patient) already with or diagnosed with a traumatic brain injury with or without a diagnosis of type 2 diabetes in an amount sufficient to at least partially improve a sign or symptom or to inhibit the progression of (and preferably arrest) the symptoms of the condition, its complications, and consequences (e.g., developing a neurologic disease or disorder or microvascular disease or disorder or a neurodegenerative disorder). In some aspects, the compositions can be administered to a subject (e.g., a human patient) already having or diagnosed with hypertension in an amount sufficient to at least partially improve a sign or symptom or to inhibit the progression of (and preferably arrest) the symptoms of the condition, its complications, and consequences (e.g., cardiac hypertrophy).

The compositions and pharmaceutical compositions comprising one or more of the decoy peptides, polypeptides or fragments thereof described herein can be administered to a subject (e.g., a human patient) already with or diagnosed with type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia or has had a stroke or a traumatic brain injury or a combination thereof. In some aspects, the compositions or pharmaceutical compositions can be administered to a subject (e.g., a human patient) already with or diagnosed with type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia or has had a stroke or a traumatic brain injury or a combination thereof in an amount sufficient to at least partially improve a sign or symptom or to inhibit the progression of (and preferably arrest) the symptoms of the condition, its complications, and consequences (e.g., developing hypertriglyceridemia, diabetic dyslipidemia, premature atherosclerosis, atherosclerotic heart disease, having a stroke, cardiac steatosis, fatty liver disease, liver cirrhosis and/or liver failure.). An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of acute neurite retraction, accelerated neuron loss, a neurologic disease or disorder or microvascular disease or disorder or neurodegenerative disease or disorder or a symptom of the acute neurite retraction, accelerated neuron loss, a neurologic disease or disorder or microvascular disease or disorder or neurodegenerative disease or disorder is ameliorated or or cardiac hypertrophy is reduced or prevented. Further, a therapeutically effective amount also includes amounts that provide a treatment in which the onset or progression of type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, being overweight, diabetic dyslipidemia, hypertriglyceridemia, type 2B hyperlipidemia, premature atherosclerosis, atherosclerotic heart disease, fatty liver disease, liver cirrhosis, liver failure, cardiac steatosis, a stroke, a traumatic brain injury or a combination thereof is ameliorated. One or more of the symptoms can be less severe. In some aspects, recovery can be accelerated in an individual who has been treated with one or more of the compositions or pharmaceutical compositions disclosed herein.

In some aspects, the pharmaceutical composition can be formulated for intravenous administration. In some aspects, the pharmaceutical composition can be formulated for subcutaneous, intranasal, intramuscular or oral administration. In some aspects, the compositions of the present disclosure also contain a therapeutically effective amount of a 5-HT2A receptor antagonist. In some aspects, the 5-HT2A receptor antagonist can be a peptide. In some aspects, the 5-HT2A receptor antagonist can be ketanserin or volinanserin (also known as MDL-100,907). The compositions or pharmaceutical compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the decoy peptide. Thus, compositions can be prepared for parenteral administration that includes the decoy peptides, polypeptides or fragments thereof dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting pharmaceutical compositions in solid form can be packaged in multiple single dose units, each containing a fixed amount of the above-mentioned agent or agents, such as in a sealed package of tablets or capsules. The pharmaceutical composition in solid form can also be packaged in a container for a flexible quantity, such as in a squeezable tube designed for a topically applicable cream or ointment.

Methods of Treatment

Disclosed herein are methods of lowering plasma triglycerides in a subject. In some aspects, the methods can comprise: administering to a subject a therapeutically effective amount of one or more decoy peptides, polypeptides or fragments thereof disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of lowering alanine transaminase (ALT) in a subject. In some aspects, the methods can comprise: administering to a subject a therapeutically effective amount of one or more decoy peptides, polypeptides or fragments thereof disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Also disclosed herein are methods of identifying a subject having or at risk for or developing hypertriglyceridemia. In some aspects, the subject has type 2 diabetes, essential hypertension, or obesity. In some aspects, the subject does not have or has not been diagnosed with type 2 diabetes, essential hypertension or obesity. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); and d) identifying the subject having or at risk for developing hypertriglyceridemia when the level of binding in step c) is higher when compared to the level of binding in a reference sample. In some aspects, the methods can comprise: a) obtaining or having obtained a sample from the subject, wherein the sample can comprise one or more 5-HT2A receptor autoantibodies; b) contacting the sample of step a) with one or more decoy peptides comprising or consisting of the sequence of QDDSKVFKEGSCLLADDN (SEQ ID NO: 1) or a fragment thereof; c) determining the level of binding of the 5-HT2A receptor autoantibodies to the one or more decoy peptides comprising or consisting of the sequence of QDDSKVFKEGSCLLADDN (SEQ ID NO: 1) or a fragment thereof; and d) identifying the subject having or at risk for developing hypertriglyceridemia when the level of binding in step c) is higher when compared to the level of binding in a reference sample. In some aspects, the method can further comprise administering to the subject a therapeutically effective amount of a 5-HT2A receptor antagonist. In some aspects, the method can further comprise administering to the subject a therapeutically effective amount of one or more of the decoy peptides, polypeptides or fragments thereof disclosed herein. In some aspects, the sample can be plasma or serum. In some aspects, the level of binding of 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the level of binding of 5-HT2A receptor autoantibodies to the one or more decoy peptides comprising or consisting of the sequence of QDDSKVFKEGSCLLADDN (SEQ ID NO: 1) or a fragment thereof can be determined by ELISA. In some aspects, the 5-HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907).

Disclosed herein are methods of identifying a subject at risk for elevated levels of alanine aminotransferase (ALT). In some aspects, the subject has type 2 diabetes, essential hypertension, or obesity. In some aspects, the subject does not have or has not been diagnosed with type 2 diabetes, essential hypertension or obesity. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); and d) identifying the subject at risk for elevated levels of ALT when the level of binding in step c) is higher when compared to the level of binding in a reference sample. In some aspects, the methods can comprise: a) obtaining or having obtained a sample from the subject, wherein the sample can comprise one or more 5-HT2A receptor autoantibodies; b) contacting the sample of step a) with one or more decoy peptides comprising or consisting of the sequence of QDDSKVFKEGSCLLADDN (SEQ ID NO: 1) or a fragment thereof; c) determining the level of binding of the 5-HT2A receptor autoantibodies to the one or more decoy peptides comprising or consisting of the sequence of QDDSKVFKEGSCLLADDN (SEQ ID NO: 1) or a fragment thereof; and d) identifying the subject at risk for developing hypertriglyceridemia when the level of binding in step c) is higher when compared to the level of binding in a reference sample. In some aspects, the method can further comprise administering to the subject a therapeutically effective amount of a 5-HT2A receptor antagonist. In some aspects, the methods can comprise: i) obtaining or having obtained a sample from the subject, wherein the sample can comprise one or more 5-HT2A receptor autoantibodies; ii) contacting the sample of step i) with one or more decoy peptides comprising or consisting of the sequence of QDDSKVFKEGSCLLADDN (SEQ ID NO: 1) or a fragment thereof; iii) determining the level of binding of the 5-HT2A receptor autoantibodies to the one or more decoy peptides comprising or consisting of the sequence of QDDSKVFKEGSCLLADDN (SEQ ID NO: 1) or a fragment thereof; and iv) identifying the subject at risk for developing hypertriglyceridemia when the level of binding in step iii) is higher when compared to the level of binding in a reference sample. In some aspects, the method can further comprise administering to the subject a therapeutically effective amount of one or more of the decoy peptides, polypeptides or fragments thereof disclosed herein. In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated. In some aspects, the sample can be plasma or serum. In some aspects, the level of binding of 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the level of binding of 5-HT2A receptor autoantibodies to the one or more decoy peptides comprising or consisting of the sequence of QDDSKVFKEGSCLLADDN (SEQ ID NO: 1) or a fragment thereof can be determined by ELISA. In some aspects, the 5-HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907).

Disclosed herein are methods of treating a subject having or at risk for developing hypertriglyceridemia in a subject. In some aspects, the subject has type 2 diabetes, is obese or a combination thereof. In some aspects, the methods can comprise: a) administering to the subject a therapeutically effective amount of a 5-HT2A receptor antagonist. In some aspects, the subject has been identified to have or be at risk of developing hypertriglyceridemia by a method comprising: i) obtaining or having obtained a sample from the subject, wherein the sample can comprise one or more 5-HT2A receptor autoantibodies; ii) contacting the sample of step i) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); iii) determining the level of binding of the receptor autoantibodies to the one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); iv) identifying the subject having or at risk for developing hypertriglyceridemia when the level of binding in step iii) is higher when compared to the level of binding in a reference sample. In some aspects, step a) can comprise administering to the subject a therapeutically effective amount of a decoy peptide or polypeptide disclosed herein. In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated. In some aspects, the sample can be plasma or serum. In some aspects, the level of binding of 5-HT2A receptor autoantibodies to one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the -HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907).

Disclosed herein are methods treating hypertriglyceridemia in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating obesity in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating diabetic dyslipidemia in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating premature atherosclerosis in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating atherosclerotic heart disease in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating fatty liver disease in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can do myristolated.

Disclosed herein are methods treating a subject at risk for having a stroke or preventing or ameliorating a symptom of stroke. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated. In some aspects, the subject has or has been diagnosed with hypertension. Symptoms of stroke include but are not limited to headache, weakness, dysarthria, confusion, aphasia, loss of sensation, ataxia, and sudden loss of vision. Risk factors for stroke include but are not limited to hypertension, diabetes, morbid obesity, hypertriglyceridemia, hyperlipidemia, and serotonin syndrome due to excess circulating serotonin. In some aspects the stroke can be ischemic stroke, transient ischemia attack, hemorrhagic stroke, retinal vein occlusion, retinal artery occlusion, cerebral vasospasm, or serotonin syndrome-related stroke.

Disclosed herein are methods treating hypertension in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods treating a subject at risk for developing liver cirrhosis or liver failure. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated. In some aspects, the subject has or has been diagnosed with fatty liver disease. Symptoms of fatty liver disease include but are not limited to loss of appetite, skin discoloration, fatigue, difficulty concentrating, abdominal swelling, change in bowel movements, change in urine, bruising or bleeding, swelling in legs, and increased blood pressure. Risk factors for fatty liver disease, liver cirrhosis or liver failure include but are not limited to alcohol overuse, smoking, overuse of pain medications, hypertriglyceridemia, abdominal obesity, and certain genetic diseases including but not limited to Wilson's disease, infectious hepatitis, and autoimmune diseases.

Disclosed herein are methods preventing cardiac steatosis in a subject. In some aspects, the methods can comprise: administering to a subject a therapeutically effective amount of a decoy peptide disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of reducing or preventing cardiac hypertrophy in a subject. In some aspects, the methods can comprise: administering to a subject a therapeutically effective amount of one or more decoy peptides, polypeptides or fragments thereof disclosed herein. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of reducing or preventing acute neurite retraction in a subject. In some aspects, the methods can comprise: administering to a subject a therapeutically effective amount of one or more decoy peptides, polypeptides or fragments thereof disclosed herein. In some aspects, the methods can further comprise: administering a therapeutically effective amount of one or more decoy peptides, polypeptides or fragments thereof disclosed herein and a pharmaceutically acceptable carrier. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of preventing accelerated neuron loss in a subject. In some aspects, the methods can comprise: administering to a subject a therapeutically effective amount of one or more decoy peptides, polypeptides or fragments thereof disclosed herein. In some aspects, the methods can further comprise: administering a therapeutically effective amount of one or more decoy peptides, polypeptides or fragments thereof disclosed herein and a pharmaceutically acceptable carrier. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of reducing the risk of a neurologic disease or disorder or microvascular disease or disorder in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of preventing or reducing cognitive decline (cognitive dysfunction including changes in memory), mood or one or symptoms associated with depression in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, the subject has type 2 diabetes. In some aspects, the subject has type 2 diabetes and a traumatic injury. In some aspects, the subject was diagnosed with type 2 diabetes before the traumatic brain injury. In some aspects, the subject has a traumatic brain injury. In some aspects, the subject does not have or has not been diagnosed with type 2 diabetes. In some aspects, the subject does not have or has not been diagnosed with a traumatic brain injury. In some aspects, the subject has neither type 2 diabetes nor traumatic brain injury or has been diagnosed with type 2 diabetes or a traumatic brain injury. In some aspects, the subject has essential hypertension or obesity. In some aspects, the methods can comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the methods can further comprise: administering a therapeutically effective amount of one or more decoy peptides, polypeptides or fragments thereof disclosed herein and a pharmaceutically acceptable carrier. In some aspects, the decoy peptide can comprise or consist of the sequence SCLLADDN (SEQ ID NO: 2), QDDSKVF (SEQ ID NO: 3), or VFKEGSC (SEQ ID NO: 4). In some aspects, the decoy peptide can comprise or consist of SCLLADDN (SEQ ID NO: 2). In some aspects, the decoy peptide can further comprise at least one polyethylene glycolated (PEGylated) group. In some aspects, the decoy peptide can further comprise at least one polyethylene glycolated (PEGylated) group attached to the N-terminus. In some aspects, the decoy peptide further comprises a fatty acid. In some aspects, the decoy peptide can be myristolated.

Disclosed herein are methods of inducing sedation in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated.

Disclosed herein are methods of reducing weight gain in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated. In some aspects, the subject is diabetic and obese. In some aspects, the subject does not have type 2 diabetes. In some aspects, the subject does not have or has not been diagnosed with type 2 diabetes. In some aspects, the subject is obese. In some aspects, reducing weight gain can be independent of blood glucose concentrations.

Disclosed herein are methods of promoting or inducing weight loss in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated. In some aspects, the subject is diabetic and obese. In some aspects, the subject is not diabetic. In some aspects, the subject is obese. In some aspects, weight loss is not related to and is independent from changes in blood glucose concentrations.

Disclosed herein are methods of improving short term memory in a subject. Disclosed herein are methods of improving short term recall in a subject. Disclosed herein are methods of improving spatial memory in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated. In some aspects, the subject is diabetic and obese. In some aspects, the subject has type 2 diabetes. In some aspects, the subject is obese. In some aspects, the subject has type 2 diabetes and can be obese. In some aspects, the subject has neither type 2 diabetes nor traumatic brain injury or has been diagnosed with type 2 diabetes or a traumatic brain injury. In some aspects, the subject has neither type 2 diabetes nor is obese or has been diagnosed with type 2 diabetes or obesity.

Disclosed herein are methods of ameliorating a symptom of dementia, Alzheimer's disease or a neurodegenerative disease in a subject. In some aspects, the symptom can be short term memory loss. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated. In some aspects, the subject is diabetic and obese. In some aspects, the subject has type 2 diabetes. In some aspects, the subject is obese. In some aspects, the subject has type 2 diabetes and can be obese. In some aspects, the subject has neither type 2 diabetes nor traumatic brain injury or has been diagnosed with type 2 diabetes or a traumatic brain injury. In some aspects, the subject has neither type 2 diabetes nor is obese or has been diagnosed with type 2 diabetes or obesity.

Disclosed herein are methods of preventing or reducing one or more symptoms of retinal degeneration or the risk of developing retinal degeneration in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated.

Disclosed herein are methods of lowering blood pressure or lowering hypertension in a subject. Also disclosed herein are methods of lowering or decreasing systolic or diastolic blood pressure in a subject. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated. In some aspects, the subject is diabetic and obese. In some aspects, the subject has type 2 diabetes. In some aspects, the subject is obese. In some aspects, the subject is not obese. In some aspects, the subject does not have or has not been diagnosed with type 2 diabetes. In some aspects, the subject can be have mild or high blood pressure. In some aspects, the subject can be have mild or high blood pressure and has not been diagnosed with any known disease, disorder or condition. In some aspects, the methods can further reduce or prevent cardiac hypertrophy in subjects with hypertension or high blood pressure. In some aspects, the high blood pressure can be considered moderate to severe.

Blood pressure is the force exerted by the bloodstream on the artery walls. Normal blood pressure is considered to be 120 systolic and 80 diastolic. Hypertension refers to a disorder that can be characterized by an elevation of the systolic blood pressure to 140 and above and/or an elevation of the diastolic blood pressure to 90 and above. With hypertension, there is either an increase in the total peripheral vascular resistance such as is due to vasoconstriction, or an increase in cardiac output, or both. These conditions produce an elevation in blood pressure because blood pressure is equal to flow times resistance. Many factors can contribute to high blood pressure including but not limited to stress, diet and lifestyle, as well as kidney complaints, hormonal disturbances and circulatory disorders. An untreated hypertensive patient is at great risk of developing left ventricular failure, myocardial infarction, cerebral hemorrhage or renal failure. Hypertension can also be a risk factor for stroke and coronary atherosclerosis. Currently, hypertensive patients are treated with drug therapy that includes the use of diuretics, beta-blockers, ACE inhibitors, angiotensin antagonists, calcium channel blockers, alpha-blockers, alpha-beta-blockers, nervous system inhibitors, and vasodilators. Essential hypertension is high blood pressure that doesn't have a known secondary cause. Refractory hypertension is defined as uncontrolled blood pressure despite use of five or more antihypertensive agents of different classes. In some aspects, mild hypertension can be defined as from 140/90 mm Hg. In some aspects, moderate hypertension can be defined as from 160/100 mm Hg. In some aspects, severe hypertension can be defined as from 180/110 mm Hg.

Disclosed herein are methods of preventing or reducing one or more symptoms of kidney disease or the risk of developing kidney disease in a subject. In some aspects, the kidney disease can be diabetic kidney disease. In some aspects, the methods can comprise: administering to a subject a therapeutically effective amount of one or more of the decoy peptides, polypeptides or fragments thereof described herein. In some aspects, the methods can further comprise: administering a therapeutically effective amount of one or more decoy peptides, polypeptides or fragments thereof disclosed herein and a pharmaceutically acceptable carrier. In some aspects, a therapeutically effective amount of SCLLADDN (SEQ ID NO: 2) or a composition comprising or consisting of SCLLADDN (SEQ ID NO: 2) can be administered to the subject. In some aspects, a therapeutically effective amount of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof or a composition comprising or consisting of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof can be administered to the subject. In some aspects, the decoy peptide further comprises a fatty acid. In some aspects, the decoy peptide can be myristolated.

Also disclosed herein are methods of identifying a subject at risk for developing a neurodegenerative disease. In some aspects, the subject has type 2 diabetes, hypertension, or obesity. In some aspects, the subject does not have type 2 diabetes or has not been diagnosed with type 2 diabetes. In some aspects, the subject does not have hypertension or has not been diagnosed with hypertension. In some aspects, the subject is not obese. In some aspects, the subject has a traumatic brain injury. In some aspects, the subject has a traumatic brain injury and type 2 diabetes. In some aspects, the subject has a traumatic brain injury, type 2 diabetes and obesity. In some aspects, subject does not have or has been diagnosed with any of traumatic brain injury, type 2 diabetes or obesity. In some aspects, the method comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); and d) identifying the subject at risk for developing a neurodegenerative disease when the level of binding in step c) is higher when compared to the level of binding in a reference sample. In some aspects, the method can further comprise administering to the subject a therapeutically effective amount of a 5-HT2A receptor antagonist. In some aspects, the method can further comprise administering to the subject a therapeutically effective amount of one or more of the decoy peptides, polypeptides or fragments thereof disclosed herein. In some aspects, the sample can be plasma or serum. In some aspects, the level of binding of 5-HT2A receptor autoantibodies to one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the 5-HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907).

Disclosed herein are methods of treating a subject having or at risk for developing a neurodegenerative disease or preventing or ameliorating a symptom of neurodegenerative disease in a subject. In some aspects, the subject has type 2 diabetes, refractory hypertension, essential hypertension, or obesity. In some aspects, the subject does not have or has not been diagnosed with type 2 diabetes, refractory hypertension, essential hypertension or obesity. In some aspects, the subject has a traumatic brain injury. In some aspects, the subject has a traumatic brain injury and type 2 diabetes. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); d) identifying the subject having or at risk for developing a neurodegenerative disease when the level of binding in step c) is higher when compared to the level of binding in a reference sample; and e) administering to the subject a therapeutically effective amount of a 5-HT2A receptor antagonist. In some aspects, step e) can comprise administering to the subject a therapeutically effective amount of a decoy peptide or polypeptide disclosed herein. In some aspects, the sample can be plasma or serum. In some aspects, the level of binding of 5-HT2A receptor autoantibodies to one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the -HT2A receptor antagonist can inhibit the binding of 5-HT2A receptor autoantibodies to a second extracellular loop region of the 5-HT2A receptor. In some aspects, the 5-HT2A receptor antagonist can inhibit the binding of receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907). In some aspects, the decoy peptide further comprises a fatty acid. In some aspects, the decoy peptide can be myristolated.

Also disclosed herein are methods of identifying a subject at risk for or developing retinal degeneration. In some aspects, the subject has type 2 diabetes, essential hypertension, or obesity. In some aspects, the subject does not have or has not been diagnosed with type 2 diabetes, essential hypertension or obesity. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); and d) identifying the subject at risk for developing a retinal degeneration when the level of binding in step c) is higher when compared to the level of binding in a reference sample. In some aspects, the method can further comprise administering to the subject a therapeutically effective amount of a 5-HT2A receptor antagonist. In some aspects, the method can further comprise administering to the subject a therapeutically effective amount of one or more of the decoy peptides, polypeptides or fragments thereof disclosed herein. In some aspects, the sample can be plasma or serum. In some aspects, the level of binding of 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the 5-HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907). In some aspects, the decoy peptide further comprises a fatty acid. In some aspects, the decoy peptide can be myristolated.

Disclosed herein are methods of treating a subject having or at risk for developing retinal degeneration. In some aspects, the subject has type 2 diabetes. In some aspects, the subject has type 2 diabetes, essential hypertension, or obesity. In some aspects, the subject does not have or has not been diagnosed with type 2 diabetes, essential hypertension or obesity. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); d) identifying the subject at risk for developing a retinal degeneration when the level of binding in step c) is higher when compared to the level of binding in a reference sample; and e) administering to the subject a therapeutically effective amount of a 5-HT2A receptor antagonist. In some aspects, step e) can comprise administering to the subject a therapeutically effective amount of a decoy peptide or polypeptide disclosed herein. In some aspects, the sample can be plasma or serum. In some aspects, the level of binding of 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the -HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907). In some aspects, the decoy peptide further comprises a fatty acid. In some aspects, the decoy peptide can be myristolated.

Disclosed herein are methods of treating a subject having or at risk for kidney disease, kidney dysfunction or kidney failure. In some aspects, the subject has type 2 diabetes or refractory hypertension. In some aspects, the methods can comprise: a) obtaining or having obtained a sample from the subject, wherein the sample can comprise one or more 5-HT2A receptor autoantibodies; b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); c) determining the level of binding of the 5-HT2A receptor autoantibodies to the one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); d) identifying the subject at risk for kidney disease, kidney dysfunction or kidney failure when the level of binding in step c) is higher when compared to the level of binding in a reference sample; and e) administering to the subject a therapeutically effective amount of a 5-HT2A receptor antagonist. In some aspects, step e) can comprise administering to the subject a therapeutically effective amount of a decoy peptide or polypeptide disclosed herein. In some aspects, the sample can be plasma or serum. In some aspects, the level of binding of 5-HT2A receptor autoantibodies to the one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) can be determined by ELISA. In some aspects, the -HT2A receptor antagonist can inhibit the binding of 5-HT2A receptor autoantibodies to a second extracellular loop region of the 5-HT2A receptor. In some aspects, the 5-HT2A receptor antagonist can inhibit the binding of 5-HT2A receptor autoantibodies to a second extracellular loop region of the 5-HT2A receptor by competitive inhibition. In some aspects, the 5-HT2A receptor antagonist can be a decoy peptide or polypeptide. In some aspects, the decoy peptide can inhibit the binding of 5-HT2A receptor autoantibodies to a second extracellular loop region of the 5-HT2A receptor. In some aspects, the decoy peptide can inhibit the binding of 5-HT2A receptor autoantibodies to a second extracellular loop region of the 5-HT2A receptor by competitive inhibition. In some aspects, the subject can be identified in need of treatment before the administration step. In some aspects, the subject can be human. In some aspects, the method can further comprise administering to the subject ketanserin or volinanserin (also known as MDL-100,907). In some aspects, the decoy peptide further comprises a fatty acid. In some aspects, the decoy peptide can be myristolated.

Disclosed herein are methods of treating a subject having or at risk for developing obesity or preventing or ameliorating a symptom of obesity in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject having or at risk for developing a type 2 diabetes or preventing or ameliorating a symptom of type 2 diabetes in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject having or at risk for developing a premature atherosclerosis or preventing or ameliorating a symptom of premature atherosclerosis in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject having or at risk for developing atherosclerotic heart disease or preventing or ameliorating a symptom of atherosclerotic heart disease in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject at risk for having a stroke or preventing or ameliorating a symptom of stroke in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject having or at risk for developing fatty liver disease or preventing or ameliorating a symptom of fatty liver disease in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

Disclosed herein are methods of treating a subject having or at risk for developing cardiac hypertropy or preventing or ameliorating a symptom of hypertension in a subject. In some aspects, the methods comprise: administering to the subject a therapeutically effective amount of a decoy peptide, wherein the decoy peptide comprises or consists of the sequence QDDSKVFKEGSCLLADDN (SEQ ID NO: 1), or a fragment thereof. In some aspects, the decoy peptide further comprises a fatty acid. In some aspects, the decoy peptide can be myristolated.

In some aspects of the methods disclosed here, any of the decoy peptides described herein can further comprise at least one polyethylene glycolated (PEGylated) group attached to the N-terminus. In some aspects of the methods disclosed herein, the decoy peptides, polypeptides or fragments thereof described herein can be part of a scaffold protein. In any of the methods disclosed herein, the scaffold protein can comprise a SH3 (src homolog3), SH2 (src homolog2), PDZ, or GTPase-binding domain (GBD) and a heterologous peptide. In some aspects, the heterologous peptide can be inserted or connected to one or more of a SH3 (src homolog3), SH2 (src homolog2), PDZ, or GTPase-binding domain (GBD). In some aspects, the heterologous peptide can be one or more of the decoy peptides, polypeptides or fragments thereof disclosed herein. In some aspects, the heterologous peptide can comprise or consist of one or more decoy peptides comprising the sequence of QDDSKVFKEGSCLLADDN (SEQ ID NO: 1), or a fragment thereof, SCLLADDN (SEQ ID NO: 2), QDDSKVF (SEQ ID NO: 3), or VFKEGSC (SEQ ID NO: 4). In some aspects, the heterologous peptide can comprise or consist of one or more decoy peptides comprising a variant of the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4). In some aspects, the method can comprise administering a composition that can be formulated for intravenous, subcutaneous, intranasal or oral administration. In some aspects, the decoy peptide further comprises a fatty acid. In some aspects, the decoy peptide can be myristolated.

In some aspects, the 5-HT2A receptor antagonist can be any of the decoy peptides, polypeptides or fragments thereof disclosed herein. In some aspects, the 5-HT2A receptor antagonist can be SCLLADDN (SEQ ID NO: 2). In some aspects, the 5-HT2A receptor antagonist can be QDDSKVFKEGSCLLADDN (SEQ ID NO: 1) or a fragment thereof. In some aspects, the 5-HT2A receptor antagonist can be QDDSKVF (SEQ ID NO: 3). In some aspects, the 5-HT2A receptor antagonist can be VFKEGSC (SEQ ID NO: 4).

In some aspects, the level of binding of the 5-HT2A receptor autoantibodies in the sample can be increased or higher when compared to the level of binding of 5-HT2A receptor autoantibodies in a control or reference sample. In some aspects, a sample from a subject can be identified as being in need of treatment when the level of binding of the 5-HT2A receptor autoantibodies in the sample can be increased or higher when compared to the level of binding of 5-HT2A receptor autoantibodies in a control or reference sample. In some aspects, the sample from the subject can be identified as being at risk for developing a neurodegenerative disease, retinal degeneration, or kidney disease, kidney dysfunction or kidney failure. In some aspects, the control or reference sample can be from an age-matched sample. In some aspects, the sample can be from one or more subjects that do have or are known to not be at risk for developing a neurodegenerative disease or retinal degeneration.

As used herein, the terms, “reference,” “reference expression,” “reference sample,” “reference value,” “control,” “control sample” and the like, when used in the context of a sample or expression level of the binding of 5HT2A receptor autoantibodies to one or more of the decoy peptides disclosed herein refers to a reference standard wherein the reference is expressed at a constant level, and is unaffected by the experimental conditions, and is indicative of the level in a sample of a predetermined disease status (e.g., not suffering from a neurodegenerative disease, type 2 diabetes, retinal degeneration) or whether a subject (or disease) will respond to a therapeutic agent or treatment. The reference value can be a predetermined standard value or a range of predetermined standard values, representing no illness, or a predetermined type or severity of illness or representing the likelihood a disease, disorder or condition will be responsive to a particular type of therapeutic agent or treatment.

Reference expression can be the level of the binding of 5HT2A receptor autoantibodies to one or more of the decoy peptides disclosed herein in a reference sample from a subject, or a pool of subjects, not suffering from disease, disorder or condition (e.g., a neurodegenerative disease, refractory hypertension, retinal degeneration), with a known response (or lack thereof) to a particular treatment or known to be at risk for having or developing a disease, disorder or condition (e.g., a neurodegenerative disease, refractory hypertension, retinal degeneration). In some aspects, the reference value can be taken a different time point than to which it is being compared.

As used herein, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual before administration of or exposure to a particular therapeutic agent, but at an earlier point in time, or a value obtained from a sample from a subject other than the individual being tested, or a “normal” individual, that is an individual not diagnosed with any of the diseases, disorders or conditions described herein. The reference value can be based on a large number of samples, such as from subjects with any of the diseases, disorders or conditions described herein or normal individuals or based on a pool of samples including or excluding the sample to be tested. The reference value can also be based on a sample from subjects with any of the diseases, disorders or conditions described herein other than the individual being tested, or a “normal” individual that is an individual not diagnosed with any of the diseases, disorders or conditions described herein that has not or has been administered or exposed to a particular therapeutic agent.

The reference level used for comparison with the measured level for the binding of receptor autoantibodies to any of the decoy peptides disclosed herein can vary, depending the method begin practiced, as will be understood by one of ordinary skill in the art. For methods for determining the likelihood a disease, disorder or condition (e.g., a neurodegenerative disease, type 2 diabetes, retinal degeneration), a subject or a sample will be responsive to a particular type of therapeutic agent or treatment, the “reference level” is typically a predetermined reference level, such as an average of levels obtained from a population that has either been exposed or has not been exposed to particular type of therapeutic agent or treatment, but in some instances, the reference level can be a mean or median level from a group of individuals that are responders or non-responders. In some instances, the predetermined reference level can be derived from (e.g., is the mean or median of) levels obtained from an age-matched population.

Age-matched populations (from which reference values may be obtained) can be populations that are the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-year old individuals, or 62-66 year old individuals).

Determining the level of binding of the 5-HT2A receptor autoantibodies to one or more of the decoy peptides disclosed herein can include determining whether the binding of the 5-HT2A receptor autoantibodies to one or more of the decoy peptides disclosed herein is increased as compared to a control or reference sample or a sample that has been contacted, administered or exposed to a particular therapeutic agent or treatment, decreased compared to a control or reference sample or a sample that has been contacted, administered or exposed to a particular therapeutic agent or treatment, or unchanged compared to a control or reference sample or a sample that has been contacted, administered or exposed to a particular therapeutic agent or treatment. As used herein, the terms, “increased” or “increased expression level” or “increased level of expression” or “increased amount of protein” or “high” or “higher level” or “higher expression level” refers to an amount of binding of the 5-HT2A receptor autoantibodies to one or more of the decoy peptides disclosed herein that is expressed wherein the measure of the quantity of the binding of the 5-HT2A receptor autoantibodies to one or more of the decoy peptides disclosed herein exhibits an increased level of expression when compared to a reference sample or “normal” control or a sample that has been contacted, administered or exposed to a particular therapeutic agent or treatment. An “increased expression level” or “higher expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “decreased,” “decreased level of expression,” or “decreased expression level” or “decreased amount of protein” or “low” or “lower level” or “lower expression level” refers to an amount of binding of the 5-HT2A receptor autoantibodies to one or more of the decoy peptides disclosed herein that is expressed wherein the measure of the quantity of the binding of the 5-HT2A receptor autoantibodies to one or more of the decoy peptides disclosed herein exhibits a decreased level of expression when compared to a reference sample or “normal” control or a sample that has been contacted, administered or exposed to a particular therapeutic agent or treatment. A “decreased level of expression” or “lower expression level” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

The level of binding of the 5-HT2A receptor autoantibodies to one or more of the decoy peptides disclosed herein disclosed herein can be a measure, for example, per unit weight or volume. In some aspects, the expression level can be a ratio (e.g., the amount of binding of the 5-HT2A receptor autoantibodies to one or more of the decoy peptides disclosed herein in a sample relative to the amount of the binding of the 5-HT2A receptor autoantibodies to one or more of the decoy peptides disclosed herein of a reference value or in a reference sample that may have been or may have not been also contacted with a therapeutic agent).

The method of comparing a measured value and a reference value or a measured value before and after contact with a therapeutic agent can be carried out in any convenient manner appropriate to the type of measured value (e.g., the binding of the 5-HT2A receptor autoantibodies to one or more of the decoy peptides disclosed herein). For example, ‘measuring’ can be performed using quantitative or qualitative measurement techniques, and the mode of comparing a measured value and a reference value can vary depending on the measurement technology employed. For example, the measured values used in the methods described herein can be quantitative values (e.g., quantitative measurements of concentration, such as nanograms of the 5HT2A receptor autoantibodies per milliliter of sample, or absolute amount). As with qualitative measurements, the comparison can be made by inspecting the numerical data, by inspecting representations of the data (e.g., inspecting graphical representations such as bar or line graphs).

In some aspects, the level of binding of 5-HT2A receptor autoantibodies can be determined by various analysis methods. For example, the binding of 5-HT2A receptor autoantibodies can be determined in various immunoassay formats. These immunological analysis methods may be carried out according to various quantitative immunoassay protocols that have been developed in the prior art. Examples of the immunoassay format include radioactive immunoassay, radioactive immunoprecipitation, immunoprecipitation, enzyme-linked immunosorbent assay (ELISA), captured-ELISA, inhibition or competition analysis, sandwich assay, immunofluorescent staining, and immunoaffinity purification, but are not limited thereto.

In some aspects, in any of the methods disclosed herein, the level of binding of 5-HT2A receptor antibodies can be determined in a sample prior the administration of a decoy peptide or a composition comprising a decoy peptide to determine whether the subject will respond to the administration of a decoy peptide or a composition comprising a decoy peptide. In some aspects, the method can comprise obtaining or having obtained a sample from the subject, wherein the sample comprises one or more 5-HT2A receptor autoantibodies, contacting the sample with one or more decoy peptides comprising the sequence of QDDSKVFKEGSCLLADDN (SEQ ID NO: 1), SCLLADDN (SEQ ID NO: 2), QDDSKVF (SEQ ID NO: 3), OR VFKEGSC (SEQ ID NO; 4), determining the level of binding of the 5HT2A receptor autoantibodies to one or more of the decoy peptides comprising QDDSKVFKEGSCLLADDN (SEQ ID NO: 1), SCLLADDN (SEQ ID NO: 2), QDDSKVF (SEQ ID NO: 3), OR VFKEGSC (SEQ ID NO; 4), thereby determining whether the subject will respond to administration of a decoy peptide or a composition comprising a decoy peptide. When said binding is detected, it is indicated that the subject will respond to administration of a decoy peptide or a composition comprising a decoy peptide. When no binding is detected, it is indicated that the subject will not respond to administration of a decoy peptide or a composition comprising a decoy peptide. In some aspects, the decoy peptide further comprises a fatty acid. In some aspects, the decoy peptide can be myristolated.

Disclosed herein are methods of competitively inhibiting the binding of 5-HT2A receptor autoantibodies to a second extracellular loop region of the 5-HT2A receptor. In some aspects, the method can comprise: administering to a subject a therapeutically effective amount of a peptide comprising or consisting of the amino acid sequence of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1) or a fragment thereof. In some aspects, the method can comprise: administering to a subject a therapeutically effective amount of a peptide comprising or consisting of a variant of the amino acid sequence of SCLLADDN (SEQ ID NO: 2), QDDSKVF (SEQ ID NO: 3), VFKEGSC (SEQ ID NO: 4) or QDDSKVFKEGSCLLADDN (SEQ ID NO: 1). In some aspects, the decoy peptide further comprises a fatty acid. In some aspects, the decoy peptide can be myristolated. In some aspects, the subject can be identified in need of treatment before the administration step. In some aspects, the subject can be human. In some aspects, the method can further comprise administering to the subject ketanserin or volinanserin (also known as MDL-100,907). In some aspects, the subject can be at risk for developing hypertriglyceridemia. In some aspects, the subject can be at risk for having a stroke. In some aspects, the subject can be at risk for developing liver cirrhosis or liver failure. In some aspects, the subject has type 2 diabetes, a neurologic disease or disorder, a microvascular disease or disorder, hypertension, schizophrenia, retinitis pigmentosa, mild cognitive dysfunction, primary open angle glaucoma, diabetic dyslipidemia, fatty liver disease, type 2 hyperlipidemia, hypertriglyceridemia, microvascular angiopathy, diabetic kidney disease, Parkinson's disease, dementia, major depressive disorder, obesity, refractory hypertension, essential hypertension or has had a stroke or a traumatic brain injury, is overweight, or a combination thereof. In some aspects, the subject can be at risk for developing retinal degeneration, kidney disease, a neurologic disease or disorder, kidney dysfunction, kidney failure, a microvascular disease or disorder, or a neurodegenerative disease or disorder. In some aspects, the neurologic disease or disorder can be a neuropathy, open angle glaucoma, dementia, major depressive disorder, Parkinson's disease, dementia or a combination thereof. In some aspects, the microvascular disease or disorder can be a stroke or kidney failure. In some aspects, the subject has type 2 diabetes. In some aspects, the subject has a traumatic brain injury. In some aspects, the subject has type 2 diabetes, hypertension or obesity or has been diagnosed with type 2 diabetes or hypertension. In some aspects, the subject does not have or has not been diagnosed with type 2 diabetes, hypertension or obesity. In some aspects, the subject has type 2 diabetes and a traumatic brain injury. In some aspects, the subject has obesity. In some aspects, the subject has refractory hypertension or essential hypertension. In some aspects, the subject has moderate to severe hypertension.

Disclosed herein are methods of competitively inhibiting the binding of 5-HT2A receptor autoantibodies to a second extracellular loop region of the 5-HT2A receptor. In some aspects, the method can comprise: administering to a subject a therapeutically effective amount of any of decoy peptide or polypeptides disclosed herein. In some aspects, the methods can comprise: administering to a subject a therapeutically effective amount of any of the compositions disclosed herein. In some aspects, the subject can be identified in need of treatment before the administration step. In some aspects, the subject can be human. In some aspects, the method can further comprise administering to the subject ketanserin or volinanserin (also known as MDL-100,907). In some aspects, the subject can be at risk for developing retinal degeneration, kidney disease, kidney dysfunction, kidney failure, a neurologic disease or disorder, or a microvascular disease or disorder, or a neurodegenerative disease or disorder. In some aspects, the subject can be at risk for developing kidney dysfunction or kidney failure. In some aspects, the subject can be at risk for developing hypertriglyceridemia. In some aspects, the subject can be at risk for having a stroke. In some aspects, the subject can be at risk for developing liver cirrhosis or liver failure. In some aspects, the subject can be at risk for cardiac steatosis. In some aspects, the neurologic disease or disorder can be a neuropathy, open angle glaucoma, dementia, major depressive disorder, Parkinson's disease or a combination thereof. In some aspects, the microvascular disease or disorder can be a stroke or kidney dysfunction or kidney failure. In some aspects, the subject has type 2 diabetes. In some aspects, the subject has or has been diagnosed with type 2 diabetes, essential hypertension, or obesity. In some aspects, the subject has or has been diagnosed with a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia, is overweight or has had a stroke or a traumatic brain injury or a combination thereof. In some aspects, the subject does not have or has not been diagnosed with type 2 diabetes, essential hypertension or obesity. In some aspects, the subject has a traumatic brain injury. In some aspects, the subject has type 2 diabetes and a traumatic brain injury. In some aspects, the subject has obesity. In some aspects, the subject has refractory hypertension or essential hypertension. In some aspects, the subject has moderate to severe hypertension.

In some aspects, the subject can be identified as being in need of treatment before the administration step. In some aspects, the subject can have type 2 diabetes, a traumatic brain injury, microvascular angiopathy (including diabetic or hypertensive nephropathy), a neurologic disease or disorder, a microvascular disease or disorder, hypertension, Parkinson's disease, dementia, major depressive disorder, schizophrenia, retinitis pigmentosa, refractory hypertension, essential hypertension, hypertension, mild cognitive dysfunction, primary open angle glaucoma, diabetic dyslipidemia, hypertriglyceridemia, type 2B hyperlipidemia, obesity or has had a stroke, is overweight or a combination thereof. In some aspects, a subject can be determined to be overweight by calculating the subject's body mass index (BMI). BMI is a value defined as the body mass of a subject dived by the square of the body height, and is expressed in units of kg/m². In some aspects, a BMI of about 25 to about 29.9 is considered overweight. In some aspects, a BMI of about 30 or higher is considered obese. Hypertriglyceridemia means high blood (or plasma) levels of triglycerides. Triglycerides are the most abundant fatty molecule in most organisms. Elevated levels of triglycerides can be elevated in the absence of hypercholesterolemia (high cholesterol levels). In some aspects, the subject can be identified as being as at risk for hypertriglyceridemia or elevated ALT levels by determining the subject's level of triglycerides or ALT levels, respectively, before the administration step. In some aspects, hypertriglyceridemia can be defined by the amount of blood or plasma levels of triglycerides, for example, an amount of 150 mg/dL (1.7 mmol/L) or higher. In some aspects, the subject is not obese. In some aspects, the subject does not have type 2 diabetes. In some aspects, the subject does not have a traumatic brain injury.

In some aspects, the neurologic disease or disorder can be a neuropathy, open angle glaucoma, dementia, major depressive disorder, Parkinson's disease or a combination thereof.

In some aspects, the microvascular disease or disorder can be a stroke. In some aspects, the microvascular disease or disorder can be age-related macular degeneration, diabetic macular edema, diabetic nephropathy, hypertensive nephropathy.

In some aspects, the refractory hypertension can be associated with stroke or chronic kidney disease. In some aspects, the neurodegenerative disease can be dementia, Parkinson's disease, macular degeneration, or retinal degeneration.

In some aspects, in any of the methods disclosed herein, the methods can further comprise: administering a therapeutically effective amount of one or more decoy peptides, polypeptides or fragments thereof disclosed herein. In some aspects, any of the decoy peptides can further comprise at least one polyethylene glycocated (PEGylated) group. In some aspects, any of the decoy peptides can further comprise at least one polyethylene glycocated (PEGylated) group attached to the N-terminus. In some aspects, the one or more decoy peptides, polypeptides or fragments thereof disclosed herein can be administered along with a pharmaceutically acceptable carrier.

In some aspects, in any of the methods disclosed herein, the plasma levels of aspartate aminotransferase (AST) in the subject decreases as compared to plasma AST levels prior to administering the therapeutically effective amount of the decoy peptide. In some aspects, in any of the methods disclosed herein, the plasma levels of aspartate aminotransferase (AST) in the subject remain unchanged or do not increase as compared to plasma AST levels prior to administering the therapeutically effective amount of the decoy peptide.

In some aspects, in any of the methods disclosed herein, the plasma levels of aspartate aminotransferase (AST) or ALT in the subject decreases as compared to plasma AST levels prior to administering the therapeutically effective amount of the decoy peptide. In some aspects, in any of the methods disclosed herein, the plasma levels of aspartate aminotransferase (AST) in the subject remain unchanged or do not increase as compared to plasma AST levels prior to administering the therapeutically effective amount of the decoy peptide.

In some aspects, in any of the methods disclosed herein, the subject's body weight change is lowered (or less) as compared to the subject's body weight change without administration of the therapeutically effective amount of the decoy peptide.

In some aspects, any of the decoy peptides, polypeptides or fragments thereof disclosed herein can be delivered or administered to a subject by direct injection into the eye, for example, to treat retinal degenerative disorder; by direct or local injection into the brain or cerebrospinal fluid, for example, to treat brain a neurodegenerative disorder; and by direct injection into an occluded arterial region via intra-arterial catheter, for example, in an acute stroke setting,

Amounts effective for these uses can depend on the severity of the condition, disease or disorder or the severity of the risk of the condition, disease or disorder, and the weight and general state and health of the subject, but generally range from about 0.05 μg to about 1000 μg (e.g., 0.5-100 μg) or 0.5 mg/kg to about 3 mg/kg per body weight per subject of an equivalent amount of the decoy peptide per dose per subject. Suitable regimes for initial administration and booster administrations are typified by an initial administration followed by repeated doses at one or more hourly, daily, weekly, or monthly intervals by a subsequent administration. In some aspects, the decoy peptide can be administered every day. In some aspects, the decoy peptide can be administered once every day. In some aspects, the decoy peptide can be administered every other day. In some aspects, the decoy peptide can be administered once every other day. For example, a subject can receive any of decoy peptides, polypeptides or fragments thereof described herein in the range of about 0.05 to 1,000 μg or to 3.0 mg/kg body weight equivalent dose per dose one or more times per week (e.g., 2, 3, 4, 5, 6, or 7 or more times per week). For example, a subject can receive 0.1 to 2,500 μg (e.g., 2,000, 1,500, 1,000, 500, 100, 10, 1, 0.5, or 0.1 μg) dose per week. In some aspects, a subject can receive 0.5 to 3.0 mg/kg daily (e.g., 3.5 to 21 mg/kg body weight). In some aspects, a subject can receive 0.5 to 3.0 mg/kg (e.g., 3.5 to 21 mg/kg body weight) daily dose per week. In some aspects, a subject can receive 0.5 to 3.0 mg/kg (e.g., 3.5 to 21 mg/kg body weight) per dose for one or more does per day per week. In some aspects, the decoy peptide can be administered as a dose of 0.50 to 2 mg/kg or any amount in between. In some aspects, the decoy peptide can be administered as a dose of 0.50 to 1 mg/kg or any amount in between. In some aspects, the decoy peptide can be administered as a dose of 0.50 to 0.75 mg/kg or any amount in between. In some aspects, the decoy peptide can be administered as a dose of 0.50, 0.55, 0.60, 0.65, 0.70, 0.75, 0.80 0.85, 0.90, 0.95, 1.0, 1.25, 1.50, 1.75 to 2 mg/kg or any amount in between. A subject can also receive any of the decoy peptides, polypeptides or fragments thereof described herein in the range of 0.1 to 3,000 μg per dose once every two or three weeks. In some aspects, a subject can also receive any of the decoy peptides, polypeptides or fragments thereof described herein in the range of 0.5 to 3.0 mg/kg body weight per dose once every two or three weeks. A subject can also receive 2 mg/kg every week (with the weight calculated based on the weight of the decoy peptide or polypeptide described herein and the weight in kg calculated based on the weight of the subject).

The total effective amount of decoy peptide or polypeptide in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, every other day, 1-2 weeks, or once a month). Alternatively, continuous intravenous infusions sufficient to maintain therapeutically effective concentrations in the blood are also within the scope of the present disclosure.

The therapeutically effective amount of one or more of the decoy peptides, polypeptides or fragments thereof present within the compositions described herein and used in the methods as disclosed herein applied to mammals (e.g., humans) can be determined by one of ordinary skill in the art with consideration of individual differences in age, weight, and other general conditions (as mentioned above).

### EXAMPLES

### Example 1: Triglyceride-Lowering Effect

Two groups of Zucker diabetic fatty rats (N=4 each) were treated with alternate daily intraperitoneal dose (IP) of either a composition comprising decoy peptide 2 (SEQ ID NO: 2) (2 mg/kg) or an equal volume of sterile saline (vehicle) for 5 weeks (between weeks 10-16). Blood was obtained at week 16 for determination of plasma hepatic transaminases (AST, ALT) and plasma triglycerides.

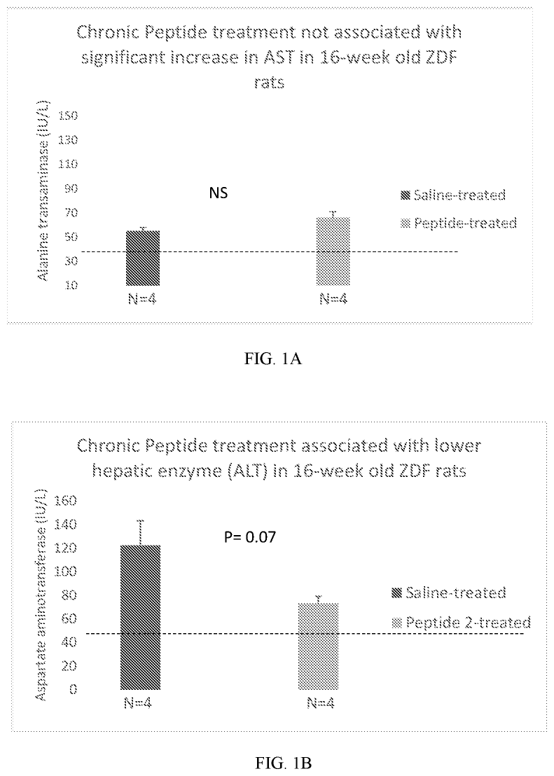

As shown in FIG. 1, mean plasma AST was slightly elevated (1.6-1.9-fold above background level) and did not differ significantly in rats receiving chronic treatment with decoy peptide 2 vs. saline (FIG. 1A). In ZDF rats treated with saline, the mean plasma ALT was 2.7-fold higher than upper limit of the normal range and there was trend of a statistically significant difference compared to decoy peptide 2-treated ZDF rats (FIG. 1B, P=0.07 for the difference). The dashed line indicates the upper level of the normal range for AST and ALT.

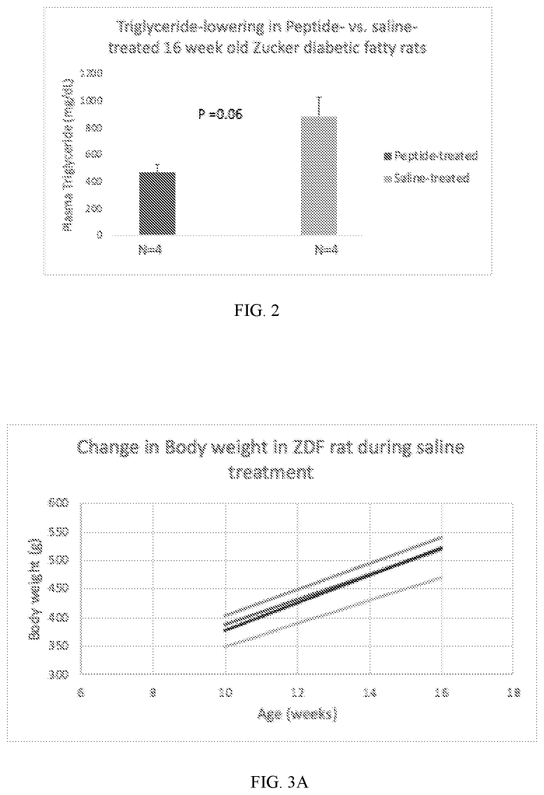

During the same treatment period (week 10-16), there was a nearly significant difference in the mean plasma triglyceride level between peptide- vs saline-treated ZDF rats (FIG. 2; P=0.06). Mean plasma triglyceride level was substantially (47%) lower in peptide-vs. vehicle-treated ZDF rats.

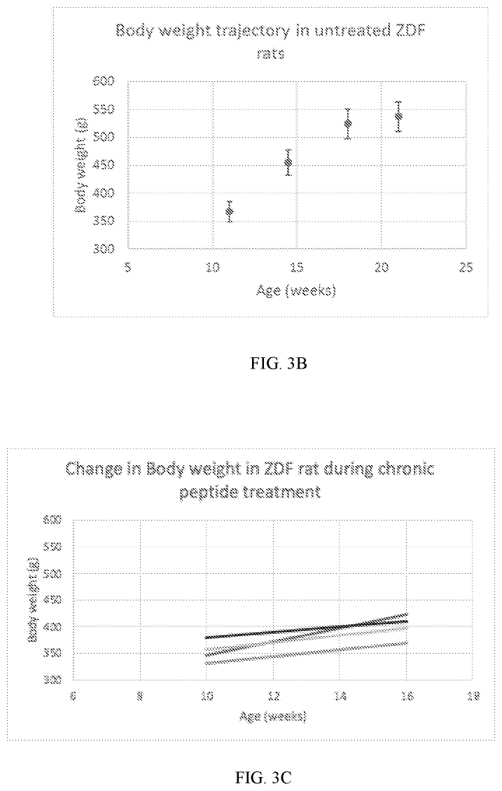

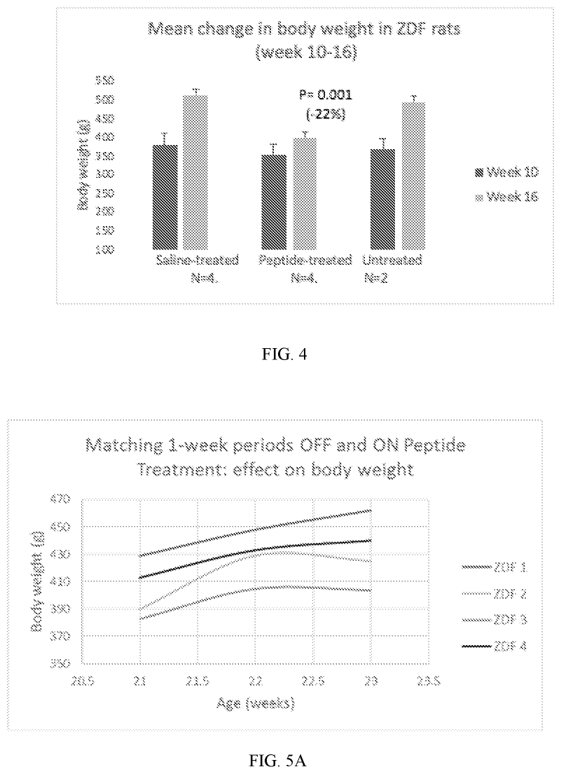

The trajectory of weight change during the same time period (week 10-16) was higher in saline-treated (FIG. 3A) and untreated ZDF rats (FIG. 3B) compared to decoy peptide 2-treated ZDF rats in which the trajectory of weight gain was flatter (FIG. 3C). Both saline-treated and untreated groups exhibited >25% weight gain over the 6-week period (FIG. 4) compared to decoy peptide 2-treated ZDF rats who (at age 16 weeks) had mean body weight which was 22% percent lower than in saline-treated ZDF rats (P=0.001 for the difference, FIG. 4). Mean glucose levels was not significantly different in untreated vs decoy peptide 2-treated ZDF rats (444+/−33 vs 490+/−38 mg/dL) suggesting that weight gain difference between the two groups was not likely attributable to differences in severity of hyperglycemia. The saline-treated ZDF rats had lower mean glucose concentrations (191+/−22 mg/dL).

The underlying causes of reduction in trajectory of body weight gain in decoy peptide 2- vs saline-treated or untreated ZDF rats is unknown. Food consumption was compared during a 24-hour period in matching one-week periods OFF vs ON alternate daily (2 mg/kg) IP injection of decoy peptide 2.

As shown in FIG. 5, the onset of alternate daily decoy peptide 2 injection (arrow) at the start of week 22 was associated with ‘flattening’ of the trajectory of subsequent weight gain in 3 of 4 ZDF rats compared to the trajectory in week 21-22 OFF peptide injection (FIG. 5A). During a matching 24-hour time period in week 22-23 (ON) vs week 21-22 (OFF) decoy peptide 2 injection, there was a highly significant 18.3% reduction in food consumption (44.5 g vs 54.5 g: P=0.007; FIG. 5B) ON vs OFF peptide injections. These data suggest that flattening of the trajectory of weight gain in decoy peptide 2-treated ZDF rats may be due (in part) to acutely lower food consumption via unknown mechanisms.

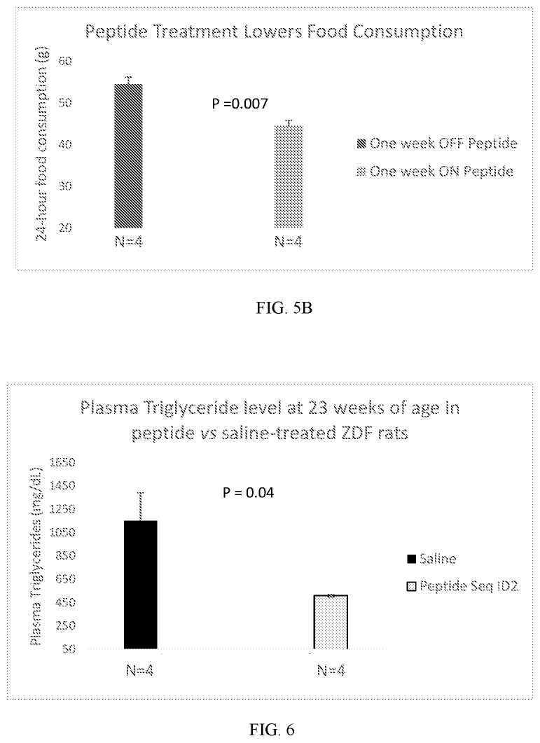

At the end of week 22, blood was drawn for determination of plasma triglyceride level. Mean plasma triglyceride was significantly lower in decoy peptide 2-treated vs. saline-treated ZDF rats (509 vs. 1150 mg/dL; P=0.04; FIG. 6). Total 9 weeks of alternate daily decoy peptide 2 (2 mg/kg) over a 12-week period (week 10-22) was associated with 56% overall reduction in mean triglyceride level.

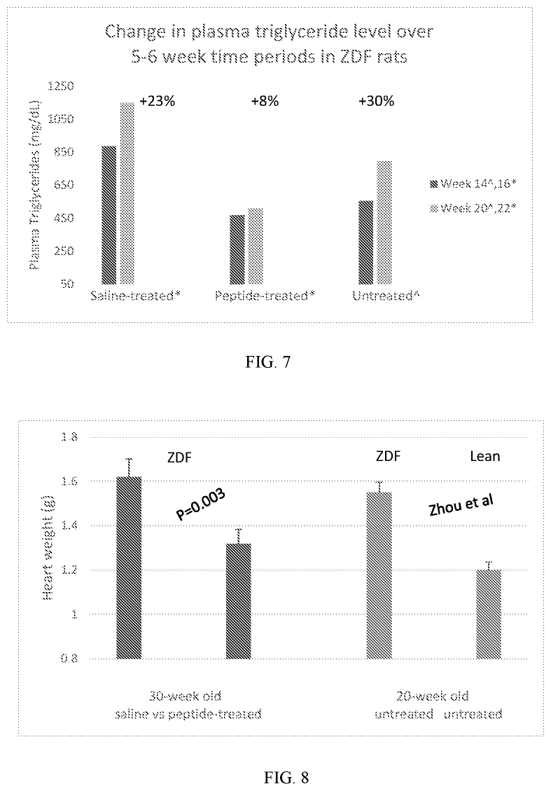

Next, the change in plasma triglyceride levels was compared over a 6-week time period in ZDF rats treated with decoy peptide 2 or saline to data from a control group of untreated ZDF rats reported in the literature [Zhou et al 2000]. Saline-treated ZDF rats experienced a +23% increase in mean plasma triglyceride between week 16-22 compared to peptide-treated ZDF rats who experienced an 8% increase in mean plasma triglycerides (FIG. 7). By comparison, in the study by Zhou et al. control, untreated ZDF rats experienced a +30% increase in mean triglyceride level (FIG. 8) between week 14-20.

In the study by Zhou et al. [2000], ZDF rats were sacrificed at 20 weeks of age and heart weight, heart triglyceride content, and myocardial function was determined in ZDF vs lean Zucker rats.

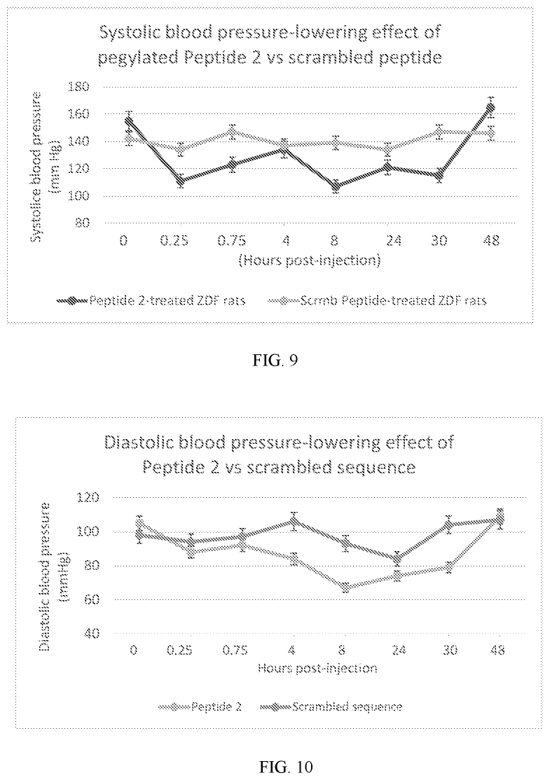

As shown in FIG. 8, heart weight was significantly increased (mean 1.55 g vs. 1.2. g) in ZDF vs lean rats from the study by Zhou et al (2000). In ZDF rats, left ventricular end diastolic chamber diameter was significantly increased indicative of reduced systolic function (i.e., heart failure), and myocardial fractional shortening was reduced. Myocardial tissue contained 3-fold higher content of triglyceride than in age-matched Zucker lean rats [Zhou et al 2000]. In the present study, rats were sacrificed at 30 weeks of age, and mean heart weight was significantly increased (1.62 vs 1.32 g; P=0.003) in saline-treated compared to decoy peptide 2-treated ZDF rats (FIG. 9). Cardiac histology or functional cardiac studies on the ZDF rats was not carried out. Yet taken together with the data from Zhou et al, much higher plasma triglyceride levels in saline-treated ZDF rats from the study described herein may signify that significantly increased heart weights in saline-treated vs decoy peptide 2-treated ZDF rat in the instant study are likely to be associated with cardiac dysfunction due in part to accumulation of triglyceride in myocardial cells causing cellular dysfunction. Two-fold lower plasma triglyceride level in decoy peptide 2-treated ZDF rats suggests a possible cardioprotective role of peptide via substantially reducing plasma triglyceride levels and preventing cardiac steatosis which was associated with functional cardiac impairment in the study by Zhou et al (2000).

In summary, chronic treatment with decoy peptide 2 was associated with blunting of the normal trajectory of weight gain in Zucker diabetic fatty rats which was associated with a substantial reduction in plasma triglyceride level leading to reductions in hepatic steatosis and cardiac weight in decoy peptide 2-treated vs saline-treated Zucker diabetic fatty rats.

The Zucker diabetic fatty rat demonstrates baseline severe hypertriglyceridemia starting as early as age 7 weeks when excess fat mass develops. The mean plasma triglyceride in four, untreated 7-week old ZDF rats was 708+/−155 mg/dL. Normally, the plasma triglyceride level increases as the ZDF rat matures and experiences further increase in fat mass. By age 16 weeks, four ZDF rats treated with vehicle (saline) (starting at 10 weeks of age) had a mean plasma triglyceride level of 886+/−292 mg/dL. On the other hand, four ZDF rats treated with 2 mg/kg decoy peptide 2 on alternate daily dosing schedule for 5 weeks (age 10 wks-16 wks) experienced a 47% reduction in the mean plasma triglyceride level. Chronic decoy peptide 2 treatment was associated with a 2.6-fold greater reduction in plasma triglyceride level than chronic treatment with an FDA-approved potent omega 3 fish oil EPA in humans (47% vs 18%). In humans, sustained weight loss is associated with greater reduction in plasma triglyceride level (up to 40%) than any FDA-approved medication. The data in ZDF rats are consistent with the possibility that sustained slowing of the normal trajectory of weight gain in chronic decoy peptide 2 treated rats is the likely underlying cause for reduction in plasma triglyceride level relative to saline-treated ZDF rats. The results suggest that decoy peptide 2 may be a safe and well-tolerated agent, and its administration can be effective method to promote significant loss of fat mass in obese humans, leading to an even greater risk reduction in cardiovascular disease occurrence in humans than was reported for the use of the most potent FDA-approved, triglyceride-lowering medications.

The data described herein provides proof-of-concept that alternate daily dosing of 2 mg/kg decoy peptide 2 was safe, well-tolerated and associated with substantially greater (47%) reduction in plasma triglyceride level in baseline severe hypertriglyceridemic, obese Zucker rats than previously reported (in humans) for any FDA-approved pharmacologic class of triglyceride-lowering medications (fibrates, omega 3 fish oil, statins).

Summary: Obesity and obese type 2 diabetes mellitus are associated with moderate increase in blood fat levels called triglycerides. High blood fat level was recently shown to be an independent risk factor for premature heart disease including but not limited to: atherosclerosis, atherosclerotic heart disease and stroke occurrence. The American College of Cardiology and the American Heart Association's 2018 Cholesterol Guideline recommends treatment of obesity and metabolic syndrome for adults having fasting or non-fasting blood fat level ranging from triglyceride levels of 175-499 mg/dL. In a recent clinical trial (REDUCE-IT) involving more than 8000 high-risk patients who were already taking a different fat-lowering medication called “statin,” but still had high triglyceride level (135-500 mg/dL), an omega 3 fish oil called EPA reduced the plasma triglyceride level after 1 year treatment by 18.3%. This 18% reduction translated to a 25 percent significant (HR, 0.75; p<0.001) reduction in the occurrences of cardiovascular death, nonfatal heart attack, nonfatal stroke, coronary stenting or coronary bypass or unstable angina (Bhatt D L, et al. J Am Coll Cardiol. 2019; 73(22):2791-2802).

As described herein using a rat model of obesity and type 2 diabetes, it was demonstrated that every-other-daily injection of decoy peptide 2 slowed down rapid weight gain in fatty rats compared to rats that received salt-water injections. Rats who received the peptide had 22% lower body weight after 6 weeks treatment with decoy peptide 2 compared to fatty rats who received salt-water injections. The lower body weight was associated with lower blood fat levels, lower liver enzyme level, and lower heart weight. Since excess blood fat deposits in the heart and liver impairing their function, decoy peptide 2 (by lowering high blood fat levels) may protect the heart and liver from damage due to very high blood fat levels which are common in overweight, obese and adults with type 2 diabetes.

### Example 2: Long-Lasting Blood Pressure-Lowering Effect of Pegylated Peptide SEQ ID NO: 2

Methods. Pegylation of Decoy Peptide 2—SEQ ID NO: 2. The starting material, PEG2K-or-above and DBCO is a mixture of polyethylene glycol (PEG) and the average molecular weight is 1K. The purity of the free peptide is ≥95%. The mechanism for generation of pegylated decoy peptide 2 is as follows:

Lys(Azide)-SCLLADDN (SEQ ID NO: 5)+DSPE-PEG2000 (or above)-DBCO The click chemistry occurs between azide and DBCO. The sequence of the final product is K(DBCO-DSPE-PEG2K)-SCLLADDN (SEQ ID NO: 6). The purity is ≥95% and the MW is 1004.09 g/mol. Scrambled decoy peptide 2 has a sequence of LASNDCLD (SEQ ID NO: 7) and a purity of 96.37%, MW 849.91 g/mol.

Statistical Analysis. Time series analysis was used to test for significant differences between 1 mg/kg pegylated decoy peptide 2 vs 2 mg/kg scrambled decoy peptide 2 sequence. Results. As shown in FIG. 9, a single IP injection of 1 mg/kg pegylated decoy peptide 2 caused a significant decline in mean systolic blood pressure (in 3 ZDF rats). Blood pressure declined from baseline 155 mm Hg to 111 mg Hg (−28%) within 15 minutes of drug administration. Blood pressure remained significantly lower compared to baseline for at least hours after pegylated decoy peptide 2 administration. After 48 hours, systolic blood pressure had returned to its baseline elevated level. A single IP injection of 2 mg/kg scrambled sequence of decoy peptide 2 had no significant effect on baseline systolic blood pressure. Systolic blood pressure remained relatively unchanged over a 48-hour monitoring period after injection (FIG. 9). These data suggest that the systolic blood pressure lowering effect of pegylated decoy peptide 2 is specific for the peptide sequence of decoy peptide 2. In addition, the data demonstrate that a pegylated form of decoy peptide 2 is long-lasting: providing normalization of systolic blood pressure for at least 30 hours after administration. The long lasting effect may prove useful as a tool in the treatment of acute severe hypertensive states in the emergency room or in a short-term observation unit perhaps obviating the need for costly inpatient hospitalization, allowing earlier discharge of patients while ensuring long-lasting effectiveness of blood pressure lowering in the days following initial presentation.

Pegylated decoy peptide 2 (1 mg/kg) caused a significant reduction in diastolic blood pressure in ZDF rats. Diastolic blood pressure was reduced by 20% at 4 hours post-injection and it reached a maximum of 36% reduction 8 hours after injection. It was still 34% lower than baseline at 30 hours post injection (FIG. 10). These data are consistent with the long-lasting results of systolic blood pressure-lowering in response to pegylated decoy peptide 2. In contrast, IP injection of 2 mg/kg of scrambled sequence of decoy peptide 2 had no significant effect on diastolic blood pressure in ZDF rats (FIG. 10).

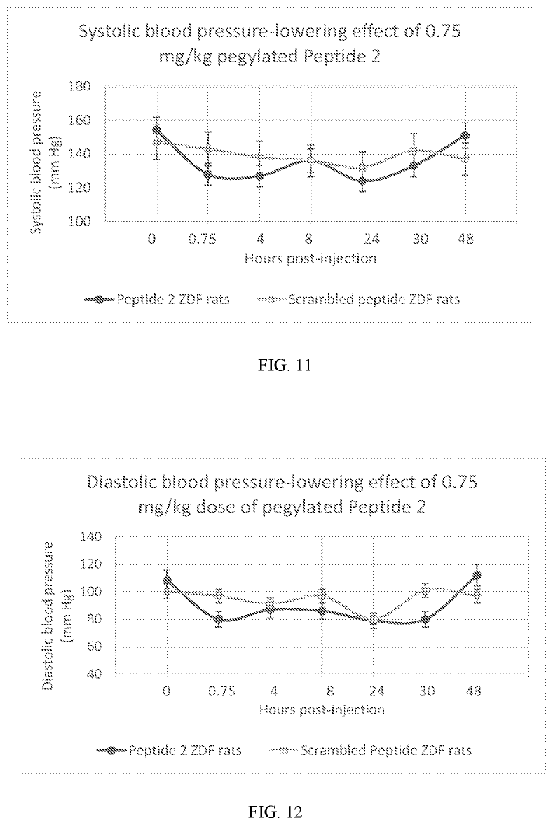

Next, a lower IP dose of pegylated decoy peptide 2 (0.75 mg/kg) was tested for effects on blood pressure lowering in ZDF rats. As shown in FIG. 11, peglyated decoy peptide 2 (0.75 mg/kg) caused a significant (17%) reduction (from 154 mm Hg to 128 mm Hg) at the 45 minutes (0.75 hour) post-injection time point. Systolic blood pressure lowering was long-lasting: 14-19% reduction was still observed between 24-30 hours after a single IP injection. Systolic blood pressure recovered to its baseline level 48 hours after injection of pegylated decoy peptide 2. There was no significant systolic blood pressure lowering effect of a single 2 mg/kg IP dose of scrambled decoy peptide 2 sequence (FIG. 11). As shown in FIG. 12, a single IP dose of 0.75 mg/kg pegylated decoy peptide 2 caused significant 19% diastolic blood pressure lowering in ZDF rats (from baseline 108 mm Hg to 80 mm Hg) at the 45 minutes post-injection time point. Diastolic blood pressure remained decreased to 80 mm Hg 30 hours post injection and it recovered to baseline level 48 hours post-injection. There was no consistent diastolic blood pressure lowering effect in response to IP injection of 2 mg/kg scrambled decoy peptide 2 sequence.

Taken together, these data demonstrate significant, dose-dependent systolic and diastolic blood pressure lowering effects of pegylated decoy peptide 2.

TABLE 1
Comparison of systolic blood pressure lowering effect from pegylated decoy
peptide 2 (1 mg/kg IP) vs scrambled decoy peptide 2 sequence (2 mg/kg IP).

| | | | Standard | | | |
|---|---|---|---|---|---|---|
| Effect | Treatment | Estimate | Error | DF | t Value | Pr > \|t\| |
| Intercept | | 141.66 | 5.4602 | 5 | 25.94 | <0.0001 |
| Trt | Peg decoy peptide 2 | −8.9162 | 2.5229 | 38 | −3.53 | 0.0011 |
| Trt | Scrambled decoy | 0 | | | | |
| | peptide 2 | | | | | |

TABLE 2
Comparison of diastolic blood pressure lowering effect from pegylated decoy
peptide 2 (1 mg/kg IP) vs scrambled decoy peptide 2 sequence (2 mg/kg IP).

| | | | Standard | | | |
|---|---|---|---|---|---|---|
| Effect | Treatment | Estimate | Error | DF | t Value | Pr > \|t\| |
| Intercept | | 98.1654 | 4.3760 | 5 | 22.43 | <0.0001 |
| Trt | Peg decoy | −9.7771 | 0.5121 | 40 | −19.09 | <0.0001 |
| | peptide 2 | | | | | |
| Trt | Scrambled | 0 | | | | |
| | decoy peptide 2 | | | | | |

As shown in Table 1, the difference in mean systolic blood pressure lowering in the ZDF rat treated with pegylated decoy peptide 2 (1 mg/kg) vs. scrambled decoy peptide 2 (2 mg/kg) was statistically significant (<0.001). Pegylated decoy peptide 2 caused mean −8.9 mm Hg difference in mean systolic blood pressure compared to scrambled decoy peptide 2.

As shown in Table 2, the difference in mean diastolic blood pressure lowering in the ZDF rat treated with pegylated decoy peptide 2 (1 mg/kg) vs. scrambled decoy peptide 2 (2 mg/kg) was statistically significant (<0.001). Pegylated decoy peptide 2 caused mean −9.8 mm Hg difference in mean diastolic blood pressure compared to scrambled decoy peptide 2.

The value of the intercept in Tables 1 and 2 corresponds to the baseline mean blood pressure before drug (e.g., peglyated decoy peptide 2 or scrambled decoy peptide 2) administration.

### Example 3: Pegylated Peptide SEQ ID NO: 2 Reduces and/or Prevents Cardiac Hypertrophy Associated with Chronic Moderate-Severe Hypertension in the ZDF Rat

Obesity-associated hypertension is a major risk factor for adverse cardiovascular and renal outcomes in adult type 2 diabetic populations (Landsberg L, et al. Obesity (Silver Spring). 2013 January; 21(1):8-24; and Hall J, et al. Curr Opin Nephrol Hypertens 2003; 12: 195-200). The underlying pathophysiologic mechanisms are complex and may include inflammation, lipotoxicity, and endothelial cell dysfunction as contributory factors (Landsberg L, et al. Obesity (Silver Spring). 2013 January; 21(1):8-24). Obesity is thought to drive sympathetic nervous system overactivation in the kidney (Esler M, et al. Am J Hypertens. 2001 November; 14(11 Pt 2):3045-3095) contributing to hypertension and the development of left ventricular hypertrophy (Woodiwiss A. J., and Norton G. R. Curr. Hypertens Rep. 2015; 17:539). Left ventricular hypertrophy is a risk factor for heart failure and myocardial infarction which both increase substantially in adult obese, hypertensive type 2 diabetes mellitus (Kenny H C and Abel E D. Circ Res. 2019; 124(1):121-141; and Adlerberth A M, et al. Diabetes Care 1998; 21:539-545).

Volume and pressure overload cardiac hypertrophy is driven by catecholamines (Rossi M A and Carillo S V. Int J Cardiol. 1991; 31(2):133-141) and other hormones (e.g. angiotensin II) which (in the case of alpha 1β adrenergic R) activates Gq/phospholipase C-coupled signaling pathways in cardiac cells (Eckhart A D, et al. Circ Res. 2000; 86(1):43-50). A hallmark feature of classical ligand-G-protein receptor interaction(s) is ligand-occupied receptor desensitization (via phosphorylation) mediated by G-protein coupled receptor kinases (GRKs) (Eckhart A D, et al. Circ Res. 2000; 86(1):43-50). Receptor autoimmunity, on the other hand, is characterized by humoral IgG receptor-targeting autoantibodies (e.g., TSH receptor or beta-adrenergic receptor) that elicit longer-lasting receptor activation underlying various human pathologies such as Graves' disease (Adams D D. Autoimmunity. 1988; 1(1):3-9) or dilated cardiomyopathy, respectively (Jahns R, et al. J Clin Invest. 2004; 113(10):1419-1429). Recently, it was reported that subsets of human diabetic microvascular disease, stroke, refractory hypertension and/or chronic kidney disease harbored increased plasma IgG, 5-hydroxytryptamine 2A receptor (5-HT2AR)-targeting autoantibodies that caused long-lasting Gq11/phospholipase C/Ca2+ signaling activation in endothelial cells and in neurons (Zimering M B. J Endocrinol Diabetes. 2017; 4(4):10; Zimering M B. J Endocrinol Diabetes. 2018; 5(2):10; and Zimering M B. Endocrinol Diabetes Metab J. 2019; 3(4):118).

The 5-HT2AR is expressed on arterial vascular smooth muscle cells where it mediates induced arterial vasoconstriction (Watts S W, et al. Pharmacol Rev. 2012; 64(2):359-88). A decoy peptide comprising a subregion of the second extracellular loop of the 5-HT2A receptor involved in mediated long-lasting receptor activation (Wacker D, et al. (2017) Cell 168: 377-389) was developed. The decoy peptide 2, SCLLADDN (SEQ ID NO: 2) prevented human 5-HT2AR-targeting IgG autoantibodies' endothelial and neuronal cell toxicity in vitro (Zimering M B. Endocrinol Diabetes Metab J. 2019; 3(4):118). The aim of the present study was to test whether the decoy peptide 2 (SEQ ID NO: 2) acutely lowers blood pressure in an animal model of obesity-associated hypertension which harbors spontaneously-occurring plasma 5-HT2AR agonist autoantibodies (Zimering M B, et al. Endocrinol Diabetes Metab J. 2020; 4(3):413).

The male Zucker diabetic fatty rat (ZDF) is a well-known genetic model of obese, hypertensive, dyslipidemic type 2 diabetes mellitus (Kurtz T W, e al. Hypertension 1989; 13: 896-901; and Clark J B, et al. Proc Soc Exp Biol Med 1983; 173(1): 68-75). In the ZDF rat, agonist plasma 5-HT2AR targeting IgG autoantibodies appeared to develop around the same time as obesity and diabetes and caused persistent Gq11/PLC/Ca2+ signaling in cells (Zimering M B, et al. Endocrinol Diabetes Metab J. 2020; 4(3):413). It was tested whether chronic administration of decoy peptide 2 vs. scrambled peptide 2 (for ˜13 weeks) might protect against deleterious cardiac hypertrophy associated with chronic moderate-severe hypertension in the ZDF rat.

Methods. Synthetic peptides. The synthetic peptides were synthesized at Lifetein Inc. (Hillborough, NJ). The lyophilized peptides were aliquoted and stored (in the presence of desiccant) at −40° C. prior to use. On the day of intraperitoneal (IP) administration, an aliquot of lyophilized peptide was reconstituted in sterile saline at the appropriate concentration ranging from 0.75 mg/kg to 2 mg/kg. Reconstituted peptide was prepared fresh before each injection. Lyophilized peptide was stored for up to 4 weeks (at −40° C.) prior to obtaining newly-synthesized peptide needed in chronic drug administration experiments.

Decoy peptide 2. A linear synthetic peptide, SCLLADDN (SEQ ID NO: 2), was synthesized and had >95% purity.

Pegylated decoy peptide 2. The starting material, PEG2K-or-above DBCO is a mixture of polyethylene glycol (PEG) and the average molecular weight is 1K. The purity of the free peptide is >95%. The mechanism for the generation of pegylated decoy peptide 2 is as follows: Lys(Azide)-SCLLADDN (SEQ ID NO: 5)+DSPE-PEG2000 (or above)-DBCO. The click chemistry occurs between azide and DBCO. The sequence of the final product is K(DBCO-DSPE-PEG2K)-SCLLADDN (SEQ ID NO: 6). The purity is >95% and the MW is 1004.09. Scrambled Peptide sequence LD.8 (SEQ ID NO: 7). The scrambled decoy peptide 2 had a sequence of LASNDCLD (SEQ ID NO: 7) and a purity of 96.37%, MW 849.91 g/mol.

Animals. Male ZDF and lean (+/?) Zucker rats were obtained from Charles River Laboratories (Kingston, NY) at approximately 6-7 weeks of age. The rats were single housed upon arrival, without enrichment. Rats were acclimatized for two weeks prior to experimental procedures. Rats were provided ad libitum access to food and water and maintained in a 12 h light/dark cycle with lights on at 0630. The procedures occurred during the light phase of the cycle. Blood pressure testing (following acute or chronic administration of the decoy peptides) was conducted in three separate cohorts of animals.

Cohort 1: Three 25-week-old male ZDF and three, age-matched, male Zucker lean rats (ZLR) treated with IP decoy peptide 2 (ZDF) vs saline (vehicle) (ZLR).

Cohort 2: Eleven-week old male ZDF (n=5) and Zucker lean rats (n=4) treated with IP decoy peptide 2.

Cohort 3: Twelve 8.5-week old male Zucker diabetic fatty rats were randomly assigned to one of three treatment groups (n=4/group). The rats underwent baseline blood pressure determinations using tail-cuff plethysmography technique. Next, the animals who expressed baseline hypertension, i.e., blood pressure >135/85, received either a 1 mg/kg intraperitoneal injection of pegylated decoy peptide 2 (n=3); a 2 mg/kg intraperitoneal dose of a scrambled decoy peptide 2 (n=4); or vehicle (i.e., sterile saline) (N=4). Blood pressure was determined at 15 minutes, 45 minutes, 4 hours, 8 hours, 24 hours, 30 and 48 hours following single IP injection. One week later, the same two groups of ZDF rats received either 0.75 mg/kg IP pegylated decoy peptide 2, 2 mg/kg scrambled decoy peptide 2, or sterile saline; and blood pressure was determined at similar timepoints. Two weeks later, two groups of 12-week-old ZDF rats (n=6/group) including those that had previously received acute peglyated decoy peptide 2 or scrambled decoy peptide 2 were treated on an alternative daily schedule with IP, free decoy peptide 2 (2 mg/kg) vs IP scrambled decoy peptide 2 (2 mg/kg) continuously for 13 weeks until sacrifice at 25 weeks of age. Body weight was determined immediately before sacrifice and after perfusion; and hearts were excised and weighed for determination of the heart-to-(perfused) body weight ratio.

Blood pressure monitoring. Tail cuff blood pressure measurement was performed using an automated CODA noninvasive blood pressure system (Kent Scientific, Torrington, CT). Rats were placed on an insulated warming platform in a well-heated room to ensure proper body temperature. Cuff monitors were provided in different sizes to accommodate rats of different ages and having different body weights. After the animal was positioned in the clear plastic restraint holder, the appropriate-sized cuff was slid over the rat's tail. The blood pressure system uses volume pressure recording tail-cuff technology and displays up to six blood pressure measurements per cycle.

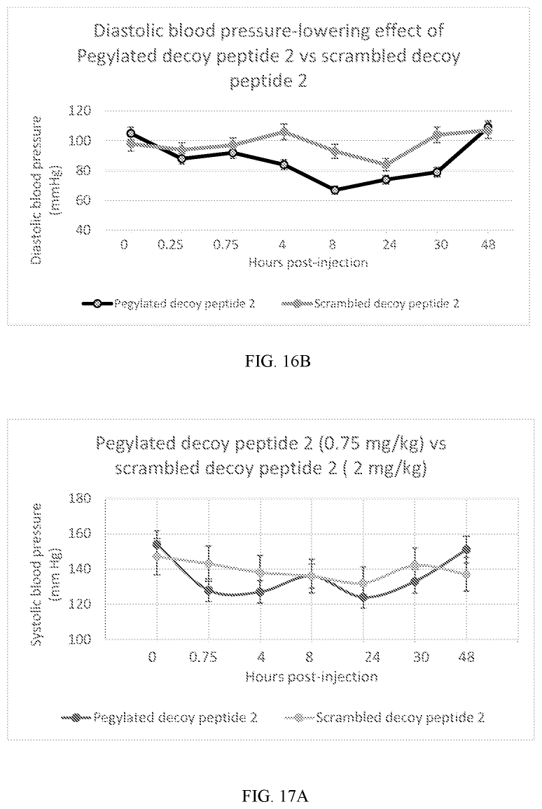

Statistical Analysis. Comparisons were made using Student's unpaired t-test (Tables 3-6, 9 and FIG. 18) or time series analysis (Tables 7, 8, FIG. 16).

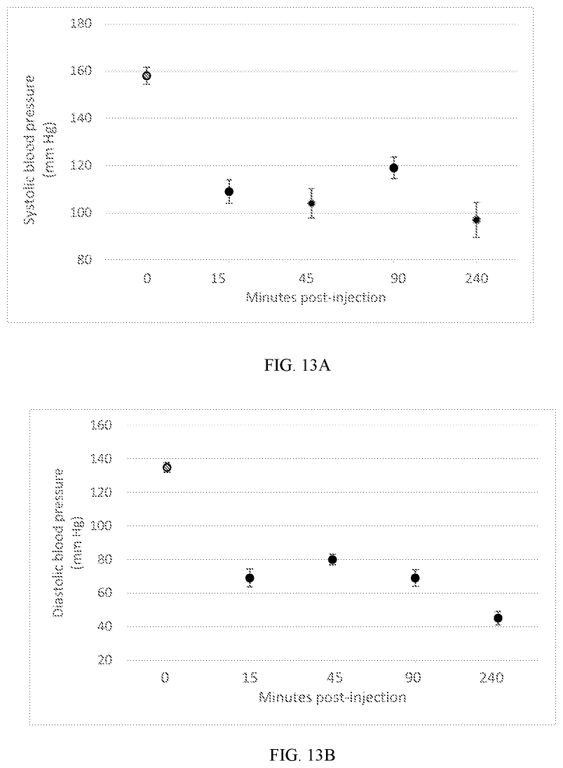

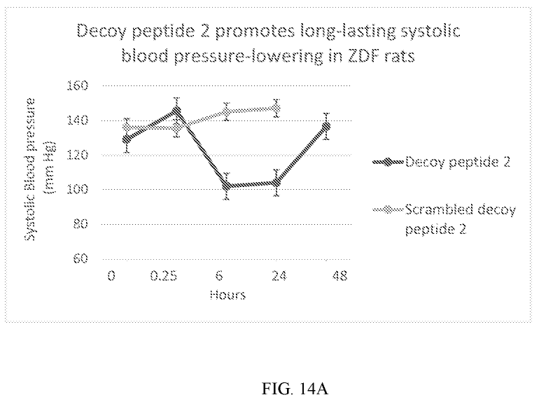

Results. Acute blood pressure-lowering effect of decoy peptide 2 (2 mg/kg IP) in male ZDF rats. The male ZDF rat spontaneously develops hypertension by age 8-10 weeks, and manifests proteinuric nephropathy by approximately 18 weeks of age (Coimbra T, et al. Kidney Int 57: 167-182, 2000). Decoy peptide 2 is a linear synthetic peptide having an amino acid sequence SCLLADDN (SEQ ID NO: 2) corresponding to a region of the second extracellular loop of the 5-HT2A receptor involved in mediating long-lasting activation (Wacker D, et al. (2017) Cell 168: 377-389). A single 2 mg/kg IP dose of decoy peptide 2 caused highly significant (41-42%) acute decreases in systolic and diastolic blood pressure in three 25-week-old male ZDF rats tested, rats that were naïve to prior drug exposure (Table 3). The onset of acute blood pressure-lowering occurred between 15-40 minutes following IP drug injection. In two of three rats tested, IP decoy peptide 2 administration caused sedation and borderline hypotension both of which resolved spontaneously after approximately ten minutes. Rechallenge (1 week later) with a single IP (2 mg/kg) dose of decopy peptide 2 caused reproducible large drops in systolic and diastolic blood pressure which were sustained for 4 hours or longer (FIG. 13). There were no untoward acute or long-term side effects observed for up to 18 days following the repeat drug exposure. Taken together, these data demonstrate that a single 2 mg/kg intraperitoneal injection of the decoy peptide 2 (dissolved in sterile isotonic saline) causes acute substantial lowering of systolic, diastolic and mean arterial blood pressure in the older adult 25-week-old, hypertensive Zucker diabetic fatty rats.

TABLE 3
Change in systolic blood pressure in 25-week-old male
ZDF rats before and after decoy peptide 2 injection.

| | | | | |
|---|---|---|---|---|
| | | Day 0, | 15-40 mins | |
| Animals | Day 2 | pre-injection | post-injection. | P-value* |

SYSTOLIC BLOOD PRESSURE

| | | | | |
|---|---|---|---|---|
| ZDF 1-3 | 166 ± 24 | 161 ± 21 | 95 ± 13 | 0.018 |

DIASTOLIC BLOOD PRESSURE

| | | | | |
|---|---|---|---|---|
| ZDF 1-3 | 101 ± 23 | 112 ± 2 | 65 ± 11 | 0.05 |

Percent lowering of systolic blood pressure after peptide (161-95)/161 = 41
Percent lowering of diastolic blood pressure after peptide (112-65)/112 = 42
Percent lowering of mean arterial pressure (125-75)/125 = 40
Results are mean mm Hg +/− SD in three, obese male Zucker diabetic fatty rats (25-weeks- old), average weight approximately 500 g who had blood pressure monitored two days before, 30 minutes before and then 15-40 minutes after receiving 2 mg/kg intraperitoneal injection of decoy peptide 2 in sterile saline.
*P-value is comparing mean systolic or diastolic blood pressure immediately before and after the peptide injection.

Acute effect of IP administration of saline in older male Zucker lean rats. Age-matched male Zucker lean rats do not manifest diabetes, obesity or hypertension when fed the same diet as ZDF rats. Baseline mean systolic and diastolic blood pressure was in the normal range in 25-week-old male ZLR rats (N=3), and it did not change significantly following IP administration of 0.5 mL vehicle (sterile saline) in each rat (Table 4).

Table 4. Effect of saline injection on systolic blood pressure in Zucker lean, non-obese rats (ZLR)

TABLE 4
Effect of saline injection on systolic blood
pressure in Zucker lean, non-obese rats (ZLR)

| | | | |
|---|---|---|---|
| Animals | Before | 15-40 minutes after saline injection. | P-value* |

SYSTOLIC BLOOD PRESSURE

| | | | |
|---|---|---|---|
| ZLR 1-3 | 137 ± 10 | 126 ± 26 | 0.72 |

DIASTOLIC BLOOD PRESSURE

| | | | |
|---|---|---|---|
| ZLR 1-3 | 88 ± 5 | 83 ± 0 | 0.42 |

Intraperitoneal injection of 0.5 mL of sterile saline had no significant effect on diastolic blood pressure in three Zucker lean male rats, 25-weeks-old.
*P-value comparing mean systolic and diastolic blood pressure before and after saline injection.

Acute effect of decoy peptide 2 in younger male ZDF and Zucker lean rats. Next, a 2 mg/kg IP dose of decoy peptide 2 in 11-week-old male ZDF rats (N=5), and age-matched male Zucker lean rats (N=4) was tested. Decoy peptide 2 caused acute significant mean systolic and diastolic blood pressure-lowering (19-23%) in five of five 11-week-old male ZDF rats tested (Table 5). Acute blood pressure-lowering was well-tolerated; none of the five male ZDF rats experienced any untoward side effects including hypotension or acute sedation. Three of four 11-week-old Zucker lean rats tested had normal blood pressure at baseline and decoy peptide 2 (2 mg/kg IP) did not significantly alter blood pressure acutely in normotensive rats (Table 6). In one of four Zucker lean rats that manifested baseline hypertension, a single dose of decoy peptide 2 (2 mg/kg IP) acutely lowered systolic and diastolic blood pressure to statistically significantly lower levels compared to baseline (Table 6). These data suggest that 2 mg/kg IP decoy peptide 2 effectively lowers blood pressure in both young and older male ZDF rats and in the ZLR rat the effect may be selective for a subset of hypertensive rats.

TABLE 5
Acute blood pressure- lowering effect of decoy
peptide 2 in 11-week-old male ZDF rats

| | | | |
|---|---|---|---|
| Animals | Before | 15-45 mins post-injection | P-value* |

SYSTOLIC BLOOD PRESSURE

| | | | |
|---|---|---|---|
| ZDF (N = 5) | 167 +/− 24 (N = 10) | 135/−+18 (N = 17) | 0.0004 |

DIASTOLIC BLOOD PRESSURE

| | | | |
|---|---|---|---|
| ZDF (N = 5) | 118 + 22 (N = 10) | 91 + 15 (N = 17) | 0.002 |

Results are mean +/− SD
Mean acute SBP-lowering (167-135)/167 = 19%
Mean acute DBP-lowering (118-91)/118 = 23%
Mean acute MAP-lowering (134-106)/134 = 21%
*P-value: comparing mean systolic or diastolic blood pressure before and after IP injection of 2 mg/kg dose of decoy peptide 2.

TABLE 6
Acute effects of decoy peptide 2 on blood
pressure in 11-week-old Zucker lean rats

| | Blood pressure | | P- |
|---|---|---|---|
| | Before injection | 30 minutes after injection | value* |
| Zucker lean #1 | 130/72 | 120/78 | NS |
| Zucker lean #2 | 124/80 | 122/83 | NS |
| Zucker lean #3 | 130/85 | 132/98 | NS |
| Zucker lean #4 | 149/102 (N = 6) | 130/91 (N = 4) | <0.05 |

*P-value comparing mean blood pressure before and after IP injection of 2 mg/kg decoy peptide 2.

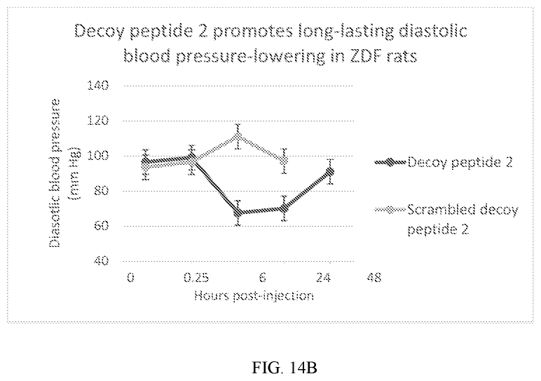

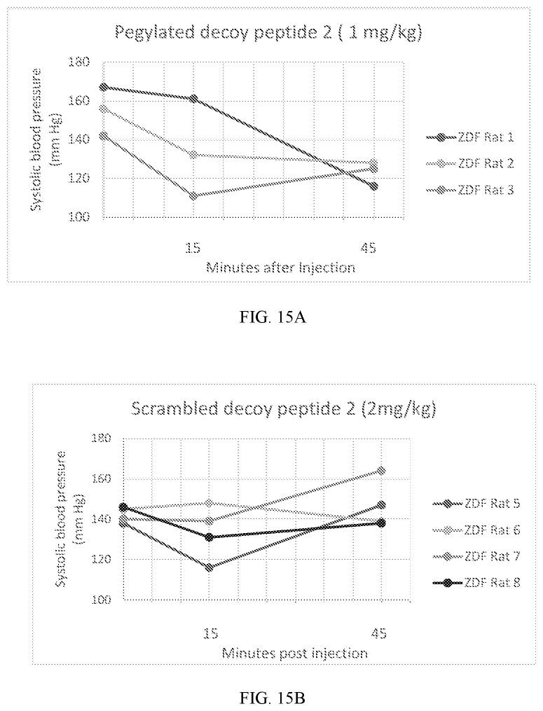

Sustained blood pressure lowering effect of decoy peptide 2 in aged male ZDF rat. The duration of decoy peptide 2's blood pressure-lowering action was evaluated in 25-week-old male ZDF rats (N=3) administered a single IP 2 mg/kg dose. The control group were age matched male ZDF rats (N=3) who received 2 mg/kg IP administration ofa scrambled decoy peptide 2 comprised of the same eight amino acids as in decoy peptide 2 but arranged in a random order. Decoy peptide 2 caused 25-30% significant systolic and diastolic blood pressure-lowering which was sustained for 24 hours or longer (FIG. 14A). Blood pressure returned to baseline elevated levels 48 hours after decoy peptide 2 administration (FIG. 14A). The scrambled decoy peptide 2 (2 mg/kg IP) had no significant systolic or diastolic blood pressure-lowering effect at time points up to 24 hours (FIG. 14B). These data show that a single 2 mg/kg decoy peptide 2 dose (but not scrambled decopy peptide 2) promotes relatively long-lasting significant systolic and diastolic blood pressure-lowering in older hypertensive male ZDF rats.

Effect of pegylated decoy peptide 2 on systolic and diastolic blood pressure in male ZDF rats. Pegylation, the attachment of polyethylene glycol moieties to a peptide or protein, can increase the effective size, circulation time and protect against proteolytic degradation of therapeutic proteins and peptides (Werle M and Bernkop-Schnürch A. Amino Acids. 2006 June; 30(4):351-67). The blood pressure-lowering effects and duration of action was tested in two different (lower concentrations) of pegylated decoy peptide 2.

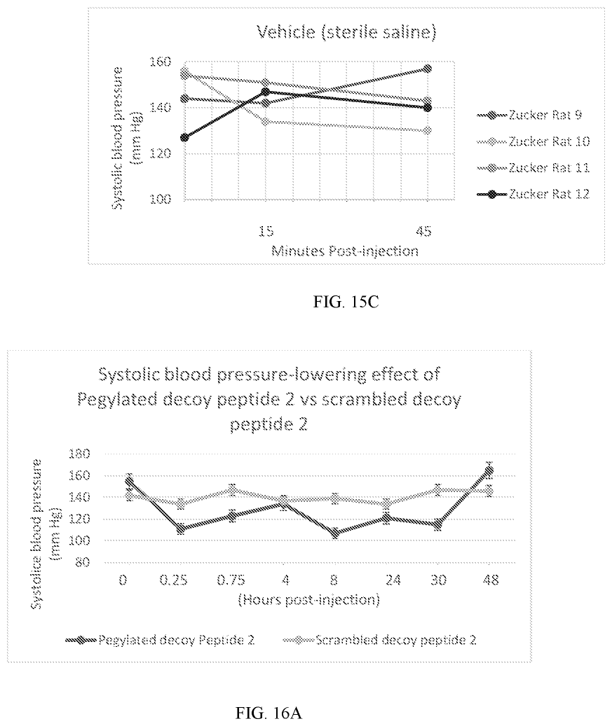

Acute blood pressure-lowering effects from IP administration of 1 mg/kg pegylated decopy peptide 2 was compared to 2 mg/kg (unpegylated) scrambled decoy peptide 2 or 0.5 mL vehicle (sterile saline) in three groups of 8.5-week-old male ZDF rats (N=4 per group). Pegylated decoy peptide 2 (1 mg/kg IP) caused acute significant decline in mean systolic blood pressure compared to baseline (155+/−12 vs 122+/−5 mm Hg; N=3; P=0.013; FIG. 15A), 45 minutes post-injection. One of four animals was excluded from testing because it did not manifest baseline hypertension. Scrambled decoy peptide 2 (2 mg/kg) had no significant systolic blood pressure-lowering effect compared to baseline (142+/−3 vs. 147+/−12; N=4; P=0.44: FIG. 15B) 45 minutes post-injection. Sterile saline (0.5 mL) did not significantly alter systolic blood pressure compared to baseline (145+/−13 vs. 143+/−11; N=4; P=0.80; FIG. 15C) 45 minutes post-injection. Pegylated decoy peptide 2 caused a trend of significant acute decline in diastolic blood pressure 45 minutes post-injection (105+/−8 vs 92+/−11 mm Hg; N=3, P=0.2). There was no significant change in mean diastolic blood pressure 45 minutes following IP injection of either scrambled decoy peptide 2 (99+/−4 vs 104+/−9 mm Hg; N=4; P=0.40) or sterile saline (104+/−15 vs 103+/−13; N=4; P=0.90).

Long-lasting blood pressure-lowering effect of pegylated decoy peptide 2. A single IP injection of 1 mg/kg pegylated decoy peptide 2 caused a significant and long-lasting decline in mean systolic blood pressure in three male (8.5-week-old) ZDF rats. Blood pressure declined from baseline 155 mm Hg to 111 mg Hg (−28%) within 15 minutes of drug administration. Blood pressure remained significantly lower compared to baseline for at least hours after pegylated decoy peptide 2 administration (black line, FIG. 1A). After 48 hours, systolic blood pressure had returned to its baseline elevated level. By comparison, a single IP injection of 2 mg/kg scrambled decoy peptide 2 had no significant effect on baseline systolic blood pressure (grey line, FIG. 16A). Systolic blood pressure remained relatively unchanged over a 48-hour monitoring period after injection (FIG. 16A). Time series analysis revealed a highly significant (P=0.0011) superior systolic blood pressure-lowering effect from 1 mg/kg pegylated decoy peptide 2 vs 2 mg/kg scrambled decoy peptide (Table 7). The value of the intercept in Table 7 corresponds to the baseline mean blood pressure before drug (e.g., peglyated decoy peptide 2 or scrambled decoy peptide 2) administration.

TABLE 7
Comparison of systolic blood pressure-lowering effects of pegylated decoy
peptide 2 (1 mg/kg) vs scrambled decoy peptide 2 (2 mg/kg)
intraperitoneal injection.

| Treatment | | | | | | |
|---|---|---|---|---|---|---|
| Effect | | Estimate | SE | DF | t Value | Pr > t |
| Intercept | | 141.66 | 5.4602 | 5 | 25.94 | <.0001 |
| Trt | Pegylated decoy | −8.9162 | 2.5229 | 38 | −3.53 | 0.0011 |
| | peptide 2 | | | | | |
| Trt | Scrambled decoy | 0 | | | | |
| | peptide 2 | | | | | |

Single pegylated decoy peptide 2 (1 mg/kg) IP injection caused −8.9 mm Hg reduction in mean systolic blood pressure compared to scrambled decoy peptide 2 (2 mg/kg IP).

SE—Standard Error

Pegylated decoy peptide 2 (1 mg/kg) caused a significant long-lasting reduction in diastolic blood pressure in male ZDF rats. Diastolic blood pressure was reduced by 20% at 4 hours post-injection and it reached a maximum of 36% reduction 8 hours after injection (FIG. 16B). It was still 34% lower than baseline 30 hours post injection (FIG. 16B). Time series analysis revealed a highly significant (P<0.001) and superior diastolic blood pressure-lowering action of pegylated decoy peptide 2 (1 mg/kg) vs. scrambled decoy peptide 2 (2 mg/kg) (Table 8).

TABLE 8
Comparison of diastolic blood pressure lowering effect from pegylated decoy
peptide 2 (1 mg/kg IP) vs scrambled decoy peptide 2 (2 mg/kg IP).

| Treatment | | | | | | |
|---|---|---|---|---|---|---|
| Effect | | Estimate | SE | DF | t Value | Pr > t |
| Intercept | | 98.1654 | 4.3760 | 5 | 22.43 | <.0001 |
| Trt | Pegylated decoy | −9.7771 | 0.5121 | 40 | −19.09 | <.0001 |
| | peptide 2 | | | | | |
| Trt | Scrambled decoy | 0 | | | | |
| | peptide 2 | | | | | |

Single pegylated decoy peptide 2 (1 mg/kg) intraperitoneal (IP) injection caused −9.8 mm Hg reduction in mean diastolic blood pressure compared to IP injection of scrambled decoy peptide 2 (2 mg/kg). SE—standard error

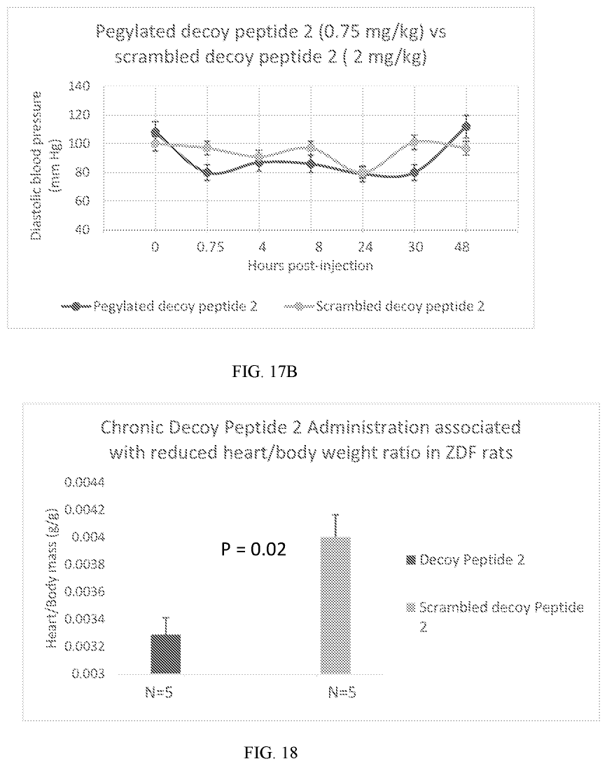

Next, a lower IP dose of pegylated decoy peptide 2 (0.75 mg/kg) was tested for its effects on blood pressure-lowering in ZDF rats. Pegylated decoy peptide 2 (0.75 mg/kg) caused a significant 17% reduction (from 154 mm Hg to 128 mm Hg) at the 45 minutes (0.75 hour) post-injection timepoint (FIG. 17A). Systolic blood pressure lowering was long-lasting: 14-19% reduction was still observed between 24-30 hours after a single IP injection. Systolic blood pressure recovered to its baseline level 48 hours after injection of pegylated decoy peptide 2 (FIG. 17A). No significant systolic blood pressure-lowering effect was observed after administration of a single 2 mg/kg IP dose of scrambled decoy peptide 2 (FIG. 17A).

A single IP dose of 0.75 mg/kg pegylated decoy peptide 2 caused significant 19% diastolic blood pressure lowering in ZDF rats (from baseline 108 mm Hg to 80 mm Hg at the 45 minutes post-injection timepoint) (FIG. 17B). Diastolic blood pressure remained decreased to 80 mm Hg 30 hours post injection and it recovered to baseline level 48 hours post-injection. There was no consistent diastolic blood pressure-lowering effect in response to IP injection of 2 mg/kg scrambled decoy peptide 2 (FIG. 17B). Taken together, single IP administration of pegylated decoy peptide 2 (at doses ranging from 0.75 to 1 mg/kg) caused significant, dose-dependent, long-lasting systolic and diastolic blood pressure-lowering effect in the ZDF rat.

Effects of chronic administration of decoy peptide 2 in male ZDF rats. Obesity, hyperinsulinemia and hypertension have been reported to be associated with cardiomyocyte and cardiac hypertrophy in Zucker diabetic fatty compared to Zucker lean rats (Fredersdorf S, et al. Cardiovasc Pathol. 2004; 13(1):11-19).

Two groups of 15-week-old male Zucker fatty rats (n=6/group) having matching baseline mean capillary glucose concentration and body weight (Table 9) were randomly assigned to chronic 10 weeks' treatment (between weeks 15 and 25) with either alternative daily decoy peptide 2 (2 mg/kg) or 2 mg/kg dose of scrambled decoy peptide 2. These animals were part of a neuroprotection experiment and at 14-weeks of age, half in each drug treatment group (n=3) experienced mild traumatic brain injury (via lateral fluid percussion) or sham injury. One rat in each drug assignment group (assigned to mild traumatic brain injury) failed to gain significant weight post-injury and was excluded from the analysis. In the remaining 10 rats (n=5/decoy peptide 2 group) 10 weeks' IP treatment with decoy peptide 2 was associated with a significantly lower mean heart-to-body weight ratio (3.3 mg/g vs 4.0 mg/g; P=0.02, FIG. 18) compared to scrambled decoy peptide 2 treatment. These data suggest that chronic administration of decoy peptide 2 may have favorably affected moderate-severe hypertension leading to reduced cardiac hypertrophy in male ZDF rats.

TABLE 9
Baseline characteristics in 12-week-old ZDF
rats before chronic peptide administration

| | Scrambled decoy | decoy peptide | P- |
|---|---|---|---|
| | peptide 2 (N = 6) | 2 (N = 6) | value |
| Body weight (g) | 374.2 +/− 24.7 | 385.8 +/− 34.2 | 0.55 |
| Capillary glucose (mg/dL) | 442 +/− 54 | 381 +/− 120 | 0.32 |

Results are mean +/− SD

Capillary glucose concentration was determined by tail nick method (Zimering M B, et al. Endocrinol Diabetes Metab J. 2020; 4(3):413)

Discussion Chronic hypertension is a significant risk factor for the later occurrence of adverse cerebrovascular, cardiovascular and renal outcomes in humans (Mazzaglia, G, et al. Circulation. 2009; 120:1598-1605). Hypertension, diabetes, and obesity each increases the risk of heart failure occurrence in humans (Bui A L, et al. Nat Rev Cardiol. 2011; 8(1):30-41). The global prevalence(s) of aging, obesity and diabetes are expected to increase over the next severals decades thus driving further increase in the global prevalence of obesity-associated hypertension (GBD 2015 Risk Factors Collaborators. Global, regional, and national comparative risk assessment of 79 behavioural, environmental and occupational, and metabolic risks or clusters of risks, 1990-2015: a systematic analysis for the Global Burden of Disease Study 2015. Lancet. 2016 Oct. 8; 388(10053):1659-1724). In 2015, systolic blood pressure was the risk factor accounting for the largest number of global deaths and disability-adjusted life years (GBD 2015 Risk Factors Collaborators. Lancet. 2016 Oct. 8; 388(10053):1659-1724). In a systematic review and meta-analysis of the cardiovascular effect of blood pressure-lowering, every 10 mm Hg reduction in systolic blood pressure was associated with significant reductions in the risks for major cardiovascular events, coronary heart disease, stroke, heart failure and all-cause mortality (Ettehad D, et al. Lancet. 2016; 387(10022):957-967). In high risk patients without diabetes (SPRINT), intensive vs. standard blood pressure-lowering was associated with substantially lower rates of cardiovascular death and all-cause mortality (SPRINT Research Group. Wright J T, Williamson J D. N Engl J Med. 2015; 373:2103-2116). Against this background, the results showing that a single intraperitoneal dose of pegylated decoy peptide 2 (1 mg/kg) caused mean −8.9 mm Hg systolic and mean −9.8 mm Hg diastolic blood pressure-lowering effect compared to (2 mg/kg) IP dose of the scrambled decoy peptide 2 is significant. Remarkably, significant blood pressure-lowering effects were sustained for 30 hours or longer in the male ZDF rats following a single (1 mg/kg) dose of pegylated decoy peptide 2.

Long-term adherence to anti-hypertensive medication regimen is important in reducing cardiovascular event occurrence and mortality risk (The Japanese Society of Hypertension Guidelines for the Management of Hypertension (JSH 2009). Hypertens Res 2009; 32: 3-107). Yet few once-daily, long-acting anti-hypertensive medications achieved sustain substantial blood pressure-lowering effects over 24 hours. In a study comparing valsartan and long-acting amlodipine, four-fold escalation of the initial starting dose of each medication, i.e., valsartan (40 to 160 mg) and amlodipine (2.5 to 10 mg) was undertaken. Even then, once-daily long-acting amlodipine (10 mg) caused a 10% reduction in systolic blood pressure and once-daily valsartan (160 mg) had no significant systolic blood pressure-lowering effect after 24 hours (Eguchi K, et al. Am J Hypertens. 2004; 17(2):112). Single IP administration of (2 mg/kg) decoy peptide 2 caused 19-27% reductions in systolic and diastolic blood pressure after 24 hours; and (1 mg/kg) pepgylated decoy peptide 2 caused 25-26% sustained reductions in systolic and diastolic blood pressure 30 hours after administration in the male ZDF rat. Sustained, substantial blood pressure-lowering by decoy peptide 2 may lead to improved patient adherence, and perhaps be useful in the treatment of acute severe hypertension (in the emergency room) by obviating the need for costly hospitalization.

Pegylated decoy peptide 2 was better tolerated than free decoy peptide 2, i.e., no sedation or hypotension was observed after the initial dose. It's not clear whether the ‘first-dose’ phenomenon observed in a small number of 25-week-old male ZDF rats following IP administration of 2 mg/kg decoy peptide 2 was dose-related or due (in part) to reduced physiological adaptation of aging.

The observation that ten weeks' alternate daily chronic treatment with decoy peptide 2 (2 mg/kg) vs scrambled decoy peptide 2 (2 mg/kg) resulted in significantly lower heart-to-body weight ratio (in male ZDF rats matched for diabetes and obesity) suggesting decoy peptide 2 may be cardioprotective against the deleterious effects of chronic hypertension.

The mechanism of action of decoy peptide 2 is unknown. It is likely that decoy peptide 2 compete with endogenous 5-HT2AR ligands such as 5-HT2A receptor targeting autoantibodies for binding to 5-HT2AR expressed on cells involved in mediating hypertension in the Zucker diabetic fatty rat. This mechanism of action is supported by the finding that exposure of neuroblastoma cells to active, human diabetic 5-HT2AR targeting autoantibodies results in significant downregulation of mRNA expression in the G protein-coupled receptor kinase 3 gene (GRK3) G-protein receptor kinases are a family of serine-threonine kinases which are activated by certain ligand occupied G-protein coupled receptors causing phosphorylation of the GPCR and directing arrestin-mediated receptor desensitization (Eckhart A D, et al. Circ Res. 2000; 86(1):43-50). G-protein receptor kinase 3 has in vivo substrate specificity for ligand occupied-alpha-1B adrenergic receptor (Eckhart A D, et al. Circ Res. 2000; 86(1):43-50), for example, mediating norepinephrine-induced blood pressure elevation or the ionotropic effect of epinephrine and norepinephrine in cardiac muscle. In a recent study (Eckhart A D, et al. Circ Res. 2000; 86(1):43-50), transgenic mice harboring a cardiac specific, constitutively-active mutant alpha1b adrenergic receptor developed cardiac hypertrophy which could be prevented by simultaneous cardiac overexpression of GRK3.

Taken together, these data evidence that decoy peptide 2 may interfere with the downregulation of GRK mRNA expression by binding to 5-HT2AR autoantibodies, thereby restoring normal level of GRK3 mRNA expression in vascular tissues expressing both the 5-HT2AR and the alpha-1 adrenergic receptor. Of interest, GRK3 mRNA expression in human lymphocytes was reported to be inversely associated with systolic and diastolic blood pressure (The Japanese Society of Hypertension Guidelines for the Management of Hypertension (JSH 2009). Hypertens Res 2009; 32: 3-107) perhaps consistent with a regulatory role for GRK3 gene expression in the maintenance of normal blood pressure in humans.

In summary, the decoy peptide 2 caused acute and long-lasting significant systolic and diastolic blood pressure-lowering effects in the Zucker hypertensive diabetic fatty rat. No untoward long-term side effects were observed after chronic (10 weeks) administration, and the results evidence that the chronic blood pressure-lowering effect may be cardioprotective.

### Example 4: Longer-Lasting Blood Pressure Lowering by Myr-SCLLADDN (SEQ ID NO: 9)

Materials and Methods. Animals. Male Zucker diabetic fatty rats (ZDF). Male ZDF Zucker rats were obtained from Charles River Laboratories (Kingston, NY) at approximately 6-7 weeks of age. The rats were single housed upon arrival, without enrichment. Rats were acclimatized for two weeks prior to experimental procedures. Rats were provided ad libitum access to food and water and maintained in a 12 h light/dark cycle with lights on at 0630. All procedures occurred during the light phase of the cycle.

Synthetic peptides. The peptides were synthesized at Lifetein Inc. (Hillborough, NJ). The lyophilized peptides were aliquoted and stored (in the presence of desiccant) at −40° C. prior to use. On the day of intraperitoneal (IP) administration, an aliquot of lyophilized unconjugated peptide was reconstituted in sterile saline at the appropriate concentration of 2 mg/kg. Reconstituted peptide was prepared fresh before each injection. Because of its lower solubility in water, myristic acid-conjugated peptide was first reconstituted in a small volume of DMSO, prior to addition of sterile saline. The final concentration of DMSO in sterile saline was 10%. The vehicle, DMSO in sterile saline, was tested independently and had no effect on blood pressure when administered as an IP injection.

A linear synthetic peptide, SCLLADDN (SEQ ID NO: 2) was synthesized and had ≥95% purity.

Myristic acid-conjugated to the decoy peptide. Myristic acid was conjugated to SCLLADDN (SEQ ID NO: 2) on the free N-terminus, Myr-SCLLADDN (SEQ ID NO: 9). It had purity of 91.7% and MW 1040.

Blood pressure monitoring. Tail cuff blood pressure measurement was performed using an automated CODA noninvasive blood pressure system (Kent Scientific, Torrington, CT.). Rats were placed on an insulated warming platform in a well-heated room to ensure proper body temperature. Cuff monitors were provided in different sizes to accommodate rats of different ages and having different body weights. After the animal was positioned in the clear plastic restraint holder, the appropriate-sized cuff was slid over the rat's tail. The blood pressure system uses volume pressure recording tail-cuff technology and displays up to six blood pressure measurements per cycle.

Experiment 1: Four 12-week-old male ZDF rats were randomly assigned to receive either a single IP injection of a scrambled sequence (SEQ ID NO: 7) of Peptide ID #2 (SEQ ID NO: 2) at 1 mg/kg concentration or a 1 mg/kg concentration of myristolated Peptide ID #2 (SEQ ID NO: 9). Blood pressure was determined using tail cuff plethysmography (Kent Scientific) at 1, 4, 6, 48 and 72 hours post-injection.

Experiment 2: Six 12-week-old male ZDF rats were assigned to each of three treatment groups (N=2/group). The first group received a 1 mg/kg IP injection of a scrambled peptide (SEQ ID NO: 7). The second group received a 1 mg/kg IP injection of myristolated Peptide ID #2 (SEQ ID NO: 9), and the third group received a 1.5 mg/kg IP injection of myristolated Peptide ID #2 (SEQ ID NO: 9). Blood pressure was determined at 4, 24, 48, 72, 96 and 168 hours after single IP injection.

Experiment 3: Six 13-week-old male ZDF rats were assigned to three treatment groups (N=2/group). The first group received a 1 mg/kg IP injection of a scrambled peptide (SEQ ID NO: 7). The second group received a 2 mg/kg IP injection of myristolated Peptide ID #2 (SEQ ID NO: 9), and the third group received a 2.5 mg/kg IP injection of myristolated Peptide ID #2 (SEQ ID NO: 9). Blood pressure was determined at 4, 24, 48, 72, and 144 hours after single IP injection.

Results. A 1 mg/kg concentration of myristolated Peptide ID 2 (SEQ ID NO: 9) (delivered via intraperitoneal injection) caused rapid-onset diastolic blood pressure lowering in male ZDF rats (N=2) one hour following administration. Significant diastolic blood pressure lowering-effect appeared to persist for between 48 to 72 hours or longer after a single injection. No discernable adverse physical or behavioral effects following drug administration was observed. Scrambled peptide (1 mg/kg IP; SEQ ID NO: 7) (N=2) did not cause significant diastolic blood pressure (DBP) lowering at any post-baseline timepoint compared to the baseline diastolic blood pressure. Mean arterial blood pressure (MAP) was significantly reduced 1-72 hours following a single IP injection of 1 mg/kg myristolated Peptide ID 2 (SEQ ID NO: 9). Scrambled peptide (1 mg/kg single IP injection; SEQ ID NO: 7)) did not cause significant mean arterial blood pressure-lowering over the same follow up time period. Systolic blood pressure (SBP) was significantly lowered after a single 1 mg/kg IP injection of myristolated Peptide ID 2 (SEQ ID NO: 9), and was also significantly lower at 1, 4, 6 hours and 48 hours post-baseline, but not at 72 hours compared to its baseline level.

Table 10 shows the results of two groups of male Zucker diabetic fatty rats (11-12 weeks-old; n=4 per group) that received either a single IP dose of Myr-Peptide ID 2 (2 mg/kg; SEQ ID NO: 9) or IP scrambled Peptide 2 (1 mg/kg; SEQ ID NO: 7). Blood pressure was determined by tail cuff plethysmography at regular daily intervals for up to six days after drug injection. Time series statistical analysis was used to test for a statistically significant difference in the overall systolic and/or diastolic blood pressure lowering effect of Myr-Peptide 2 vs scrambled peptide over the entire 6-day duration following injection.

TABLE 10
Comparison of diastolic (A) and systolic (B) blood pressure
lowering effect after administration of single IP dose of
Myr-peptide 2 (SEQ ID NO: 9) vs scrambled peptide 2 (SEQ
ID NO: 7) in two groups of 11-week-old male Zucker diabetic,
fatty rats (N = 4 rats per drug treatment group).

| | | | | | |
|---|---|---|---|---|---|
| | | Standard | | | |
| Drug | Estimate | Error | DF | t Value | Pr > \|t\| |

A)

| | | | | | |
|---|---|---|---|---|---|
| Myr-Peptide 2 | −12.0471 | 3.2320 | 5 | −3.73 | 0.0136 |
| Scrambled Peptide | 0 | . | . | . | . |

B)

| | | | | | |
|---|---|---|---|---|---|
| Myr-Peptide 2 | −17.4393 | 5.0163 | 5 | −3.48 | 0.0177 |
| Scrambled Peptide | 0 | . | . | . | . |

Table 10 shows that a single 2 mg/kg IP dose of Myr-Peptide ID 2 (SEQ ID NO: 9) caused a statistically significant 12 mm Hg mean lowering in diastolic blood pressure (Table 10A) and a statistically significant 17.4 mm Hg mean lowering in systolic blood pressure (Table 10B) compared to 1 mg/kg scrambled Peptide 2 (P<0.02; SEQ ID NO: 9). No significant effect of time on blood pressure-lowering effect from the two drugs was observed. Because baseline mean systolic and diastolic blood pressure were different in the two groups of rats that received either Myr-Peptide ID 2 (SEQ ID NO: 9) or scrambled Peptide 2 (SEQ ID NO: 9), the model included an adjustment for difference in baseline mean blood pressure.

Experiment 2. A second group of 12-week-old male Zucker diabetic fatty rats (N=6) was randomly assigned to receive either a single IP administration of a scrambled peptide (1 mg/kg; N=2; SEQ ID NO: 7), or a single IP administration of a 1 mg/kg concentration of myristolated Peptide ID 2 (N=2; SEQ ID NO: 9) or a 1.5 mg/kg concentration of myristolated peptide ID 2 (N=2; SEQ ID NO: 9). Injections were administered via the intraperitoneal IP route. Blood pressure was determined before and at 4, 24, 48, 72, 96 and 168 hours after a single IP drug administration.

Scrambled peptide (1 mg/kg; N=2; SEQ ID NO: 7) did not cause a significant change in systolic blood pressure at any post-baseline time point compared to its baseline level. In two male ZDF rats who each received a 1 mg/kg dose, myristolated Peptide ID #2 (SEQ ID NO: 9) caused significant systolic blood pressure-lowering (e.g., by 8-11% compared to baseline) at 4 hours post-administration which persisted for 48-72 hours in one rat or for 96-168 hours in the other rat.

In two male ZDF rats who each received a 1.5 mg/kg dose of the myristolated Peptide ID #2 (SEQ ID NO: 9) caused significant systolic blood pressure-lowering at 4 hours post-administration (e.g., by 13% compared to baseline) which persisted for up to 168 hours (7 days) following drug administration. Changes in diastolic or mean arterial blood pressure in ZDF rats who received a single IP injection of either scrambled peptide (SEQ ID NO: 7) or myristolated Peptide ID2 (SEQ ID NO: 9) mirrored changes observed for systolic blood pressure. That is, a 1 mg/kg dose of myristolated Peptide ID2 (SEQ ID NO: 9) caused significant declines in diastolic (10-12%) and mean arterial blood pressure (6-8%) vs baseline that persisted for either 2-3 days (rat 1) or 4-7 days (rat 2). A higher dose (1.5 mg/kg) of myristolated Peptide ID2 (SEQ ID NO: 9) caused a longer-lasting decline in both diastolic (11-19%) and mean arterial blood pressure (14%) vs baseline in both rats tested compared to the 1.0 mg/kg dose (10-12%; 6-8%, respectively). Blood pressure-lowering appeared to persist for up to 7 days in both rats who received the 1.5 mg/kg dose of myristolated Peptide 2 (SEQ ID NO: 9). None of the ten rats tested (N=4 in Expt 1; N=6 in Expt 2) experienced any untoward physical or behavioral effect for up to 14-21 days after single IP administration of either myristolated Peptide ID2 (SEQ ID NO: 9) or a scrambled sequence of Peptide ID 2 (SEQ ID NO: 7).

Experiment 3. Acute changes in blood pressure around the time of decoy peptide administration. Experiment 3 was designed to evaluate blood pressure changes to higher doses of myristolated Peptide 2 (2.0 mg/kg and 2.5 mg/kg; SEQ ID NO: 9) administered as a single IP injection in male ZDF (N=2 rats per dose); and a simultaneous control group (two age-matched 13-week old male ZDF rats) that received a single IP dose of 1 mg/kg scrambled peptide (SEQ ID NO: 7). The results show a 20-23% peak acute drop in systolic blood pressure in response to single IP dose of 2.5 mg/kg myristolated peptide ID 2 (SEQ ID NO: 9); P<0.01 compared to baseline (time zero) systolic blood pressure). Thirteen-week-old male Zucker diabetic fatty rats tested were naïve to anti-hypertensive medication exposure. The results also show a 18-35% acute peak drop in diastolic blood pressure from 2.5 mg/kg Myr Peptide 2 (SEQ ID NO: 9; P<0.01 compared to baseline (time zero) systolic blood pressure.

Two 13-week-old male Zucker diabetic fatty rats naïve to anti-hypertensive medication exposure). A 28-30% acute peak drop in mean arterial blood pressure was also observed in response to 2.5 mg/kg Myr Peptide 2 (SEQ ID NO: 9; P<0.01 compared to baseline (time zero) mean arterial blood pressure. Two 13-week-old male Zucker diabetic fatty rats naïve to anti-hypertensive medication exposure). A 7-15% acute drop in systolic blood pressure in response to single IP dose of 2 mg/kg myristolated peptide ID 2 (SEQ ID NO: 2; P<0.01 compared to baseline; A P<0.05 vs 4 hr timepoint. Two thirteen-week-old male ZDF rats naïve to prior anti-hypertensive medication exposure) was observed. A 22-25% acute drop in diastolic blood pressure was observed in response to a single IP dose of 2 mg/kg myristolated peptide ID 2 (SEQ ID NO: 9; P<0.01 compared to baseline; A P<0.05 vs 4 hr timepoint). Further, a 14-22% acute peak drop in mean arterial blood pressure in response to 2 mg/kg Myr Peptide 2 (SEQ ID NO: 9; P<0.01 compared to baseline; A P<vs 4 hr timepoint. Thirteen-week-old male ZDF rats naïve to prior anti-hypertensive medication exposure) was observed. As summarized in Tables 11-13, acute reductions in systolic, diastolic and mean arterial blood pressure were quite substantial and dose-dependent within the first 24 hours after single IP dose administration. Scrambled peptide (1 mg/kg; SEQ ID NO: 7) had no significant acute or chronic blood pressure-lowering effect compared to baseline level in two ZDF rats.

TABLE 11
Dose-dependence of acute and durability of diastolic
blood pressure-lowering effects of single IP administration
of myristolated Peptide ID #2 (SEQ ID NO: 9).

| | | |
|---|---|---|
| Myristolated | Acute Peak | Duration of |
| Peptide 2 Dose | blood pressure-lowering (%) | Action (days) |

Diastolic BP

| | | | |
|---|---|---|---|
| 1 | mg/kg | 10-12% | 4-6 |
| 1.5 | mg/kg | 19% | 7 |
| 2.0 | mg/kg | 20-25%. | 3; 7 |
| 2.5 | mg/kg | 18-36% | 9 |

Results are based on treatment of two male ZDF rats at each dose.

TABLE 12
Dose-dependence of acute and durability of mean arterial
blood pressure-lowering effects of single IP administration
of myristolated Peptide ID #2 (SEQ ID NO: 9).

| | | |
|---|---|---|
| Myristolated | Acute Peak | Duration of |
| Peptide 2 Dose | blood pressure-lowering (%) | Action (days) |

Mean arterial BP

| | | | |
|---|---|---|---|
| 1 | mg/kg | 6-8% | 4-6 |
| 1.5 | mg/kg | 14% | 7 |
| 2.0 | mg/kg | 14-22% | 3; 7 |
| 2.5 | mg/kg | 19-30% | 9 |

Results are based on treatment of two male ZDF rats at each dose

TABLE 13
Dose-dependence of acute and durability of systolic
blood pressure-lowering effects of single IP administration
of myristolated Peptide ID #2 (SEQ ID NO: 9).

| | | |
|---|---|---|
| Myristolated | Acute Peak | Duration of |
| Peptide 2 Dose | blood pressure-lowering (%) | Action (days) |

Systolic BP

| | | | |
|---|---|---|---|
| 1 | mg/kg | 4-10% | 4-7; 7 |
| 1.5 | mg/kg | 10% | 7 |
| 2.0 | mg/kg | 15% | 2-3; 6 |
| 2.5 | mg/kg | 23% | 9 |

Results are based on treatment of two male ZDF rats at each dose.

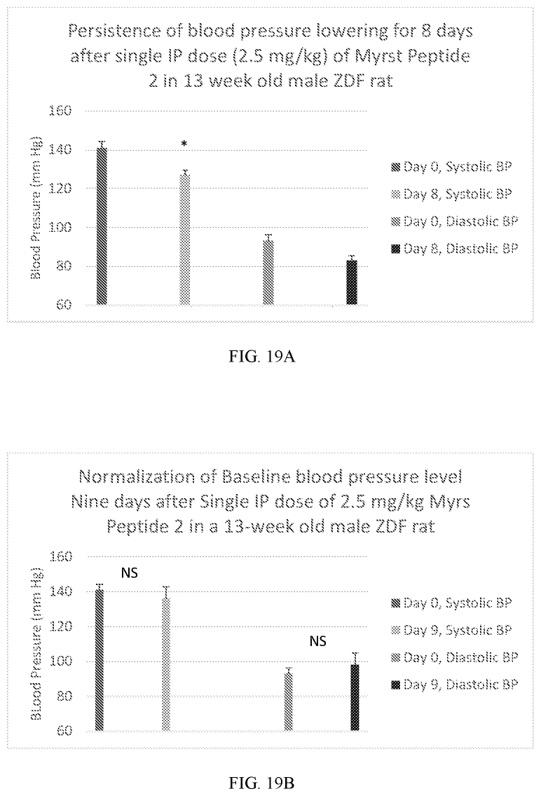

Long-lasting changes in blood pressure following single IP drug administration of SEQ ID NO: 9). Blood pressure was monitored for 7-9 days after single IP administration of a myristolated decoy peptide (e.g., SEQ ID NO: 9). Following normalization of blood pressure to its baseline pre-treatment level, daily blood pressure monitoring was stopped. However, because two of two male ZDF rats treated with the highest dose of myristolated peptide (2.5 mg/kg; SEQ ID NO: 9) both continued to exhibit lower blood pressure compared to pre-treatment level even up to 7 days after drug administration, these two rats were monitored for total of 9 days after single peptide dosing. The blood pressure results (on day 8 and 9) in these two rats treated with 2.5 mg/kg myristolated peptide 2 are shown separately in FIGS. 19A-B.

A single IP injection of 1 mg/kg dose of scrambled peptide did not significantly reduce systolic, diastolic or mean arterial blood pressure at 4 hours, 48, 72, 144 or 168 hours compared to baseline blood pressure. The duration of significant blood pressure-lowering following a single IP dose of 2.5 mg/kg Myristolated Peptide 2 (SEQ ID NO: 9) was unexpectedly long. Seven and/or eight days following IP administration of the maximal dose tested, persistence of significant systolic and diastolic blood pressure-lowering compared to their respective baseline levels (FIG. 19A) was observed. It was not until 9 days following a single IP dose of 2.5 mg/kg myristolated peptide (SEQ ID NO: 9) administration that the systolic and diastolic blood pressure returned to their respective baseline, pre-treatment levels (FIG. 19B). Similar results were observed in two, 13-week old male ZDF rats.

Taken together, these data suggest that myristolation of decoy peptide 2 (e.g., SEQ ID NO: 9) substantially extends the in vivo pharmacodynamic duration of blood pressure-lowering to 9 days following a single IP administration of the maximal dose of myristolated decoy Peptide 2 (SEQ ID NO: 9) tested. Differences in the duration of blood pressure-lowering effects were in part dose-dependent Conjugation to a fatty acid can enhance lipid solubility. Injection into the subcutaneous fat depot may result in slow release of the myristolated decoy peptide into the circulation prolonging the duration of action, especially when supra-physiologic doses (2.5 mg/kg) were injected. The blood pressure-lowering effects observed in response to a single IP dose(s) ranging from 1.0-2.5 mg/kg of Myr Peptide 2 was potent, dose-dependent, and in many cases unexpectedly quite long-lasting.

### Example 5: Longer-Lasting Blood Pressure Lowering by Myr-SCLLADDN (SEQ ID NO: 9) Compared to Unconjugated Peptide ID 2 (SEQ ID NO: 2)

Materials and Methods. Animals. Male Zucker diabetic fatty rats (ZDF). Male ZDF Zucker rats were obtained from Charles River Laboratories (Kingston, NY) at approximately 6-7 weeks of age. Rats were single housed upon arrival, without enrichment. Rats were acclimatized for two weeks prior to experimental procedures. Rats were provided ad libitum access to food and water and maintained in a 12 h light/dark cycle with lights on at 0630. All procedures occurred during the light phase of the cycle.

Synthetic peptides. Peptides were synthesized at Lifetein Inc. (Hillborough, NJ). The lyophilized peptides were aliquoted and stored (in the presence of desiccant) at −40° C. prior to use. On the day of intraperitoneal (IP) administration, an aliquot of lyophilized unconjugated peptide was reconstituted in sterile saline at the appropriate concentration of 2 mg/kg. Reconstituted peptide was prepared fresh before each injection. Because of its lower solubility in water, myristic acid-conjugated peptide was first reconstituted in a small volume of DMSO, prior to addition of sterile saline. The final concentration of DMSO in sterile saline was 10%. The vehicle, DMSO in sterile saline, was tested independently and had no effect on blood pressure when administered as an IP injection.

Decoy receptor peptide. A linear synthetic peptide, SCLLADDN (SEQ ID NO: 2) was synthesized and had ≥95% purity.

Myristic acid-conjugated SCLLADDN (SEQ ID NO: 9). Myristic acid was conjugated to SCLLADDN (SEQ ID NO: 2) on the free N-terminus, Myr-SCLLADDN (SEQ ID NO: 9). It had purity of 91.7% and MW 1040.

Blood pressure monitoring. Tail cuff blood pressure measurement was performed using an automated CODA noninvasive blood pressure system (Kent Scientific, Torrington, CT.). Rats were placed on an insulated warming platform in a well-heated room to ensure proper body temperature. Cuff monitors were provided in different sizes to accommodate rats of different ages and having different body weights. After the animal was positioned in the clear plastic restraint holder, the appropriate-sized cuff was slid over the rat's tail. The blood pressure system uses volume pressure recording tail-cuff technology and displays up to six blood pressure measurements per cycle.

Results. Myristic acid conjugated Peptide ID 2 (SEQ ID NO: 9) had significantly longer duration of blood pressure lowering (6 days or longer) after single IP injection of a 2 mg/kg dose compared to identical concentration of unconjugated Peptide ID 2 (24-48 hours; SCLLADDN (SEQ ID NO: 2). Tables 13 and 14 shows the comparison of the duration of blood-pressure-lowering effects of SEQ ID NO: 2 (unconjugated decoy peptide) to SEQ ID NO: 9 (myristic acid conjugated to decoy peptide 2). This finding demonstrates that myristic acid conjugated to a decoy peptide (e.g., SEQ ID NO: 9) significantly lowers blood pressure and can be self-administered once a week, improving patient compliance to long-term anti-hypertensive use. The unexpectedly long duration of action of myristic acid-conjugated SCLLADDN (SEQ ID NO: 9) is likely due to the fact that myristic acid is hydrophobic and tends to be membrane associated, both with the plasma membrane and the inner leaflet of the cell membrane. It is likely that following IP injection of conjugated (myristic acid) Peptide ID 2 in the abdominal fat depot of the male ZDF rat, that there is slower release into the general circulation compared to unconjugated Peptide ID 2, and more importantly, once myristic acid-conjugated Peptide ID 2 gains access to the circulation it likely partitions inside the cell in association with plasma membrane which greatly slows its enzymatic degradation in the proximal renal tubular cells of the kidney—the site of most small peptide degradation.

Comparison of duration of blood pressure-lowering effect from single 2 mg/kg IP dose of unconjugated (Table 14) vs. myristolated (Table 15) Peptide ID 2

TABLE 14
UNCONJUGATED PEPTIDE ID 2 (SEQ ID NO: 2)
Blood pressure (BP)

| | |
|---|---|
| Baseline | 130/96 |
| 6 hours | 109/71\hat{ } |
| 24 hours | 105/73**,\hat{ } |
| 48 hours | 137/93 |

Results are mean systolic/diastolic BP in two male 25-week-old Zucker diabetic fatty rats that each received a single 2 mg/kg IP dose of unconjugated Peptide ID 2 (SEQ ID NO: 2)
\hat{ }P < 0.05 - comparing postbaseline diastolic BP to baseline diastolic bp
*** P < 0.001- comparing postbaseline (24 hr) systolic BP to baseline systolic bp

TABLE 15
MYRISTOLATED PEPTIDE ID 2 (SEQ ID NO: 9)
Blood pressure

| | |
|---|---|
| Baseline. | 146/97 |
| 24 hours. | 117/76 |
| 48 hours | 123/82 |
| 72 hours | 126/80 **, \hat{ } |
| 6 days | 127/85 ** |
| 7 days | 142/94 |

Results are mean systolic/diastolic BP in four male 11-12-week-old Zucker diabetic fatty rats that each received a single 2 mg/kg IP dose of myristolated Peptide ID 2 (SEQ ID NO: 9)
\hat{ } P < 0.05 - comparing postbaseline (72 hr) diastolic BP to baseline diastolic bp
** P ≤ 0.01- comparing postbaseline (72 hr or 6 days) systolic BP to baseline systolic bp

1.-69. (canceled)

70. A decoy peptide consisting of the sequence QDDSKVFKEGSCLLADDN (SEQ ID NO: 1), or a fragment thereof, wherein the fragment of SEQ ID NO: 1 is 4 to 9 amino acids in length, wherein the decoy peptide comprises a fatty acid attached to the N-terminus.

71. The decoy peptide of claim 70, wherein the decoy peptide inhibits the binding of 5-HT2A receptor autoantibodies to a second extracellular loop region of the 5-HT2A receptor.

72. The decoy peptide of claim 70, wherein the decoy peptide consists of the sequence SCLLADDN (SEQ ID NO: 2), QDDSKVF (SEQ ID NO: 3), or VFKEGSC (SEQ ID NO: 4).

73. The decoy peptide of claim 70, wherein the decoy peptide further comprises at least one polyethylene glycolated (PEGylated) group connecting the fatty acid to the decoy peptide.

74. The decoy peptide of claim 70, wherein the fatty acid is butyric acid, caproic acid, caprylic acid, capric acid, decanoic acid, lauric acid, myristic acid, palmitic acid, pentadecanoic acid, stearic acid, arachidic acid, behenic acid, erucic acid, lignoceric acid, margaric acid, myristoleic acid, palmitoleic acid, oleic acid, gadoleic acid, ricinoleic acid, vaccenic acid, linoleic acid, linolenic acid, alpha-linolenic acid, gamma-linolenic acid, licanic acid, margaroleic acid, arachidic acid, gadoleic acid, nervonic acid, arachidonic acid, docosapentaenoic (DPA), eicosapentaenoic acid (EPA), or docosahexaenoic acid (DHA).

75. The decoy peptide of claim 70, wherein at least one amino acid residue of the decoy peptide comprises an acetyl group, a fluorenylmethoxy carbonyl group, a formyl group, a palmitoyl group, a myristyl group, a stearyl group, or polyethylene glycol.

76. A pharmaceutical composition comprising the decoy peptide of claim 70, and a pharmaceutically acceptable carrier.

77. A method of lowering plasma triglycerides or alanine transaminase (ALT) levels in a subject, the method comprising: administering to the subject a therapeutically effective amount of a decoy peptide, wherein the decoy peptide comprises or consists of the sequence QDDSKVFKEGSCLLADDN (SEQ ID NO: 1), or a fragment thereof, and a pharmaceutically acceptable carrier.

78. The method of claim 77, wherein the subject has or has been diagnosed with type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia or has had a stroke or a traumatic brain injury or a combination thereof.

79. The method of claim 77, wherein the subject's body weight change is lowered as compared to the subject's body weight change without administration of the therapeutically effective amount of the decoy peptide.

80. The method of claim 77, wherein plasma levels of aspartate aminotransferase (AST) in the subject decrease as compared to plasma AST levels prior to administering the therapeutically effective amount of the decoy peptide.

81. The method of claim 77, wherein the decoy peptide consists of the sequence SCLLADDN (SEQ ID NO: 2).

82. A method of reducing or preventing cardiac hypertrophy in a subject, the method comprising: administering to the subject a therapeutically effective amount of a decoy peptide, wherein the decoy peptide comprises or consists of the sequence QDDSKVFKEGSCLLADDN (SEQ ID NO: 1), or a fragment thereof, and a pharmaceutically acceptable carrier.

83. The method of claim 82, wherein the subject has or has been diagnosed with type 2 diabetes, a neurologic disease or disorder, a microvascular disease or disorder, hypertension, obesity, microvascular angiopathy, Parkinson's disease, dementia, major depressive disorder, schizophrenia, retinitis pigmentosa, refractory hypertension, mild cognitive dysfunction, primary open angle glaucoma, diabetic dyslipidemia, hypertriglyceridemia, type 2B hyperlipidemia or has had a stroke or a traumatic brain injury or a combination thereof.

84. The method of claim 82, wherein the decoy peptide consists of the sequence SCLLADDN (SEQ ID NO: 2).

85. A method of identifying a subject having or at risk of developing hypertriglyceridemia or having or at risk of developing elevated levels of alanine aminotransferase (ALT), the method comprising:
a) obtaining or having obtained a sample from the subject, wherein the sample comprises one or more 5-HT2A receptor autoantibodies;
b) contacting the sample of step a) with one or more decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4);
c) determining the level of binding of the 5-HT2A receptor autoantibodies to one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4); and
d) identifying the subject having or at risk for developing hypertriglyceridemia when the level of binding in step c) is higher when compared to the level of binding in a reference sample or identifying the subject having or at risk of developing elevated levels of ALT when the level of binding in step c) is higher when compared to the level of binding in a reference sample, respectively.

86. The method of claim 85, wherein the subject has type 2 diabetes, refractory hypertension, essential hypertension or obesity.

87. The method of claim 85, further comprising administering to the subject a therapeutically effective amount of a pharmaceutical composition comprising a decoy peptide comprising the sequence of SEQ ID NO: 2, and a pharmaceutically acceptable carrier.

88. The method of claim 85, wherein the level of binding of 5-HT2A receptor autoantibodies to one or more of the decoy peptides comprising the sequence of QDDSKVFKEGSCLLADDN (SEQ ID NO: 1), SCLLADDN (SEQ ID NO: 2), QDDSKVF (SEQ ID NO: 3), or VFKEGSC (SEQ ID NO: 4) is determined by ELISA.

89. The method of claim 85, further comprising administering a therapeutically effective amount of ketanserin or volinanserin to the subject.


SEQUENCE LISTING (105 sequences)

SEQ ID NO: 1 - protein, 18 aa, Artificial Sequence
  Synthetic construct
QDDSKVFKEGSCLLADDN

SEQ ID NO: 2 - protein, 8 aa, Artificial Sequence
  Synthetic construct
SCLLADDN

SEQ ID NO: 3 - protein, 7 aa, Artificial Sequence
  Synthetic construct
QDDSKVF

SEQ ID NO: 4 - protein, 7 aa, Artificial Sequence
  Synthetic construct
VFKEGSC

SEQ ID NO: 5 - protein, 8 aa, Artificial Sequence
  Synthetic construct
  MOD_RES (1)..(1): Lys(Azide)
SCLLADDN

SEQ ID NO: 6 - protein, 8 aa, Artificial Sequence
  Synthetic construct
  MOD_RES (1)..(1): K(DBCO-DSPE-PEG2K)
SCLLADDN

SEQ ID NO: 7 - protein, 8 aa, Artificial Sequence
  Synthetic construct
LASNDCLD

SEQ ID NO: 8 - protein, 4 aa, Artificial Sequence
  Synthetic construct
  MISC_FEATURE (2)..(2): S or T
  MISC_FEATURE (3)..(3): any amino acid
  MISC_FEATURE (4)..(4): V or I
EXXX

SEQ ID NO: 9 - protein, 8 aa, Artificial Sequence
  Synthetic construct
  MOD_RES (1)..(1): Myristic acid
SCLLADDN

SEQ ID NO: 10 - protein, 18 aa, Artificial Sequence
  Synthetic construct
  MOD_RES (1)..(1): butanoyl
QDDSKVFKEGSCLLADDN

SEQ ID NO: 11 - protein, 18 aa, Artificial Sequence
  Synthetic construct
  MOD_RES (1)..(1): hexanoyl
QDDSKVFKEGSCLLADDN

SEQ ID NO: 12 - protein, 18 aa, Artificial Sequence
  Synthetic construct
  MOD_RES (1)..(1): octanoyl
QDDSKVFKEGSCLLADDN

SEQ ID NO: 13 - protein, 18 aa, Artificial Sequence
  Synthetic construct
  MOD_RES (1)..(1): decanoyl
QDDSKVFKEGSCLLADDN

SEQ ID NO: 14 - protein, 18 aa, Artificial Sequence
  Synthetic construct
  MOD_RES (1)..(1): lauroyl
QDDSKVFKEGSCLLADDN

SEQ ID NO: 15 - protein, 18 aa, Artificial Sequence
  Synthetic construct
  MOD_RES (1)..(1): myristoyl
QDDSKVFKEGSCLLADDN

SEQ ID NO: 16 - protein, 18 aa, Artificial Sequence
  Synthetic construct
  MOD_RES (1)..(1): palmitoyl
QDDSKVFKEGSCLLADDN

SEQ ID NO: 17 - protein, 18 aa, Artificial Sequence
  Synthetic construct
  MOD_RES (1)..(1): stearoyl
QDDSKVFKEGSCLLADDN

SEQ ID NO: 18 - protein, 18 aa, Artificial Sequence
  Synthetic construct
  MOD_RES (1)..(1): palmitoleoyl
QDDSKVFKEGSCLLADDN

SEQ ID NO: 19 - protein, 18 aa, Artificial Sequence
  Synthetic construct
  MOD_RES (1)..(1): arachidoyl
QDDSKVFKEGSCLLADDN

SEQ ID NO: 20 - protein, 18 aa, Artificial Sequence
  Synthetic construct
  MOD_RES (1)..(1): behenoyl
QDDSKVFKEGSCLLADDN

SEQ ID NO: 21 - protein, 18 aa, Artificial Sequence
  Synthetic construct
  MOD_RES (1)..(1): oleoyl
QDDSKVFKEGSCLLADDN

SEQ ID NO: 22 - protein, 18 aa, Artificial Sequence
  Synthetic construct
  MOD_RES (1)..(1): ricinoleoyl
QDDSKVFKEGSCLLADDN

SEQ ID NO: 23 - protein, 18 aa, Artificial Sequence
  Synthetic construct
  MOD_RES (1)..(1): linolenoyl
QDDSKVFKEGSCLLADDN

SEQ ID NO: 24 - protein, 18 aa, Artificial Sequence
  Synthetic construct
  MOD_RES (1)..(1): vacceoyl
QDDSKVFKEGSCLLADDN

SEQ ID NO: 25 - protein, 18 aa, Artificial Sequence
  Synthetic construct
  MOD_RES (1)..(1): gadoleoyl
QDDSKVFKEGSCLLADDN

SEQ ID NO: 26 - protein, 18 aa, Artificial Sequence
  Synthetic construct
  MOD_RES (1)..(1): erucoyl
QDDSKVFKEGSCLLADDN

SEQ ID NO: 27 - protein, 18 aa, Artificial Sequence
  Synthetic construct
  MOD_RES (1)..(1): cetoleoyl
QDDSKVFKEGSCLLADDN

SEQ ID NO: 28 - protein, 18 aa, Artificial Sequence
  Synthetic construct
  MOD_RES (1)..(1): nervonoyl
QDDSKVFKEGSCLLADDN

SEQ ID NO: 29 - protein, 18 aa, Artificial Sequence
  Synthetic construct
  MOD_RES (1)..(1): adrenoyl
QDDSKVFKEGSCLLADDN

SEQ ID NO: 30 - protein, 18 aa, Artificial Sequence
  Synthetic construct
  MOD_RES (1)..(1): alpha-linolenoyl
QDDSKVFKEGSCLLADDN

SEQ ID NO: 31 - protein, 18 aa, Artificial Sequence
  Synthetic construct
  MOD_RES (1)..(1): gamma-linolenoyl
QDDSKVFKEGSCLLADDN

SEQ ID NO: 32 - protein, 18 aa, Artificial Sequence
  Synthetic construct
  MOD_RES (1)..(1): EPA
QDDSKVFKEGSCLLADDN

SEQ ID NO: 33 - protein, 18 aa, Artificial Sequence
  Synthetic construct
  MOD_RES (1)..(1): DHA
QDDSKVFKEGSCLLADDN

SEQ ID NO: 34 - protein, 8 aa, Artificial Sequence
  Synthetic construct
  MOD_RES (1)..(1): butanoyl
SCLLADDN

SEQ ID NO: 35 - protein, 8 aa, Artificial Sequence
  Synthetic construct
  MOD_RES (1)..(1): hexanoyl
SCLLADDN

SEQ ID NO: 36 - protein, 8 aa, Artificial Sequence
  Synthetic construct
  MOD_RES (1)..(1): octanoyl
SCLLADDN

SEQ ID NO: 37 - protein, 8 aa, Artificial Sequence
  Synthetic construct
  MOD_RES (1)..(1): decanoyl
SCLLADDN

SEQ ID NO: 38 - protein, 8 aa, Artificial Sequence
  Synthetic construct
  MOD_RES (1)..(1): lauroyl
SCLLADDN

SEQ ID NO: 39 - protein, 8 aa, Artificial Sequence
  Synthetic construct
  MOD_RES (1)..(1): myristoyl
SCLLADDN

SEQ ID NO: 40 - protein, 8 aa, Artificial Sequence
  Synthetic construct
  MOD_RES (1)..(1): palmitoyl
SCLLADDN

SEQ ID NO: 41 - protein, 8 aa, Artificial Sequence
  Synthetic construct
  MOD_RES (1)..(1): stearoyl
SCLLADDN

SEQ ID NO: 42 - protein, 8 aa, Artificial Sequence
  Synthetic construct
  MOD_RES (1)..(1): palmitoleoyl
SCLLADDN

SEQ ID NO: 43 - protein, 8 aa, Artificial Sequence
  Synthetic construct
  MOD_RES (1)..(1): arachidoyl
SCLLADDN

SEQ ID NO: 44 - protein, 8 aa, Artificial Sequence
  Synthetic construct
  MOD_RES (1)..(1): behenoyl
SCLLADDN

SEQ ID NO: 45 - protein, 8 aa, Artificial Sequence
  Synthetic construct
  MOD_RES (1)..(1): oleoyl
SCLLADDN

SEQ ID NO: 46 - protein, 8 aa, Artificial Sequence
  Synthetic construct
  MOD_RES (1)..(1): ricinoleoyl
SCLLADDN

SEQ ID NO: 47 - protein, 8 aa, Artificial Sequence
  Synthetic construct
  MOD_RES (1)..(1): linolenoyl
SCLLADDN

SEQ ID NO: 48 - protein, 8 aa, Artificial Sequence
  Synthetic construct
  MOD_RES (1)..(1): vacceoyl
SCLLADDN

SEQ ID NO: 49 - protein, 8 aa, Artificial Sequence
  Synthetic construct
  MOD_RES (1)..(1): gadoleoyl
SCLLADDN

SEQ ID NO: 50 - protein, 8 aa, Artificial Sequence
  Synthetic construct
  MOD_RES (1)..(1): erucoyl
SCLLADDN

SEQ ID NO: 51 - protein, 8 aa, Artificial Sequence
  Synthetic construct
  MOD_RES (1)..(1): cetoleoyl
SCLLADDN

SEQ ID NO: 52 - protein, 8 aa, Artificial Sequence
  Synthetic construct
  MOD_RES (1)..(1): nervonoyl
SCLLADDN

SEQ ID NO: 53 - protein, 8 aa, Artificial Sequence
  Synthetic construct
  MOD_RES (1)..(1): adrenoyl
SCLLADDN

SEQ ID NO: 54 - protein, 8 aa, Artificial Sequence
  Synthetic construct
  MOD_RES (1)..(1): alpha-linolenoyl
SCLLADDN

SEQ ID NO: 55 - protein, 8 aa, Artificial Sequence
  Synthetic construct
  MOD_RES (1)..(1): gamma-linolenoyl
SCLLADDN

SEQ ID NO: 56 - protein, 8 aa, Artificial Sequence
  Synthetic construct
  MOD_RES (1)..(1): EPA
SCLLADDN

SEQ ID NO: 57 - protein, 8 aa, Artificial Sequence
  Synthetic construct
  MOD_RES (1)..(1): DHA
SCLLADDN

SEQ ID NO: 58 - protein, 7 aa, Artificial Sequence
  Synthetic construct
  MOD_RES (1)..(1): butanoyl
QDDSKVF

SEQ ID NO: 59 - protein, 7 aa, Artificial Sequence
  Synthetic construct
  MOD_RES (1)..(1): hexanoyl
QDDSKVF

SEQ ID NO: 60 - protein, 7 aa, Artificial Sequence
  Synthetic construct
  MOD_RES (1)..(1): octanoyl
QDDSKVF

SEQ ID NO: 61 - protein, 7 aa, Artificial Sequence
  Synthetic construct
  MOD_RES (1)..(1): decanoyl
QDDSKVF

SEQ ID NO: 62 - protein, 7 aa, Artificial Sequence
  Synthetic construct
  MOD_RES (1)..(1): lauroyl
QDDSKVF

SEQ ID NO: 63 - protein, 7 aa, Artificial Sequence
  Synthetic construct
  MOD_RES (1)..(1): myristoyl
QDDSKVF

SEQ ID NO: 64 - protein, 7 aa, Artificial Sequence
  Synthetic construct
  MOD_RES (1)..(1): palmitoyl
QDDSKVF

SEQ ID NO: 65 - protein, 7 aa, Artificial Sequence
  Synthetic construct
  MOD_RES (1)..(1): stearoyl
QDDSKVF

SEQ ID NO: 66 - protein, 7 aa, Artificial Sequence
  Synthetic construct
  MOD_RES (1)..(1): palmitoleoyl
QDDSKVF

SEQ ID NO: 67 - protein, 7 aa, Artificial Sequence
  Synthetic construct
  MOD_RES (1)..(1): arachidoyl
QDDSKVF

SEQ ID NO: 68 - protein, 7 aa, Artificial Sequence
  Synthetic construct
  MOD_RES (1)..(1): behenoyl
QDDSKVF

SEQ ID NO: 69 - protein, 7 aa, Artificial Sequence
  Synthetic construct
  MOD_RES (1)..(1): oleoyl
QDDSKVF

SEQ ID NO: 70 - protein, 7 aa, Artificial Sequence
  Synthetic construct
  MOD_RES (1)..(1): ricinoleoyl
QDDSKVF

SEQ ID NO: 71 - protein, 7 aa, Artificial Sequence
  Synthetic construct
  MOD_RES (1)..(1): linolenoyl
QDDSKVF

SEQ ID NO: 72 - protein, 7 aa, Artificial Sequence
  Synthetic construct
  MOD_RES (1)..(1): vacceoyl
QDDSKVF

SEQ ID NO: 73 - protein, 7 aa, Artificial Sequence
  Synthetic construct
  MOD_RES (1)..(1): gadoleoyl
QDDSKVF

SEQ ID NO: 74 - protein, 7 aa, Artificial Sequence
  Synthetic construct
  MOD_RES (1)..(1): erucoyl
QDDSKVF

SEQ ID NO: 75 - protein, 7 aa, Artificial Sequence
  Synthetic construct
  MOD_RES (1)..(1): cetoleoyl
QDDSKVF

SEQ ID NO: 76 - protein, 7 aa, Artificial Sequence
  Synthetic construct
  MOD_RES (1)..(1): nervonoyl
QDDSKVF

SEQ ID NO: 77 - protein, 7 aa, Artificial Sequence
  Synthetic construct
  MOD_RES (1)..(1): adrenoyl
QDDSKVF

SEQ ID NO: 78 - protein, 7 aa, Artificial Sequence
  Synthetic construct
  MOD_RES (1)..(1): alpha-linolenoyl
QDDSKVF

SEQ ID NO: 79 - protein, 7 aa, Artificial Sequence
  gamma-linolenoyl
QDDSKVF

SEQ ID NO: 80 - protein, 7 aa, Artificial Sequence
  Synthetic construct
  MOD_RES (1)..(1): EPA
QDDSKVF

SEQ ID NO: 81 - protein, 7 aa, Artificial Sequence
  Synthetic construct
  MOD_RES (1)..(1): DHA
QDDSKVF

SEQ ID NO: 82 - protein, 7 aa, Artificial Sequence
  Synthetic construct
  MOD_RES (1)..(1): butanoyl
VFKEGSC

SEQ ID NO: 83 - protein, 7 aa, Artificial Sequence
  Synthetic construct
  MOD_RES (1)..(1): hexanoyl
VFKEGSC

SEQ ID NO: 84 - protein, 7 aa, Artificial Sequence
  Synthetic construct
VFKEGSC

SEQ ID NO: 85 - protein, 7 aa, Artificial Sequence
  Synthetic construct
  MOD_RES (1)..(1): decanoyl
VFKEGSC

SEQ ID NO: 86 - protein, 7 aa, Artificial Sequence
  Synthetic construct
  MOD_RES (1)..(1): lauroyl
VFKEGSC

SEQ ID NO: 87 - protein, 7 aa, Artificial Sequence
  Synthetic construct
  MOD_RES (1)..(1): myristoyl
VFKEGSC

SEQ ID NO: 88 - protein, 7 aa, Artificial Sequence
  Synthetic construct
  MOD_RES (1)..(1): palmitoyl
VFKEGSC

SEQ ID NO: 89 - protein, 7 aa, Artificial Sequence
  Synthetic construct
  MOD_RES (1)..(1): stearoyl
VFKEGSC

SEQ ID NO: 90 - protein, 7 aa, Artificial Sequence
  Synthetic construct
  MOD_RES (1)..(1): palmitoleoyl
VFKEGSC

SEQ ID NO: 91 - protein, 7 aa, Artificial Sequence
  Synthetic construct
  MOD_RES (1)..(1): arachidoyl
VFKEGSC

SEQ ID NO: 92 - protein, 7 aa, Artificial Sequence
  Synthetic construct
  MOD_RES (1)..(1): behenoyl
VFKEGSC

SEQ ID NO: 93 - protein, 7 aa, Artificial Sequence
  Synthetic construct
  MOD_RES (1)..(1): oleoyl
VFKEGSC

SEQ ID NO: 94 - protein, 7 aa, Artificial Sequence
  Synthetic construct
  MOD_RES (1)..(1): ricinoleoyl
VFKEGSC

SEQ ID NO: 95 - protein, 7 aa, Artificial Sequence
  Synthetic construct
  MOD_RES (1)..(1): linolenoyl
VFKEGSC

SEQ ID NO: 96 - protein, 7 aa, Artificial Sequence
  Synthetic construct
  MOD_RES (1)..(1): vacceoyl
VFKEGSC

SEQ ID NO: 97 - protein, 7 aa, Artificial Sequence
  Synthetic construct
  MOD_RES (1)..(1): gadoleoyl
VFKEGSC

SEQ ID NO: 98 - protein, 7 aa, Artificial Sequence
  Synthetic construct
  MOD_RES (1)..(1): erucoyl
VFKEGSC

SEQ ID NO: 99 - protein, 7 aa, Artificial Sequence
  Synthetic construct
  MOD_RES (1)..(1): cetoleoyl
VFKEGSC

SEQ ID NO: 100 - protein, 7 aa, Artificial Sequence
  Synthetic construct
  MOD_RES (1)..(1): nervonoyl
VFKEGSC

SEQ ID NO: 101 - protein, 7 aa, Artificial Sequence
  Synthetic construct
  MOD_RES (1)..(1): adrenoyl
VFKEGSC

SEQ ID NO: 102 - protein, 7 aa, Artificial Sequence
  Synthetic construct
  MOD_RES (1)..(1): alpha-linolenoyl
VFKEGSC

SEQ ID NO: 103 - protein, 7 aa, Artificial Sequence
  Synthetic construct
  MOD_RES (1)..(1): gamma-linolenoyl
VFKEGSC

SEQ ID NO: 104 - protein, 7 aa, Artificial Sequence
  Synthetic construct
  MOD_RES (1)..(1): EPA
VFKEGSC

SEQ ID NO: 105 - protein, 7 aa, Artificial Sequence
  Synthetic construct
  MOD_RES (1)..(1): DHA
VFKEGSC